(19) United States
(12) Patent Application Publication
(10) Pub. No.: US 2025/0122285 A1
(43) Pub. Date: Apr. 17, 2025

(54) INTERLEUKIN-2 MUTEINS, FUSION PROTEINS, PHARMACEUTICAL COMPOSITIONS, AND THERAPEUTIC APPLICATIONS

(71) Applicant: ANWITA BIOSCIENCES, INC., San Carlos, CA (US)

(72) Inventors: Ziyang Zhong, Belmont, CA (US); Fan Ye, Mountain View, CA (US); Jianing Huang, San Mateo, CA (US)

(21) Appl. No.: 18/291,286

(22) PCT Filed: Jul. 27, 2022
(86) PCT No.: PCT/US2022/074181

§ 371 Date: Jan. 23, 2024

Related U.S. Application Data
(60) Provisional application No. 63/364,718, filed on May 13, 2022.
(60) Provisional application No. 63/226,178, filed on Jul. 28, 2021.

(51) Int. Cl. C07K 16/28; A61K 38/20; A61K 39/00; A61K 39/395; A61P 35/00; C07K 14/55

(52) U.S. Cl. CPC C07K 16/2818; A61K 38/2013; A61K 39/39558; A61P 35/00; C07K 14/55; A61K 2039/505; C07K 2319/33

(57) ABSTRACT

Provided herein are interleukin-2 muteins and fusion proteins comprising an interleukin-2 mutein and a half-life-extension domain. Also provided herein are their pharmaceutical compositions and methods of use for treating, preventing, or ameliorating one or more symptoms of a proliferative disease.

### CROSS REFERENCE TO RELATED APPLICATION

This application claims the benefit of the priority of U.S. Provisional Application Nos. 63/226,178, filed Jul. 28, 2021; and 63/364,718, filed May 13, 2021; the disclosure of each of which is incorporated herein by reference in its entirety.

### FIELD

Provided herein are interleukin-2 muteins and fusion proteins comprising an interleukin-2 mutein and a half-life-extension domain. Also provided herein are their pharmaceutical compositions and methods of use for treating, preventing, or ameliorating one or more symptoms of a proliferative disease.

### REFERENCE TO A SEQUENCE LISTING

The present specification is being filed with a Sequence Listing entitled 216A015WO01_SEQLIST_ST26.XML of 310,277 bytes in size and created Jul. 26, 2022; the content of which is incorporated herein by reference in its entirety.

### BACKGROUND

Dysregulation of the host immune system is one important immune resistance mechanism for cancer. Hanahan and Weinberg, Cell 2011, 144, 646-74; Pardoll, Nat. Rev. Cancer 2012, 12, 252-64. One class of immunotherapy is agents targeting specific checkpoint proteins that play critical roles in regulating T-cell activation and proliferation. Waldman et al., Nat. Rev. Immunol. 2020, 20, 651-68. These proteins function as co-receptors on the surfaces of T-cells to help regulate T-cell responses following T-cell activation. Wolchok et al., Cancer J. 2010, 16, 311-7. The two best characterized checkpoint proteins are cytotoxic T-lymphocyte antigen 4 (CTLA-4) and programmed cell death-1 (PD-1), both serve as negative regulators of T-cell activation. Waldman et al., Nat. Rev. Immunol. 2020, 20, 651-68. T-cell activation induces expression of CTLA-4 and PD-1, thereby inhibits further T-cell activation and proliferation. Pardoll, Nat. Rev. Cancer 2012, 12, 252-64. Immune checkpoint blockade removes such inhibitory signals and unleashes antitumor immune responses. Id.; Sharma and Allison, Science 2015, 348, 56-61. Ipilimumab, a CTLA-4 blocking antibody, was the first immune checkpoint inhibitor approved by the FDA for cancer treatment. Id. Several anti-PD-1 antibodies have since been approved for cancer treatment. Gong et al., J. Immunother. Cancer 2018, 6, 8. While immunotherapy has been a major advance in cancer treatment, up to 85 percent of patients whose cancer is treated with checkpoint inhibitors do not benefit. Haslam and Prasad, JAMA Netw. Open 2019, 2, e192535.

An interleukin-2 (IL-2) is a pleiotropic cytokine that orchestrates the proliferation, survival, and function of both immune effector (Teff) cells and regulatory T (Treg) cells to maintain immune homeostasis. Bluestone, N. Engl. J. Med. 2011, 365, 2129-31; Boyman et al., Nat. Rev. Immunol. 2012, 12, 180-90. The IL-2 drives T-cell growth, augments natural killer (NK) cytolytic activity, induces the differentiation of regulatory T (Treg) cells, and mediates activation-induced cell death. Liao et al., Curr. Opin. Immunol. 2011, 23, 598-604.

An interleukin-2 receptor (IL-2R) exists in three different forms generated from three different interleukin-2 receptor chains: a chain (IL-2Rα or CD25), β chain (IL-2Rβ or CD122), and γ chain (IL-2Rγ, γ_{c}, or CD132). Wang et al., Science 2005, 310, 1159-63. The IL-2 binds the IL-2Rα with a low affinity (K_{d}≈10 nM). Id. From a crystal structure of a quaternary IL-2 signaling complex, fifteen amino acid residues (K35, T37, R38, T41, F42, K43, F44, Y45, E61, E62, K64, P65, E68, L72, and Y107) on the IL-2 are identified as interface residues between the IL-2 and IL-2Rα. Stauber et al., Proc. Natl. Acad. Sci. U.S.A. 2006, 103, 2788-93. The IL-2 binds a heterodimeric complex of the IL-2Rβ and IL-2Rγ, expressed on memory T cells and NK cells, with an intermediate affinity (K_{d}≈1 nM). Wang et al., Science 2005, 310, 1159-63. The IL-2 binds a heterotrimeric complex of the IL-2Rα, IL-2Rβ, and IL-2Rγ, expressed on Treg cells, with a high affinity (K_{d}≈10 pM). Id. The IL-2 binds the IL-2Rβ alone with a dissociation constant (K_{d}) of about 100 nM and has no detectable finding affinity for the IL-2Rγ alone. Id. The IL-2Rα by itself has no signal-transducing activity. Id. The IL-2 signals through the intermediate-affinity heterodimeric IL-2Rβ/γ complex or the high-affinity heterotrimeric IL-2Rα/β/γ complex. Liao et al., Curr. Opin. Immunol. 2011, 23, 598-604. The binding of the IL-2 to the intermediate-affinity heterodimeric IL-2Rβ/γ complex leads to the activation and proliferation of immunostimulatory Teff cells, while the binding of the IL-2 to the high-affinity heterotrimeric IL-2Rα/β/γ complex results in the activation and proliferation of immunosuppressive Treg cells. Malek et al., Immunity 2010, 33, 153-65; Bluestone, N. Engl. J. Med. 2011, 365, 2129-31; Boyman et al., Nat. Rev. Immunol. 2012, 12, 180-90; Spangler et al., Annu. Rev. Immunol. 2015, 33, 139-67. This dual opposing functions of immunostimulation and immunosuppression pose a major challenge in developing the IL-2 as a safe and effective therapeutic agent. Skrombolas et al., Expert Rev. Clin. Immunol. 2014, 10, 207-17; Abbas et al., Sci. Immunol. 2018, 3, eaat1482.

Aldesleukin, a recombinant human IL-2, was approved by the FDA for metastatic renal cell carcinoma in 1992 and for metastatic melanoma in 1998. Rosenberg, J. Immunol. 2014, 192, 5451-8. Patients with metastatic melanoma or renal cancer experience a 5 to 10% rate of complete cancer regression, with an additional 10% experiencing a partial regression. Atkins et al., J. Clin. Oncol. 1999, 17, 2105-16; Klapper et al., Cancer 2008, 113, 293-301. Approximately 70% of complete responders to the IL-2 therapy do not recur. Rosenberg, Sci. Transl. Med. 2012, 4, 127ps8. However, the success of the IL-2 as an immunotherapy for cancer has been hampered by its severe toxicities and limited efficacy. One major limiting factor for its efficacy as an anticancer agent is immunosuppression resulting from the IL-2-driven preferential expansion of Treg cells. Abbas et al., Sci. Immunol. 2018, 3, eaat1482. Moreover, for the IL-2 to be effective in cancer treatment, a high dose therapeutic schedule is required. Bluestone, N. Engl. J. Med. 2011, 365, 2129-31; Abbas et al., Sci. Immunol. 2018, 3, eaat1482. This dosing regimen, however, causes vascular leak syndrome and results in the limited application of IL-2 in cancer treatment. Abbas et al., Sci. Immunol. 2018, 3, eaat1482.

Despite the advances in cancer treatment, cancer remains a major worldwide public health problem. It was estimated that there will be 1,898,160 new cancer cases diagnosed and 608,570 cancer deaths in the US alone in 2021. Cancer Facts & Figures. 2021. Therefore, there is a need for an effective therapy for cancer treatment.

### SUMMARY OF THE DISCLOSURE

Provided herein is an interleukin-2 (IL-2) mutein having attenuated binding affinities to an IL-2Rα and a heterodimeric IL-2Rβ/γ complex as compared to a wild-type IL-2.

Also provided herein is an IL-2 mutein comprising (i) an amino acid substitution at position L18, Q126, or S130; and (ii) a replacement of the amino acid residues from positions N29 to L40 with a peptide comprising an amino acid sequence of an IL-15 hinge or a fragment thereof; a disulfide bond formed between two amino acid residues from positions N30 to L80; or an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, S87, N88, V91, I92, E95, Y107, D109, T111, or S127.

Additionally provided herein is an IL-2 mutein comprising (i) an amino acid substitution at position L18, Q126, or S130; (ii) a replacement of the amino acid residues from positions N29 to L40 with a peptide comprising an amino acid sequence of an IL-15 hinge or a fragment thereof; and (iii) optionally an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, S87, N88, V91, I92, E95, Y107, D109, T111, or S127.

Furthermore, provided herein is a fusion protein comprising an IL-2 domain and a half-life-extension domain.

Provided herein is a fusion protein comprising an IL-2 domain and an albumin binding domain as a half-life-extension domain.

Provided herein is a fusion protein comprising an IL-2 domain, an albumin binding domain, and optionally a peptide linker; wherein a carboxyl terminus (C-terminus) of the albumin binding domain is connected to the amino terminus (N-terminus) of the IL-2 domain directly or via the peptide linker.

Provided herein is a fusion protein comprising an IL-2 domain, a PD-1 binding domain, and an albumin binding domain.

Provided herein is a fusion protein comprising an IL-2 domain, a PD-1 binding domain, an albumin binding domain, and optionally first and second peptide linkers; wherein a C-terminus of the PD-1 binding domain is connected to an N-terminus of the albumin binding domain directly or via the first peptide linker, and a C-terminus of the albumin binding domain is connected to the N-terminus of the IL-2 domain directly or via the second peptide linker.

Provided herein is a fusion protein comprising an IL-2 domain, first and second PD-1 binding domains, an albumin binding domain, and optionally first, second, and third peptide linkers; wherein a C-terminus of the first PD-1 binding domain is connected to an N-terminus of the second PD-1 binding domain directly or via the first peptide linker, a C-terminus of the second PD-1 binding domain is connected to an N-terminus of the albumin binding domain directly or via the second peptide linker, and a C-terminus of the albumin binding domain is connected to the N-terminus of the IL-2 domain directly or via the third peptide linker.

Provided herein is a fusion protein comprising an IL-2 domain and a fragment crystallizable (Fc) domain as a half-life-extension domain.

Provided herein is a fusion protein comprising an IL-2 domain, an Fc domain comprising first and second peptide chains, and optionally a peptide linker; wherein the C-terminus of the first peptide chain of the Fc domain is connected to the N-terminus of the IL-2 domain directly or via the peptide linker.

Provided herein is a fusion protein comprising first and second IL-2 domains, an Fc domain comprising first and second peptide chains, and optionally first and second peptide linkers; wherein the C-terminus of the first peptide chain of the Fc domain is connected to the N-terminus of the first IL-2 domain directly or via the first peptide linker, and the C-terminus of the second peptide chain of the Fc domain is connected to the N-terminus of the second IL-2 domain directly or via the second peptide linker.

Provided herein is a fusion protein comprising an IL-2 domain, a PD-1 binding domain, and an Fc domain.

Provided herein is a fusion protein comprising an IL-2 domain, first and second PD-1 binding domains, an Fc domain comprising first and second peptide chains, and optionally a peptide linker; wherein a C-terminus of the first PD-1 binding domain is connected to the N-terminus of the first peptide chain of the Fc domain directly or via a peptide linker, and the C-terminus of the first peptide chain of the Fc domain is connected to the N-terminus of the IL-2 domain directly or via the peptide linker; and wherein a C-terminus of the second PD-1 binding domain is connected to the N-terminus of the second peptide chain of the Fc domain directly or via a peptide linker.

Provided herein is a fusion protein comprising first and second IL-2 domains, first and second PD-1 binding domains, an Fc domain comprising first and second peptide chains, and optionally first and second peptide linkers; wherein a C-terminus of the first PD-1 binding domain is connected to the N-terminus of the first peptide chain of the Fc domain directly or via a peptide linker, and the C-terminus of the first peptide chain of the Fc domain is connected to the N-terminus of the IL-2 domain directly or via the peptide linker; and wherein a C-terminus of the second PD-1 binding domain is connected to the N-terminus of the second peptide chain of the Fc domain directly or via a peptide linker, and the C-terminus of the second peptide chain of the Fc domain is connected to the N-terminus of the second IL-2 domain directly or via the second peptide linker.

Provided herein is a fusion protein comprising an IL-2 domain and an anti-PD-1 antibody.

Provided herein is a fusion protein comprising an IL-2 domain, an anti-PD-1 antibody comprising first and second light chains and first and second heavy chains, and optionally a peptide linker, wherein the C-terminus of the first heavy chain of the anti-PD-1 antibody is connected to the N-terminus of the IL-2 domain directly or via the peptide linker.

Provided herein is a fusion protein comprising first and second IL-2 domains, an anti-PD-1 antibody comprising first and second light chains and first and second heavy chains, and optionally first and second peptide linkers; wherein the C-terminus of the first heavy chain of the anti-PD-1 antibody is connected to the N-terminus of the first IL-2 domain directly or via the first peptide linker; and wherein the C-terminus of the second heavy chain of the anti-PD-1 antibody is connected to the N-terminus of the second IL-2 domain directly or via the second peptide linker.

Provided herein is a pharmaceutical composition comprising an IL-2 mutein or fusion protein, and a pharmaceutically acceptable excipient.

Provided herein is a method of treating, preventing, or ameliorating one or more symptoms of a disorder, disease, or condition mediated by a PD-1 in a subject, comprising administering to the subject in need thereof a therapeutically effective amount of an IL-2 mutein or fusion protein.

Provided herein is a method of treating, preventing, or ameliorating one or more symptoms of a disorder, disease, or condition mediated by an IL-2 in a subject, comprising administering to the subject in need thereof a therapeutically effective amount of an IL-2 mutein or fusion protein.

Provided herein is a method of treating, preventing, or ameliorating one or more symptoms of a proliferative disease in a subject, comprising administering to the subject in need thereof a therapeutically effective amount of an IL-2 mutein or fusion protein.

Provided herein is a method of inhibiting the growth of a cell, comprising contacting the cell with an effective amount of an IL-2 mutein or fusion protein.

### BRIEF DESCRIPTION OF THE DRAWINGS

FIG. 1 shows the configurations of exemplary fusion proteins: (i) an IL-2 fusion protein comprising first and second IL-2 domains, and a PD-L1 binding domain, e.g., an intact anti-PD-1 antibody comprising first and second light chains and first and second heavy chains; wherein the C-terminus of the first heavy chain of the anti-PD-1 antibody is connected to the N-terminus of the first IL-2 domain via a peptide linker, and the C-terminus of the second heavy chain of the anti-PD-1 antibody is connected to the N-terminus of the second IL-2 domain via a peptide linker; and (ii) an IL-2 fusion protein comprising an IL-2 domain, and a PD-L1 binding domain, e.g., an intact anti-PD-1 antibody comprising first and second light chains and first and second heavy chains; wherein the C-terminus of the first heavy chain of the anti-PD-1 antibody is connected to the N-terminus of the IL-2 domain via a peptide linker; and wherein the anti-PD-1 antibody is in a knobs-into-holes configuration.

FIG. 2 shows the configurations of exemplary fusion proteins: (i) an IL-2 fusion protein comprising first and second IL-2 domains, first and second PD-1 binding domains, e.g., anti-PD-1 V_{H}H single domain antibodies, and a Fc domain comprising first and second peptide chains; wherein the C-terminus of the first anti-PD-1 V_{H}H single domain antibody is connected to the N-terminus of the first peptide chain of the Fc domain directly or via a peptide linker, and the C-terminus of the first peptide chain of the Fc domain is connected to the N-terminus of the first IL-2 domain via a peptide linker; and wherein the C-terminus of the second anti-PD-1 V_{H}H single domain antibody is connected to the N-terminus of the second peptide chain of the Fc domain directly or via a peptide linker, and the C-terminus of the second peptide chain of the Fc domain is connected to the N-terminus of the second IL-2 domain via a peptide linker; and (ii) an IL-2 fusion protein comprising an IL-2 domain, first and second PD-1 binding domains, e.g., anti-PD-1 V_{H}H single domain antibodies, and a Fc domain comprising first and second peptide chains; wherein the C-terminus of the first anti-PD-1 V_{H}H single domain antibody is connected to the N-terminus of the first peptide chain of the Fc domain directly or via a peptide linker, and the C-terminus of the first peptide chain of the Fc domain is connected to the N-terminus of the first IL-2 domain via a peptide linker; wherein the C-terminus of the second anti-PD-1 V_{H}H single domain antibody is connected to the N-terminus of the second peptide chain of the Fc domain directly or via a peptide linker; and wherein the Fc domain is in a knobs-into-holes configuration.

FIG. 3 shows the configurations of exemplary fusion proteins: (i) an IL-2 fusion protein comprising an IL-2 domain, a PD-L1 binding domain, e.g., an anti-PD-1 V_{H}H single domain antibody (sdAb), and an albumin binding domain, e.g., an anti-HSA V_{H}H sdAb; wherein the C-terminus of the anti-PD-1 V_{H}H sdAb is connected to the N-terminus of the anti-HSA V_{H}H sdAb via a peptide linker, and the C-terminus of the anti-HSA V_{H}H sdAb via a peptide linker is connected to the N-terminus of the IL-2 domain via a peptide linker; (ii) an IL-2 fusion protein comprising an IL-2 domain, first and second PD-L1 binding domains, e.g., anti-PD-1 V_{H}H sdAbs, and an albumin binding domain, e.g., an anti-HSA V_{H}H sdAb; wherein the C-terminus of the first anti-PD-1 V_{H}H sdAb is connected to the N-terminus of the second anti-PD-1 V_{H}H sdAb via a peptide linker, the C-terminus of the second anti-PD-1 V_{H}H sdAb is connected to the N-terminus of the anti-HSA V_{H}H sdAb via a peptide linker, and the C-terminus of the anti-HSA V_{H}H sdAb via a peptide linker is connected to the N-terminus of the IL-2 domain via a peptide linker; and (iii) an IL-2 fusion protein comprising an IL-2 domain, a PD-L1 binding domain, e.g., an anti-PD-1 single-chain fragment variable (scFv), and an albumin binding domain, e.g., an anti-HSA V_{H}H sdAb; wherein the C-terminus of the anti-PD-1 scFv is connected to the N-terminus of the anti-HSA V_{H}H sdAb via a peptide linker, and the C-terminus of the anti-HSA V_{H}H sdAb is connected to the N-terminus of the IL-2 domain via a peptide linker.

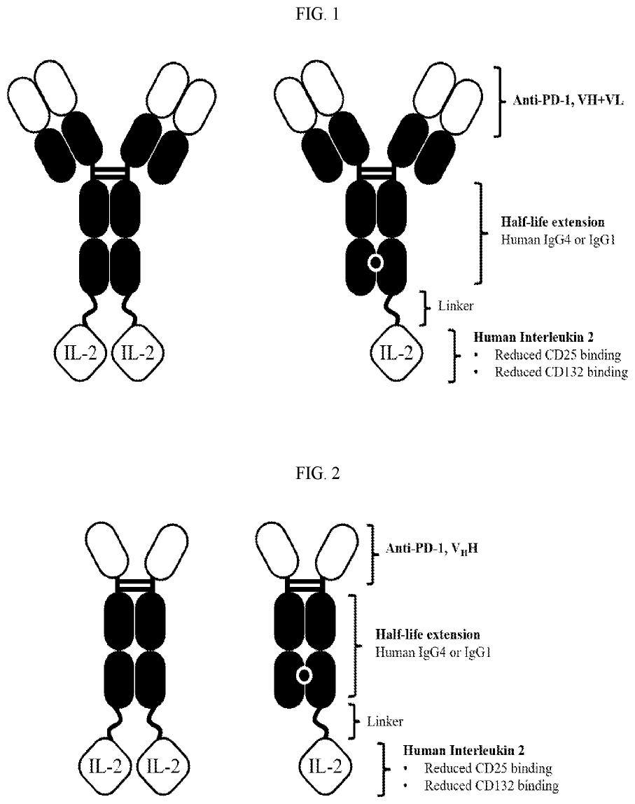

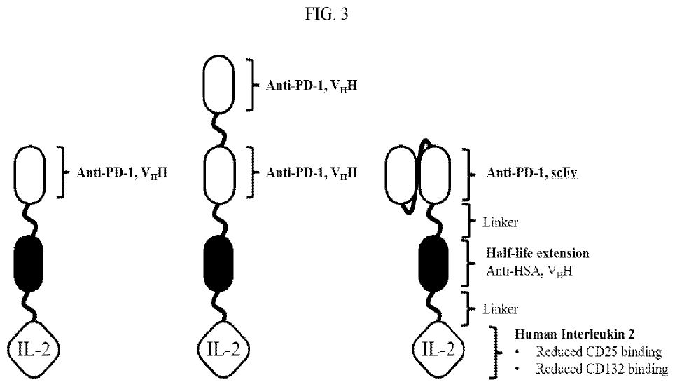

### DETAILED DESCRIPTION

To facilitate understanding of the disclosure set forth herein, a number of terms are defined below.

Generally, the nomenclature used herein and the laboratory procedures in biochemistry, biology, cell biology, immunology, molecular biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The term “therapeutically effective amount” or “effective amount” is meant to include the amount of a compound (e.g., a polypeptide or fusion protein) that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound (e.g., a polypeptide or fusion protein) that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “substantially pure” and “substantially homogeneous,” when referring to a compound, mean sufficiently homogeneous to appear free of readily detectable impurities as determined by standard analytical methods used by one of ordinary skill in the art, including, but not limited to, gel electrophoresis, high performance liquid chromatography (HPLC), and mass spectrometry (MS); or sufficiently pure such that further purification would not detectably alter the physical, chemical, biological, and/or pharmacological properties, such as enzymatic and biological activities, of the compound. In certain embodiments, “substantially pure” or “substantially homogeneous” refers to a collection of molecules, wherein at least about 50%, at least about 70%, at least about 80%, at least about 90%, at least about 95%, at least about 98%, at least about 99%, or at least about 99.5% by weight of the molecules are a single compound as determined by a standard analytical method.

### Interleukin-2 Muteins

In one embodiment, provided herein is an IL-2 mutein comprising, as set forth in SEQ ID NO: 179, 180, 181, 182, 183, or 184, (i) an amino acid substitution at position L18, Q126, or S130; and (ii) a replacement of the amino acid residues from positions N29 to L40 with a peptide comprising an amino acid sequence of an IL-15 hinge or a fragment thereof (“an IL-15 hinge fragment-containing peptide”); a disulfide bond formed between two amino acid residues from positions N30 to L80; or an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, S87, N88, V91, I92, E95, Y107, D109, T111, or S127.

In one embodiment, the IL-2 mutein provided herein comprises: (i) an amino acid substitution at position L18, Q126, or S130; and (ii) a replacement of the amino acid residues from positions N29 to L40 with an IL-15 hinge fragment-containing peptide; a disulfide bond formed between two amino acid residues from positions N30 to L80; an amino acid substitution at position E15, H16, D20, K32, K76, S87, N88, or 192; and/or an amino acid substitution at a position from R38 to Y45.

In another embodiment, the IL-2 mutein provided herein comprises: (i) an amino acid substitution at position L18, Q126, or S130; and (ii) a replacement of the amino acid residues from positions N29 to L40 with an IL-15 hinge fragment-containing peptide; a disulfide bond formed between two amino acid residues from positions N30 to L80; or an amino acid substitution at position E15, H16, D20, K32, R38, L40, F42, K76, S87, N88, or 192.

In yet another embodiment, the IL-2 mutein provided herein comprises: (i) an amino acid substitution at position L18 or Q126; and (ii) a replacement of the amino acid residues from positions N29 to L40 with an IL-15 hinge fragment-containing peptide; or an amino acid substitution at position 192.

In yet another embodiment, the IL-2 mutein provided herein comprises: (i) an amino acid substitutions at positions L18 and Q126; and (ii) a replacement of the amino acid residues from positions N29 to L40 with an IL-15 hinge fragment-containing peptide.

In still another embodiment, the IL-2 mutein provided herein comprises: (i) an amino acid substitution at position Q126; and (ii) a replacement of the amino acid residues from positions N29 to L40 with an IL-15 hinge fragment-containing peptide; and an amino acid substitution at position 192.

In certain embodiments, the amino acid residue at position E15 is one of the twenty natural amino acids (i.e., Ala (A), Cys (C), Asp (D), Glu (E), Phe (F), Gly (G), His (H), Ile (I), Lys (K), Leu (L), Met (M), Asn (N), Pro (P), Gln (Q), Arg (R), Ser (S), Thr (T), Val (V), Trp (W), and Tyr (Y)) other than E. In certain embodiments, the amino acid residue at position E15 is K.

In certain embodiments, the amino acid residue at position H16 is one of the twenty natural amino acids other than H. In certain embodiments, the amino acid residue at position H16 is E, F, I, or V. In certain embodiments, the amino acid residue at position H16 is E, I, or V. In certain embodiments, the amino acid residue at position H16 is E. In certain embodiments, the amino acid residue at position H16 is F. In certain embodiments, the amino acid residue at position H16 is I. In certain embodiments, the amino acid residue at position H16 is V.

In certain embodiments, the amino acid residue at position L18 is one of the twenty natural amino acids other than L. In certain embodiments, the amino acid residue at position L18 is C or S. In certain embodiments, the amino acid residue at position L18 is C. In certain embodiments, the amino acid residue at position L18 is C and the amino acid residue at position C125 is C. In certain embodiments, the amino acid residue at position L18 is S.

In certain embodiments, the amino acid residue at position D20 is one of the twenty natural amino acids other than D. In certain embodiments, the amino acid residue at position D20 is A, E, K, or T. In certain embodiments, the amino acid residue at position D20 is A. In certain embodiments, the amino acid residue at position D20 is E. In certain embodiments, the amino acid residue at position D20 is K. In certain embodiments, the amino acid residue at position D20 is T.

In certain embodiments, the amino acid residue at position K32 is one of the twenty natural amino acids other than K. In certain embodiments, the amino acid residue at position K32 is D, E, or Q. In certain embodiments, the amino acid residue at position K32 is D. In certain embodiments, the amino acid residue at position K32 is E. In certain embodiments, the amino acid residue at position K32 is Q.

In certain embodiments, the amino acid residue at position R38 is one of the twenty natural amino acids other than R. In certain embodiments, the amino acid residue at position R38 is E or N. In certain embodiments, the amino acid residue at position R38 is E. In certain embodiments, the amino acid residue at position R38 is N.

In certain embodiments, the amino acid residue at position L40 is one of the twenty natural amino acids other than L. In certain embodiments, the amino acid residue at position L40 is S or T. In certain embodiments, the amino acid residue at position L40 is S. In certain embodiments, the amino acid residue at position L40 is T.

In certain embodiments, the amino acid residue at position F42 is one of the twenty natural amino acids other than F. In certain embodiments, the amino acid residue at position F42 is A, C, K, or N. In certain embodiments, the amino acid residue at position F42 is A or K. In certain embodiments, the amino acid residue at position F42 is A. In certain embodiments, the amino acid residue at position F42 is C. In certain embodiments, the amino acid residue at position F42 is K. In certain embodiments, the amino acid residue at position F42 is N.

In certain embodiments, the amino acid residue at position K76 is one of the twenty natural amino acids other than K. In certain embodiments, the amino acid residue at position K76 is D, E, or Q. In certain embodiments, the amino acid residue at position K76 is D. In certain embodiments, the amino acid residue at position K76 is E. In certain embodiments, the amino acid residue at position K76 is Q.

In certain embodiments, the amino acid residue at position S87 is one of the twenty natural amino acids other than S. In certain embodiments, the amino acid residue at position S87 is D or E. In certain embodiments, the amino acid residue at position S87 is D. In certain embodiments, the amino acid residue at position S87 is E.

In certain embodiments, the amino acid residue at position N88 is one of the twenty natural amino acids other than N. In certain embodiments, the amino acid residue at position N88 is A.

In certain embodiments, the amino acid residue at position 192 is one of the twenty natural amino acids other than I. In certain embodiments, the amino acid residue at position 192 is A, D, E, or G. In certain embodiments, the amino acid residue at position 192 is A. In certain embodiments, the amino acid residue at position 192 is D. In certain embodiments, the amino acid residue at position 192 is E. In certain embodiments, the amino acid residue at position 192 is G.

In certain embodiments, the amino acid residue at position Q126 is one of the twenty natural amino acids other than Q. In certain embodiments, the amino acid residue at position Q126 is E, K, R, S, or T. In certain embodiments, the amino acid residue at position Q126 is E, K, or R. In certain embodiments, the amino acid residue at position Q126 is E. In certain embodiments, the amino acid residue at position Q126 is K. In certain embodiments, the amino acid residue at position Q126 is R. In certain embodiments, the amino acid residue at position Q126 is S. In certain embodiments, the amino acid residue at position Q126 is T.

In certain embodiments, the amino acid residue at position S130 is one of the twenty natural amino acids other than S. In certain embodiments, the amino acid residue at position S130 is A, E, Q, or R. In certain embodiments, the amino acid residue at position S130 is A. In certain embodiments, the amino acid residue at position S130 is E. In certain embodiments, the amino acid residue at position S130 is Q. In certain embodiments, the amino acid residue at position S130 is R.

In one embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) a replacement of the amino acid residues from positions N29 to L40 having an amino acid sequence of SEQ ID NO: 217 with an IL-15 hinge fragment-containing peptide; a disulfide bond formed between two different amino acid residues, each independently at position K35, R38, F42, Y45, E62, V69, or L72; or an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In another embodiment, the IL-2 mutein provided herein comprises: (i) an amino acid substitution of L18C, Q126E, Q126K, Q126R, Q126S, Q126T, S130A, S130E, S130Q, or S130R; and (ii) a replacement of the amino acid residues from positions N29 to L40 having an amino acid sequence of SEQ ID NO: 217 with an IL-15 hinge fragment-containing peptide; a disulfide bond formed between two different amino acid residues, each independently at position K35, R38, F42, Y45, E62, V69, or L72; or an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In yet another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) a replacement of the amino acid residues from positions N29 to L40 having an amino acid sequence of SEQ ID NO: 217 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) a replacement of the amino acid residues from positions N29 to L40 having an amino acid sequence of SEQ ID NO: 217 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein provided herein comprises: (i) an amino acid substitution of L18C, L18S, Q126E, Q126K, Q126R, S130A, S130E, S130Q, or S130R; and (ii) a replacement of the amino acid residues from positions N29 to L40 having an amino acid sequence of SEQ ID NO: 217 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein provided herein comprises: (i) an amino acid substitution of L18C, Q126E, Q126K, Q126R, S130A, S130E, S130Q, or S130R; and (ii) a replacement of the amino acid residues from positions N29 to L40 having an amino acid sequence of SEQ ID NO: 217 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein provided herein comprises: (i) an amino acid substitution of L18C, L18S, Q126E, Q126K, Q126R, S130A, S130E, S130Q, or S130R; and (ii) a replacement of the amino acid residues from positions N29 to L40 having an amino acid sequence of SEQ ID NO: 217 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein provided herein comprises: (i) an amino acid substitution of L18C, Q126E, Q126K, or S130R; and (ii) a replacement of the amino acid residues from positions N29 to L40 having an amino acid sequence of SEQ ID NO: 217 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein provided herein comprises: (i) an amino acid substitution of L18S or Q126E; and (ii) a replacement of the amino acid residues from positions N29 to L40 having an amino acid sequence of SEQ ID NO: 217 with an IL-15 hinge fragment-containing peptide; or an amino acid substitution of I92A.

In yet another embodiment, the IL-2 mutein provided herein comprises: (i) an amino acid substitutions of L18S and Q126E; and (ii) a replacement of the amino acid residues from positions N29 to L40 having an amino acid sequence of SEQ ID NO: 217 with an IL-15 hinge fragment-containing peptide.

In still another embodiment, the IL-2 mutein provided herein comprises: (i) an amino acid substitution of Q126E; and (ii) a replacement of the amino acid residues from positions N29 to L40 having an amino acid sequence of SEQ ID NO: 217 with an IL-15 hinge fragment-containing peptide; and an amino acid substitution of I92A.

In one embodiment, the IL-15 hinge fragment-containing peptide comprises an amino acid sequence of SEQ ID NO: 218 or 219. In another embodiment, the IL-15 hinge fragment-containing peptide comprises an amino acid sequence of SEQ ID NO: 218. In yet another embodiment, the IL-15 hinge fragment-containing peptide comprises an amino acid sequence of SEQ ID NO: 219.

In one embodiment, the IL-2 mutein provided herein comprises: (i) an amino acid substitution of L18C, L18S, Q126E, Q126K, or S130R; and (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In another embodiment, the IL-2 mutein provided herein comprises: (i) an amino acid substitution of L18C, Q126E, Q126K, or S130R; and (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein provided herein comprises: (i) an amino acid substitution of L18C, L18S, Q126E, Q126K, or S130R; and (ii) a replacement of the amino acid residues from positions N29 to L40 having an amino acid sequence of SEQ ID NO: 217 with an IL-15 hinge fragment-containing peptide having an amino acid sequence of SEQ ID NO: 218; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein provided herein comprises: (i) an amino acid substitution of L18C, Q126E, Q126K, or S130R; and (ii) a replacement of the amino acid residues from positions N29 to L40 having an amino acid sequence of SEQ ID NO: 217 with an IL-15 hinge fragment-containing peptide having an amino acid sequence of SEQ ID NO: 218; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein provided herein comprises: (i) an amino acid substitution of L18C, L18S, Q126E, Q126K, or S130R; and (ii) a replacement of the amino acid residues from positions N29 to L40 having an amino acid sequence of SEQ ID NO: 217 with an IL-15 hinge fragment-containing peptide having an amino acid sequence of SEQ ID NO: 219; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein provided herein comprises: (i) an amino acid substitution of L18C, Q126E, Q126K, or S130R; and (ii) a replacement of the amino acid residues from positions N29 to L40 having an amino acid sequence of SEQ ID NO: 217 with an IL-15 hinge fragment-containing peptide having an amino acid sequence of SEQ ID NO: 219; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein provided herein comprises: (i) an amino acid substitution of L18S or Q126E; and (ii) a replacement of the amino acid residues from positions N29 to L40 having an amino acid sequence of SEQ ID NO: 217 with an IL-15 hinge fragment-containing peptide having an amino acid sequence of SEQ ID NO: 218; or an amino acid substitution of I92A.

In yet another embodiment, the IL-2 mutein provided herein comprises: (i) an amino acid substitution of L18S or Q126E; and (ii) a replacement of the amino acid residues from positions N29 to L40 having an amino acid sequence of SEQ ID NO: 217 with an IL-15 hinge fragment-containing peptide having an amino acid sequence of SEQ ID NO: 219; or an amino acid substitution of I92A.

In yet another embodiment, the IL-2 mutein provided herein comprises: (i) an amino acid substitutions of L18S and Q126E; and (ii) a replacement of the amino acid residues from positions N29 to L40 having an amino acid sequence of SEQ ID NO: 217 with an IL-15 hinge fragment-containing peptide having an amino acid sequence of SEQ ID NO: 218.

In yet another embodiment, the IL-2 mutein provided herein comprises: (i) an amino acid substitutions of L18S and Q126E; and (ii) a replacement of the amino acid residues from positions N29 to L40 having an amino acid sequence of SEQ ID NO: 217 with an IL-15 hinge fragment-containing peptide having an amino acid sequence of SEQ ID NO: 219.

In yet another embodiment, the IL-2 mutein provided herein comprises: (i) an amino acid substitution of Q126E; and (ii) a replacement of the amino acid residues from positions N29 to L40 having an amino acid sequence of SEQ ID NO: 217 with an IL-15 hinge fragment-containing peptide having an amino acid sequence of SEQ ID NO: 218; and an amino acid substitution of I92A.

In still another embodiment, the IL-2 mutein provided herein comprises: (i) an amino acid substitution of Q126E; and (ii) a replacement of the amino acid residues from positions N29 to L40 having an amino acid sequence of SEQ ID NO: 217 with an IL-15 hinge fragment-containing peptide having an amino acid sequence of SEQ ID NO: 219; and an amino acid substitution of I92A.

In one embodiment, the IL-2 mutein provided herein comprises: (i) an amino acid substitution at position L18, Q126, or S130; and (ii) an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, 587, N88, V91, I92, E95, Y107, D109, T111, or S127.

In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In yet another embodiment, the IL-2 mutein provided herein comprises: (i) an amino acid substitution at position L18, Q126, or S130; and (ii) an amino acid substitution at position E15, H16, D20, K32, R38, L40, F42, K76, S87, N88, or 192.

In yet another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein provided herein comprises: (i) an amino acid substitution of L18C, L18S, Q126E, Q126K, Q126R, S130A, S130E, S130Q, or S130R; and (ii) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein provided herein comprises: (i) an amino acid substitution of L18C, Q126E, Q126K, Q126R, S130A, S130E, S130Q, or S130R; and (ii) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein provided herein comprises: (i) an amino acid substitution of L18C, L18S, Q126E, Q126K, or S130R; and (ii) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In still another embodiment, the IL-2 mutein provided herein comprises: (i) an amino acid substitution of L18C, Q126E, Q126K, or S130R; and (ii) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In one embodiment, the IL-2 mutein provided herein comprises an amino acid substitution at position L18. In another embodiment, the IL-2 mutein provided herein comprises an amino acid substitution of L18C or L18S. In yet another embodiment, the IL-2 mutein provided herein comprises an amino acid substitution of L18C. In still another embodiment, the IL-2 mutein provided herein comprises an amino acid substitution of L18S.

In one embodiment, the IL-2 mutein provided herein comprises an amino acid substitution at position Q126. In another embodiment, the IL-2 mutein provided herein comprises an amino acid substitution of Q126E, Q126K, Q126R, Q126S, or Q126T. In yet another embodiment, the IL-2 mutein provided herein comprises an amino acid substitution of Q126E. In yet another embodiment, the IL-2 mutein provided herein comprises an amino acid substitution of Q126K. In yet another embodiment, the IL-2 mutein provided herein comprises an amino acid substitution of Q126R. In yet another embodiment, the IL-2 mutein provided herein comprises an amino acid substitution of Q126S. In still another embodiment, the IL-2 mutein provided herein comprises an amino acid substitution of Q126T.

In one embodiment, the IL-2 mutein provided herein comprises an amino acid substitution at position S130. In another embodiment, the IL-2 mutein provided herein comprises an amino acid substitution of S130A, S130E, S130Q, or S130R. In yet another embodiment, the IL-2 mutein provided herein comprises an amino acid substitution of S130A. In yet another embodiment, the IL-2 mutein provided herein comprises an amino acid substitution of S130E. In yet another embodiment, the IL-2 mutein provided herein comprises an amino acid substitution of S130Q. In still another embodiment, the IL-2 mutein provided herein comprises an amino acid substitution of S130R.

In one embodiment, the IL-2 mutein provided herein comprises amino acid substitutions at positions L18 and S126. In another embodiment, the IL-2 mutein provided herein comprises an amino acid substitution of L18C or L18S and an amino acid substitution of Q126E, Q126K, Q126R, Q126S, or Q126T. In yet another embodiment, the IL-2 mutein provided herein comprises an amino acid substitution of L18C and an amino acid substitution of Q126E, Q126K, Q126R, Q126S, or Q126T. In yet another embodiment, the IL-2 mutein provided herein comprises amino acid substitutions of L18C and Q126E. In yet another embodiment, the IL-2 mutein provided herein comprises amino acid substitutions of L18C and Q126K. In yet another embodiment, the IL-2 mutein provided herein comprises amino acid substitutions of L18C and Q126R. In yet another embodiment, the IL-2 mutein provided herein comprises amino acid substitutions of L18C and Q126S. In still another embodiment, the IL-2 mutein provided herein comprises amino acid substitutions of L18C and Q126T.

In one embodiment, the IL-2 mutein provided herein comprises an amino acid substitution of L18S and an amino acid substitution of Q126E, Q126K, Q126R, Q126S, or Q126T. In another embodiment, the IL-2 mutein provided herein comprises amino acid substitutions of L18S and Q126E. In yet another embodiment, the IL-2 mutein provided herein comprises amino acid substitutions of L18S and Q126K. In yet another embodiment, the IL-2 mutein provided herein comprises amino acid substitutions of L18S and Q126R. In yet another embodiment, the IL-2 mutein provided herein comprises amino acid substitutions of L18S and Q126S. In still another embodiment, the IL-2 mutein provided herein comprises amino acid substitutions of L18S and Q126T.

In one embodiment, the IL-2 mutein provided herein comprises amino acid substitutions at positions S126 and S130. In another embodiment, the IL-2 mutein provided herein comprises an amino acid substitution of Q126E, Q126K, Q126R, Q126S, or Q126T, and an amino acid substitution of S130A, S130E, S130Q, or S130R. In yet another embodiment, the IL-2 mutein provided herein comprises an amino acid substitution of Q126E, Q126K, Q126R, Q126S, or Q126T, and an amino acid substitution of S130E or S130R.

In one embodiment, the IL-2 mutein provided herein comprises: (i) an amino acid substitution at position L18, Q126, or S130; and (ii) an amino acid substitution at position E15. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of E15K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of E15K.

In one embodiment, the IL-2 mutein provided herein comprises: (i) an amino acid substitution at position L18, Q126, or S130; and (ii) an amino acid substitution at position H16. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of H16E, H16F, H16I, or H16V. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of H16E, H16F, H16I, or H16V. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of H16E, H16I, or H16V. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of H16E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of H16F. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of H16I. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of H16V.

In one embodiment, the IL-2 mutein provided herein comprises: (i) an amino acid substitution at position L18, Q126, or S130; and (ii) an amino acid substitution at position D20. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of D20A, D20E, D20K, or D20T. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of D20A, D20E, D20K, or D20T. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of D20A. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of D20E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of D20K. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of D20T.

In one embodiment, the IL-2 mutein provided herein comprises: (i) an amino acid substitution at position L18, Q126, or S130; and (ii) an amino acid substitution at position K32. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of K32E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of K32E.

In one embodiment, the IL-2 mutein provided herein comprises: (i) an amino acid substitution at position L18, Q126, or S130; and (ii) an amino acid substitution at position R38. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of R38E or R38N. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of R38E or R38N. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of R38E. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of R38N.

In one embodiment, the IL-2 mutein provided herein comprises: (i) an amino acid substitution at position L18, Q126, or S130; and (ii) an amino acid substitution at position L40. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of L40S or L40T. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of L40S or L40T. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of L40S. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of L40T.

In one embodiment, the IL-2 mutein provided herein comprises: (i) an amino acid substitution at position L18, Q126, or S130; and (ii) an amino acid substitution at position F42. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of F42A, F42C, or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of F42A, F42C, or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of F42A or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of F42A. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of F42C. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of F42K.

In one embodiment, the IL-2 mutein provided herein comprises: (i) an amino acid substitution at position L18, Q126, or S130; and (ii) an amino acid substitution at position K76. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of K76E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of K76E.

In one embodiment, the IL-2 mutein provided herein comprises: (i) an amino acid substitution at position L18, Q126, or S130; and (ii) an amino acid substitution at position S87. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of S87D. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of S87D.

In one embodiment, the IL-2 mutein provided herein comprises: (i) an amino acid substitution at position L18, Q126, or S130; and (ii) an amino acid substitution at position N88. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of N88A. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of N88A.

In one embodiment, the IL-2 mutein provided herein comprises: (i) an amino acid substitution at position L18, Q126, or S130; and (ii) an amino acid substitution at position 192. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) an amino acid substitution of I92A, I92D, I92E, or I92G. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of I92A, I92D, I92E, or I92G. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of I92A. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of I92D. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of I92E. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) an amino acid substitution of I92G.

In one embodiment, the IL-2 mutein provided herein comprises amino acid substitutions at positions 192 and S126. In another embodiment, the IL-2 mutein provided herein comprises an amino acid substitution of I92A, I92D, I92E, or I92G, and an amino acid substitution of Q126E, Q126K, Q126R, Q126S, or Q126T. In yet another embodiment, the IL-2 mutein provided herein comprises an amino acid substitution of I92A or I92G, and an amino acid substitution of Q126E, Q126K, Q126R, Q126S, or Q126T. In yet another embodiment, the IL-2 mutein provided herein comprises amino acid substitutions of I92A and Q126E. In yet another embodiment, the IL-2 mutein provided herein comprises amino acid substitutions of I92A and Q126K. In yet another embodiment, the IL-2 mutein provided herein comprises amino acid substitutions of I92A and Q126R. In yet another embodiment, the IL-2 mutein provided herein comprises amino acid substitutions of I92A and Q126S. In still another embodiment, the IL-2 mutein provided herein comprises amino acid substitutions of I92A and Q126T.

In one embodiment, the IL-2 mutein provided herein comprises: (i) an amino acid substitution at position L18, Q126, or S130; and (ii) amino acid substitutions at positions R38 and L40. In another embodiment, the IL-2 mutein provided herein comprises: (i) an amino acid substitution at position L18, Q126, or S130; and (ii) amino acid substitutions at positions E15, R38, and L40. In yet another embodiment, the IL-2 mutein provided herein comprises: (i) an amino acid substitution at position L18, Q126, or S130; and (ii) amino acid substitutions at positions R38, L40, and K76. In still another embodiment, the IL-2 mutein provided herein comprises: (i) an amino acid substitution at position L18, Q126, or S130; and (ii) amino acid substitutions at positions E15, R38, L40, and K76.

In one embodiment, the IL-2 mutein provided herein comprises: (i) an amino acid substitution at position L18, Q126, or S130; and (ii) amino acid substitutions of R38N and L40S or L40T. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions of E15K, R38N and L40S or L40T. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38N and L40S or L40T. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of R38N, L40S or L40T, and K76E. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38N, L40S or L40T, and K76E.

In one embodiment, the IL-2 mutein provided herein comprises: (i) an amino acid substitution at position L18, Q126, or S130; and (ii) amino acid substitutions of R38N and L40S. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions of E15K, R38N and L40S. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38N and L40S. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of R38N, L40S, and K76E. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38N, L40S, and K76E.

In one embodiment, the IL-2 mutein provided herein comprises: (i) an amino acid substitution at position L18, Q126, or S130; and (ii) amino acid substitutions of R38N and L40T. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions of E15K, R38N and L40T. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38N and L40T. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of R38N, L40T, and K76E. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38N, L40T, and K76E.

In one embodiment, the IL-2 mutein provided herein comprises an amino acid sequence of any one of SEQ ID NOs: 185 to 192, wherein the IL-2 mutein comprises an amino acid substitution of L18C, Q126E, Q126K, Q126R, Q126S, Q126T, S130A, S130E, S130Q, or S130R; wherein the amino acid residue at position T3 is T or A, and the amino acid residue at position C125 is C or S; and wherein, when the IL-2 mutein comprises an amino acid substitution of L18C, the amino acid residue at position C125 is C.

In one embodiment, the IL-2 mutein provided herein is glycosylated. In another embodiment, the IL-2 mutein provided herein is N-glycosylated. In yet another embodiment, the IL-2 mutein provided herein is glycosylate at the nitrogen in the side chain of an asparagine residue. In still another embodiment, the IL-2 mutein provided herein comprises: (i) an amino acid substitution at position L18, Q126, or S130; and (ii) an amino acid substitution of R38N that is N-glycosylated.

In one embodiment, the IL-2 mutein provided herein is an N-glycosylated IL-2 mutein comprising: (i) an amino acid substitution at position L18, Q126, or S130; and (ii) amino acid substitutions at positions R38 and L40. In another embodiment, the IL-2 mutein provided herein is an N-glycosylated IL-2 mutein comprising: (i) an amino acid substitution at position L18, Q126, or S130; (ii) amino acid substitutions at positions E15, R38, and L40; and (iii) optionally an amino acid substitution at position H16, D20, K76, S87, N88, or 192. In yet another embodiment, the IL-2 mutein provided herein is an N-glycosylated IL-2 mutein comprising: (i) an amino acid substitution of at position L18, Q126, or S130; (ii) amino acid substitutions at positions R38, L40, and K76; and (iii) optionally an amino acid substitution at position E15, H16, D20, S87, N88, or 192. In still another embodiment, the IL-2 mutein provided herein is an N-glycosylated IL-2 mutein comprising: (i) an amino acid substitution of at position L18, Q126, or S130; (ii) amino acid substitutions at positions E15, R38, L40, and K76; and (iii) optionally an amino acid substitution at position H16, D20, S87, N88, or 192.

In one embodiment, the IL-2 mutein provided herein is an N-glycosylated IL-2 mutein comprising: (i) an amino acid substitution at position L18, Q126, or S130; and (ii) amino acid substitutions of R38N and L40S or L40T. In another embodiment, the IL-2 mutein provided herein is an N-glycosylated IL-2 mutein: (i) an amino acid substitution of L18C, L18S, Q126E, Q126K, Q126R, Q126S, Q126T, S130A, S130E, S130Q, or S130R; and (ii) comprising amino acid substitutions of E15K, R38N and L40S or L40T. In yet another embodiment, the IL-2 mutein provided herein is an N-glycosylated IL-2 mutein: (i) an amino acid substitution of L18C, Q126E, Q126K, Q126R, Q126S, Q126T, S130A, S130E, S130Q, or S130R; and (ii) comprising amino acid substitutions of E15K, R38N and L40S or L40T. In yet another embodiment, the IL-2 mutein provided herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of R38N, L40S or L40T, and K76E. In still another embodiment, the IL-2 mutein provided herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N, L40S or L40T, and K76E.

In one embodiment, the IL-2 mutein provided herein is an N-glycosylated IL-2 mutein comprising: (i) an amino acid substitution at position L18, Q126, or S130; and (ii) amino acid substitutions of R38N and L40S. In another embodiment, the IL-2 mutein provided herein is an N-glycosylated IL-2 mutein comprising: (i) an amino acid substitution of L18C, L18S, Q126E, Q126K, Q126R, Q126S, Q126T, S130A, S130E, S130Q, or S130R; and (ii) amino acid substitutions of E15K, R38N and L40S. In yet another embodiment, the IL-2 mutein provided herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N and L40S. In yet another embodiment, the IL-2 mutein provided herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of R38N, L40S, and K76E. In still another embodiment, the IL-2 mutein provided herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N, L40S, and K76E.

In one embodiment, the IL-2 mutein provided herein is an N-glycosylated IL-2 mutein comprising: (i) an amino acid substitution at position L18, Q126, or S130; and (ii) amino acid substitutions of R38N and L40T. In another embodiment, the IL-2 mutein provided herein is an N-glycosylated IL-2 mutein comprising: (i) an amino acid substitution of L18C, L18S, Q126E, Q126K, Q126R, Q126S, Q126T, S130A, S130E, S130Q, or S130R; and (ii) amino acid substitutions of E15K, R38N and L40T. In yet another embodiment, the IL-2 mutein provided herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N and L40T. In yet another embodiment, the IL-2 mutein provided herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of R38N, L40T, and K76E. In still another embodiment, the IL-2 mutein provided herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N, L40T, and K76E.

In one embodiment, the N-glycosylated IL-2 mutein provided herein comprises an amino acid sequence of any one of SEQ ID NOs: 185 to 192, wherein the N-glycosylated IL-2 mutein comprises an amino acid substitution of L18C, Q126E, Q126K, Q126R, Q126S, Q126T, S130A, S130E, S130Q, or S130R; wherein the amino acid residue at position T3 is T or A, and the amino acid residue at position C125 is C or S; and wherein, when the IL-2 mutein comprises an amino acid substitution of L18C, the amino acid residue at position C125 is C.

In one embodiment, the N-glycosylated IL-2 mutein provided herein comprises one glycan. In another embodiment, the N-glycosylated IL-2 mutein provided herein comprises one glycan attached to the nitrogen in the side chain of an asparagine residue. In yet another embodiment, the N-glycosylated IL-2 mutein provided herein comprises one glycan attached to the nitrogen in the side chain of an asparagine residue at position R38N.

In one embodiment, the N-glycosylated IL-2 mutein provided herein comprises two glycans. In another embodiment, the N-glycosylated IL-2 mutein provided herein comprises two glycans, of which at least one glycan is attached to the nitrogen in the side chain of an asparagine residue. In yet another embodiment, the N-glycosylated IL-2 mutein provided herein comprises two glycans, each of which is attached to the nitrogen in the side chain of an asparagine residue.

In one embodiment, the N-glycosylated IL-2 mutein provided herein comprises three glycans. In one embodiment, the glycan is an N-glycan.

In one embodiment, the N-glycan on the N-glycosylated IL-2 mutein provided herein is oligomannose-type. In another embodiment, the N-glycan on the N-glycosylated IL-2 mutein provided herein is complex-type. In another embodiment, the N-glycan on the N-glycosylated IL-2 mutein provided herein is hydride-type.

In one embodiment, the N-glycan on the N-glycosylated IL-2 mutein provided herein is biantennary complex-type. In another embodiment, the N-glycan on the N-glycosylated IL-2 mutein provided herein is triantennary complex-type. In yet another embodiment, the N-glycan on the N-glycosylated IL-2 mutein provided herein is tetraantennary complex-type.

In one embodiment, the N-glycan on the N-glycosylated IL-2 mutein provided herein is one of the glycans described in Szabo et al., J. Proteome. Res. 2018, 17, 1559-1574, the disclosure of which is incorporated herein by reference in its entirety.

In one embodiment, the IL-2 mutein provided herein comprises: (i) an amino acid substitution at position L18, Q126, or S130; and (ii) amino acid substitutions at positions R38 and F42. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions at positions K32, R38, and F42. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions at positions K32, R38, and F42. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions at positions R38, F42, and K76. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions at positions E15, K32, R38, and F42. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions at positions E15, R38, F42, and K76.

In one embodiment, the IL-2 mutein provided herein comprises: (i) an amino acid substitution at position L18, Q126, or S130; and (ii) amino acid substitutions of R38E and F42A or F42K. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions of K32E, R38E, and F42A or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of K32E, R38E, and F42A or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of R38E, F42A or F42K, and K76E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, K32E, R38E, and F42A or F42K. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38E, F42A or F42K, and K76E.

In one embodiment, the IL-2 mutein provided herein comprises: (i) an amino acid substitution at position L18, Q126, or S130; and (ii) amino acid substitutions at positions K32, R38, and F42. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions of K32E, R38E, and F42A or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of K32E, R38E, and F42A or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of K32E, R38E, and F42A. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of K32E, R38E, and F42K.

In one embodiment, the IL-2 mutein provided herein comprises: (i) an amino acid substitution at position L18, Q126, or S130; and (ii) amino acid substitutions at positions R38, F42, and K76. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions of R38E, F42A or F42K, and K76E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of R38E, F42A or F42K, and K76E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of R38E, F42A, and K76E. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of R38E, F42K, and K76E.

In one embodiment, the IL-2 mutein provided herein comprises: (i) an amino acid substitution at position L18, Q126, or S130; and (ii) amino acid substitutions at positions E15, K32, R38, and F42. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions of E15K, K32E, R38E, and F42A or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, K32E, R38E, and F42A or F42K. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, K32E, R38E, and F42A. In still another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, K32E, R38E, and F42K.

In one embodiment, the IL-2 mutein provided herein comprises: (i) an amino acid substitution at position L18, Q126, or S130; and (ii) amino acid substitutions at positions E15, R38, F42, and K76. In another embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) amino acid substitutions of E15K, R38E, F42A or F42K, and K76E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38E, F42A or F42K, and K76E. In yet another embodiment, the IL-2 mutein provided herein comprises: (i) an amino acid substitution of L18C, Q126E, Q126K, Q126R, Q126S, Q126T, S130A, S130E, S130Q, or S130R; and (ii) amino acid substitutions of E15K, R38E, F42A, and K76E. In still another embodiment, the IL-2 mutein provided herein comprises: (i) an amino acid substitution of L18C, Q126E, Q126K, Q126R, Q126S, Q126T, S130A, S130E, S130Q, or S130R; and (b) amino acid substitutions of E15K, R38E, F42K, and K76E.

In one embodiment, the IL-2 mutein provided herein comprises an amino acid sequence of any one of SEQ ID NOs: 193 to 204, wherein the IL-2 mutein comprises an amino acid substitution of L18C, Q126E, Q126K, Q126R, Q126S, Q126T, S130A, S130E, S130Q, or S130R; wherein the amino acid residue at position T3 is T or A, and the amino acid residue at position C125 is C or S; and wherein, when the IL-2 mutein comprises an amino acid substitution of L18C, the amino acid residue at position C125 is C.

In one embodiment, the IL-2 mutein provided herein comprises: (i) an amino acid substitution at position L18, Q126, or S130; and (ii) a disulfide bond formed between two different amino acid residues from positions N30 to L80. In another embodiment, the IL-2 mutein provided herein comprises: (i) an amino acid substitution at position L18, Q126, or S130; and (ii) a disulfide bond formed between two different amino acid residues from positions K35 to L72. In yet another embodiment, the IL-2 mutein provided herein comprises: (i) an amino acid substitution at position L18, Q126, or S130; and (ii) a disulfide bond formed between two different amino acid residues from positions K35 to L69.

In one embodiment, the IL-2 mutein provided herein comprises: (i) an amino acid substitution at position L18, Q126, or S130; and (ii) a disulfide bond formed between two different amino acid residues, each independently at K35, R38, F42, Y45, E62, V69, or L72. In another embodiment, the IL-2 mutein provided herein comprises: (i) an amino acid substitution at position L18, Q126, or S130; and (ii) a disulfide bond formed between an amino acid residue at position K35, R38, F42, or Y45; and an amino acid residue at position E62, V69, or L72. In yet another embodiment, the IL-2 mutein provided herein comprises: (i) an amino acid substitution at position L18, Q126, or S130; and (ii) a disulfide bond formed between an amino acid residue at position F42 and an amino acid residue at position E62, V69, or L72. In still another embodiment, the IL-2 mutein provided herein comprises: (i) an amino acid substitution at position L18, Q126, or S130; and (ii) a disulfide bond formed between an amino acid residue at position K35, R38, F42, or Y45, and an amino acid residue at position V69.

In one embodiment, the IL-2 mutein provided herein comprises: (i) an amino acid substitution of L18C, L18S, Q126E, Q126K, Q126R, Q126S, Q126T, S130A, S130E, S130Q, or S130R; and (ii) a disulfide bond formed between K35C and L72C, R38C and L72C, F42C and V69C, Y42C and L72C, or Y45C and E62C. In another embodiment, the IL-2 mutein provided herein comprises: (i) an amino acid substitution of L18C, Q126E, Q126K, Q126R, Q126S, Q126T, S130A, S130E, S130Q, or S130R; and (ii) a disulfide bond formed between K35C and L72C, R38C and L72C, F42C and V69C, Y42C and L72C, or Y45C and E62C. In yet another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between K35C and L72C. In yet another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between R38C and L72C. In yet another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between F42C and V69C. In yet another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between Y42C and L72C. In still another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between Y45C and E62C.

In one embodiment, the IL-2 mutein provided herein comprises: (i) an amino acid substitution at position L18, Q126, or S130; and (ii) a disulfide bond formed between two different amino acid residues from positions N30 to L80; and (iii) an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, 587, N88, V91, I92, E95, Y107, D109, T111, or S127.

In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a disulfide bond formed between two different amino acid residues, each independently at position K35, R38, F42, Y45, E62, V69, or L72; and (iii) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, E95K, E95N, E95Q, Y107N, D109N, T111S, S127A, S127E, S127F, or S127W.

In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a disulfide bond formed between two different amino acid residues, each independently at position K35, R38, F42, Y45, E62, V69, or L72; and (iii) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, E95K, E95N, E95Q, Y107N, D109N, T111S, S127A, S127E, S127F, or S127W.

In yet another embodiment, the IL-2 mutein provided herein comprises (i) an amino acid substitution of L18C, L18S, Q126E, Q126K, Q126R, Q126S, Q126T, S130A, S130E, S130Q, or S130R; (ii) a disulfide bond formed between F42C and V69C; and (iii) an amino acid substitution of E15K, H16E, H16F, H16I, H16V, D20A, D20E, D20K, D20T, K32E, K76E, S87D, N88A, I92A, I92D, I92E, or I92G.

In yet another embodiment, the IL-2 mutein provided herein comprises (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of E15K, H16E, H16F, H16I, H16V, D20A, D20E, D20K, D20T, K32E, K76E, S87D, N88A, I92A, I92D, I92E, or I92G.

In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a disulfide bond formed between F42C and V69C; and (iii) an amino acid substitution of E15K, H16E, H16I, H16V, D20A, D20E, D20K, D20T, K32E, K76E, S87D, N88A, I92A, I92D, I92E, or I92G.

In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of E15K, H16E, H16I, H16V, D20A, D20E, D20K, D20T, K32E, K76E, S87D, N88A, I92A, I92D, I92E, or I92G.

In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a disulfide bond formed between F42C and V69C; and (iii) an amino acid substitution of E15K, K32E, or K76E.

In still another embodiment, the IL-2 mutein provided herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of E15K, K32E, or K76E.

In one embodiment, the IL-2 mutein provided herein comprises: (i) an amino acid substitution at position L18, Q126, or S130; and (ii) a disulfide bond formed between F42C and V69C. In another embodiment, the IL-2 mutein provided herein comprises: (i) an amino acid substitution at position L18, Q126, or S130; (ii) a disulfide bond formed between F42C and V69C; an amino acid substitution at position K32 or K76; and (iii) optionally an amino acid substitution at position E15. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of K32E. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of K76E. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) amino acid substitutions of E15K and K32E. In still another embodiment, the IL-2 mutein provided herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) amino acid substitutions of E15K and K76E.

In one embodiment, the IL-2 mutein provided herein comprises an amino acid sequence of any one of SEQ ID NOs: 205 to 216, wherein the IL-2 mutein comprises an amino acid substitution of L18C, Q126E, Q126K, Q126R, Q126S, Q126T, S130A, S130E, S130Q, or S130R; wherein the amino acid residue at position T3 is T or A, and the amino acid residue at position C125 is C or S; and wherein, when the IL-2 mutein comprises an amino acid substitution of L18C, the amino acid residue at position C125 is C.

In one embodiment, the IL-2 mutein provided herein comprises: (i) an amino acid substitution at position L18, Q126, or S130; (ii) a replacement of the amino acid residues from positions N29 to L40 with an IL-15 hinge fragment-containing peptide. In another embodiment, the IL-2 mutein provided herein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219. In yet another embodiment, the IL-2 mutein provided herein comprises a replacement of the amino acid residues from positions N29 to L40 having an amino acid sequence of SEQ ID NO: 217 with an IL-15 hinge fragment-containing peptide having an amino acid sequence of SEQ ID NO: 218. In still another embodiment, the IL-2 mutein provided herein comprises a replacement of the amino acid residues from positions N29 to L40 having an amino acid sequence of SEQ ID NO: 217 with an IL-15 hinge fragment-containing peptide having an amino acid sequence of SEQ ID NO: 219.

In one embodiment, the IL-2 mutein provided herein comprises: (i) an amino acid substitution at position L18, Q126, or S130; (ii) a replacement of the amino acid residues from positions N29 to L40 with an IL-15 hinge fragment-containing peptide; and (iii) an amino acid substitution at position K8, K9, Q13, E15, H16, L19, D20, M23, E61, E62, L63, K64, P65, L66, E67, E68, V69, L70, N71, L72, A73, Q74, K76, H79, R81, D84, S87, N88, V91, I92, E95, Y107, D109, T111, or S127.

In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide; and (iii) an amino acid substitution of K8D, K8E, K8Q, K9D, K9E, K9Q, Q13E, Q13N, E15K, E15Q, E15V, H16D, H16E, H16F, H16I, H16N, H16Q, H16V, L19S, D20A, D20E, D20K, D20T, M23K, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide; and (iii) an amino acid substitution of K8D, K8E, K8Q, K9D, K9E, K9Q, Q13E, Q13N, E15K, E15Q, E15V, H16D, H16E, H16F, H16I, H16N, H16Q, H16V, L19S, D20A, D20E, D20K, D20T, M23K, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In still another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide; and (iii) an amino acid substitution of K8D, K8E, K8Q, K9D, K9E, K9Q, Q13E, Q13N, E15K, E15Q, E15V, H16D, H16E, H16F, H16I, H16N, H16Q, H16V, L19S, D20A, D20E, D20K, D20T, M23K, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In one embodiment, the IL-2 mutein provided herein comprises: (i) an amino acid substitution at position L18, Q126, or S130; (ii) a replacement of the amino acid residues from positions N29 to L40 with an IL-15 hinge fragment-containing peptide; and (iii) an amino acid substitution at position E15, H16, D20, S87, N88, or 192.

In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 219; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 219; and (iii) an amino acid substitution of E15K, H16E, H16F, H16I, H16V, D20A, D20E, D20K, D20T, S87D, N88A, I92A, I92D, I92E, or I92G.

In still another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 219; and (iii) an amino acid substitution of E15K, H16E, H16F, H16I, H16V, D20A, D20E, D20K, D20T, S87D, N88A, I92A, I92D, I92E, or I92G.

In one embodiment, the IL-2 mutein provided herein comprises: (i) an amino acid substitution at position L18, Q126, or S130; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution at E15. In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of E15K. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of E15K.

In one embodiment, the IL-2 mutein provided herein comprises: (i) an amino acid substitution at position L18, Q126, or S130; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of H16. In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of H16E, H16F, H16I, or H16V. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of H16E, H16F, H16I, or H16V.

In one embodiment, the IL-2 mutein provided herein comprises: (i) an amino acid substitution at position L18, Q126, or S130; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of D20. In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of D20A, D20E, D20K, or D20T. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of D20A, D20E, D20K, or D20T.

In one embodiment, the IL-2 mutein provided herein comprises: (i) an amino acid substitution at position L18, Q126, or S130; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of S87. In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of S87D. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of S87D.

In one embodiment, the IL-2 mutein provided herein comprises: (i) an amino acid substitution at position L18, Q126, or S130; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of N88. In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of N88A. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of N88A.

In one embodiment, the IL-2 mutein provided herein comprises: (i) an amino acid substitution at position L18, Q126, or S130; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of 192. In another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92A, I92D, I92E, or I92G. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92A, I92D, I92E, or I92G. In yet another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92A, I92D, I92E, or I92G. In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92A. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92A. In yet another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92A. In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of 192D. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of 192D. In yet another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of 192D. In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92E. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92E. In yet another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92E. In yet another embodiment, the IL-2 mutein provided herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92G. In yet another embodiment, the IL-2 mutein provided herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92G. In still another embodiment, the IL-2 mutein provided herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having an amino acid sequence of SEQ ID NO: 217 with an IL-15 hinge fragment-containing peptide having an amino acid sequence of SEQ ID NO: 218 or 219; and (iii) an amino acid substitution of I92G.

In one embodiment, the IL-2 mutein provided herein comprises an amino acid sequence of any one of SEQ ID NOs: 220 to 261, wherein the IL-2 mutein comprises an amino acid substitution of L18C, L18S, Q126E, Q126K, Q126R, Q126S, Q126T, S130A, S130E, S130Q, or S130R; wherein the amino acid residue at position T3 is T or A, and the amino acid residue at position C125 is C or S; and wherein, when the IL-2 mutein comprises an amino acid substitution of L18C, the amino acid residue at position C125 is C. In another embodiment, the IL-2 mutein provided herein comprises an amino acid sequence of any one of SEQ ID NOs: 220 to 261, wherein the IL-2 mutein comprises an amino acid substitution of L18C, Q126E, Q126K, Q126R, Q126S, Q126T, S130A, S130E, S130Q, or S130R; wherein the amino acid residue at position T3 is T or A, and the amino acid residue at position C125 is C or S; and wherein, when the IL-2 mutein comprises an amino acid substitution of L18C, the amino acid residue at position C125 is C. In yet another embodiment, the IL-2 mutein provided herein comprises an amino acid sequence of any one of SEQ ID NOs: 220 to 261, wherein the IL-2 mutein comprises an amino acid substitution of L18S, Q126E, Q126K, Q126R, Q126S, Q126T, S130A, S130E, S130Q, or S130R; wherein the amino acid residue at position T3 is T or A, and the amino acid residue at position C125 is C or S; and wherein, when the IL-2 mutein comprises an amino acid substitution of L18C, the amino acid residue at position C125 is C. In yet another embodiment, the IL-2 mutein provided herein comprises an amino acid sequence of any one of SEQ ID NOs: 262 to 287. In still another embodiment, the IL-2 mutein provided herein comprises an amino acid sequence of SEQ ID NO: 301.

In certain embodiments, the IL-2 mutein provided herein has an attenuated binding affinity to an IL-2Rα as compared to a wild-type IL-2. In certain embodiments, the binding affinity of the IL-2 mutein provided herein to an IL-2Rα is measured by its Kₐ, which is the inverse of its K_{d}.

In certain embodiments, the IL-2 mutein provided herein has a K_{d} to the IL-2Rα of no less than about 2 times, no less than about 5 times, no less than about 10 times, no less than about 100 times, no less than about 200 times, no less than about 200 times, or no less than about 1,000 times higher than that of the wild-type IL-2 to the IL-2Rα. In one embodiment, the IL-2 mutein provided herein has a K_{d} to the IL-2Rα of no less than about 2 times higher than that of the wild-type IL-2 to the IL-2Rα. In another embodiment, the IL-2 mutein provided herein has a K_{d} to the IL-2Rα of no less than about 5 times higher than that of the wild-type IL-2 to the IL-2Rα. In yet another embodiment, the IL-2 mutein provided herein has a K_{d} to the IL-2Rα of no less than about 10 times higher than that of the wild-type IL-2 to the IL-2Rα. In yet another embodiment, the IL-2 mutein provided herein has a K_{d} to the IL-2Rα of no less than about 100 times higher than that of the wild-type IL-2 to the IL-2Rα. In yet another embodiment, the IL-2 mutein provided herein has a K_{d} to the IL-2Rα of no less than about 200 times higher than that of the wild-type IL-2 to the IL-2Rα. In yet another embodiment, the IL-2 mutein provided herein has a K_{d} to the IL-2Rα of no less than about 500 times higher than that of the wild-type IL-2 to the IL-2Rα. In still another embodiment, the IL-2 mutein provided herein has a K_{d} to the IL-2Rα of no less than about 1,000 times higher than that of the wild-type IL-2 to the IL-2Rα.

In certain embodiments, the IL-2 mutein provided herein has a K_{d} to an IL-2Rα of no less than about 20 nM, no less than about 50 nM, no less than about 100 nM, no less than about 1 μM, no less than about 10 μM, no less than about 100 μM, no less than about 200 μM, no less than about 500 μM, or no less than about 1 mM. In certain embodiments, the IL-2 mutein provided herein has a K_{d} to the IL-2Rα of no less than about 20 nM. In certain embodiments, the IL-2 mutein provided herein has a K_{d} to the IL-2Rα of no less than about 50 nM. In certain embodiments, the IL-2 mutein provided herein has a K_{d} to the IL-2Rα of no less than about 100 nM. In certain embodiments, the IL-2 mutein provided herein has a K_{d} to the IL-2Rα of no less than about 1 μM. In certain embodiments, the IL-2 mutein provided herein has a K_{d} to the IL-2Rα of no less than about 10 μM. In certain embodiments, the IL-2 mutein provided herein has a K_{d} to the IL-2Rα of no less than about 100 μM. In certain embodiments, the IL-2 mutein provided herein has a K_{d} to the IL-2Rα of no less than about 200 μM. In certain embodiments, the IL-2 mutein provided herein has a K_{d} to the IL-2Rα of no less than about 500 μM. In certain embodiments, the IL-2 mutein provided herein has a K_{d} to the IL-2Rα of no less than about 1 mM. In certain embodiments, the IL-2 mutein provided herein has no measurable binding to the IL-2Rα. In certain embodiments, the IL-2 mutein provided herein has no detectable binding to the IL-2Rα as measured with a surface plasmon resonance (SPR) method. In certain embodiments, the IL-2 mutein provided herein has no detectable binding to the IL-2Rα as measured with bio-layer interferometry (BLI).

In certain embodiments, the IL-2 mutein provided herein has a reduced binding affinity to an IL-2Rβ/γ complex as compared to a wild-type IL-2. In certain embodiments, the binding affinity of the IL-2 mutein provided herein to an IL-2Rβ/γ complex is measured by its Kₐ, which is the inverse of its K_{d}.

In certain embodiments, the IL-2 mutein provided herein has a K_{d} to the IL-2Rβ/γ complex of no less than about 5 times, no less than about 10 times, no less than about 20 times, no less than about 50 times, or no less than about 100 times higher than that of the wild-type IL-2 to the IL-2Rβ/γ complex. In one embodiment, the IL-2 mutein provided herein has a K_{d} to the IL-2Rβ/γ complex of no less than about 5 times higher than that of the wild-type IL-2 to the IL-2Rβ/γ complex. In another embodiment, the IL-2 mutein provided herein has a K_{d} to the IL-2Rβ/γ complex of no less than about 10 times higher than that of the wild-type IL-2 to the IL-2Rβ/γ complex. In yet another embodiment, the IL-2 mutein provided herein has a K_{d} to the IL-2Rβ/γ complex of no less than about 20 times higher than that of the wild-type IL-2 to the IL-2Rβ/γ complex. In yet another embodiment, the IL-2 mutein provided herein has a K_{d} to the IL-2Rβ/γ complex of no less than about 50 times higher than that of the wild-type IL-2 to the IL-2Rβ/γ complex. In still another embodiment, the IL-2 mutein provided herein has a K_{d} to the IL-2Rβ/γ complex of no less than about 100 times higher than that of the wild-type IL-2 to the IL-2Rβ/γ complex.

In certain embodiments, the IL-2 mutein provided herein has a K_{d} to an IL-2Rβ/γ complex of no less than about 5 nM, no less than about 10 nM, no less than about 20 nM, no less than about 50 nm, no less than about 100 nM, or no less than about 200 nM. In certain embodiments, the IL-2 mutein provided herein has a K_{d} to the IL-2Rβ/γ complex of no less than about 5 nM. In certain embodiments, the IL-2 mutein provided herein has a K_{d} to the IL-2Rβ/γ complex of no less than about 10 nM. In certain embodiments, the IL-2 mutein provided herein has a K_{d} to the IL-2Rβ/γ complex of no less than about 20 nM. In certain embodiments, the IL-2 mutein provided herein has a K_{d} to the IL-2Rβ/γ complex of no less than about 50 nM. In certain embodiments, the IL-2 mutein provided herein has a K_{d} to the IL-2Rβ/γ complex of no less than about 100 nM. In certain embodiments, the IL-2 mutein provided herein has a K_{d} to the IL-2Rβ/γ complex of no less than about 200 nM.

In one embodiment, the IL-2Rα is a human IL-2Rα. In another embodiment, the human IL-2Rα has an amino acid sequence of SEQ ID NO: 289.

In one embodiment, the IL-2Rβ is a human IL-2Rβ. In another embodiment, the human IL-2Rβ has an amino acid sequence of SEQ ID NO: 290.

In one embodiment, the IL-2Rγ is a human IL-2Rγ. In another embodiment, the human IL-2Rγ has an amino acid sequence of SEQ ID NO: 291.

In one embodiment, a K_{d} of an IL-2 to an IL-2Rα is determined with a surface plasmon resonance (SPR) method. In another embodiment, a K_{d} of an IL-2 to an IL-2Rα is determined with a BIACORE® assay. In yet another embodiment, a K_{d} of an IL-2 to an IL-2Rα is determined with bio-layer interferometry (BLI). In still another embodiment, a K_{d} of an IL-2 to an IL-2Rα is determined with an OCTET® assay.

In one embodiment, a K_{d} of an IL-2 to an IL-2Rβ is determined with an SPR method. In another embodiment, a K_{d} of an IL-2 to an IL-2Rβ is determined with a BIACORE® assay. In yet another embodiment, a K_{d} of an IL-2 to an IL-2Rβ is determined with BLI. In still another embodiment, a K_{d} of an IL-2 to an IL-2Rβ is determined with an OCTET® assay.

In one embodiment, a K_{d} of an IL-2 to an IL-2Rβ/γ complex is determined with an SPR method. In another embodiment, a K_{d} of an IL-2 to an IL-2Rβ/γ complex is determined with a BIACORE® assay. In yet another embodiment, a K_{d} of an IL-2 to an IL-2Rβ/γ complex is determined with BLI. In still another embodiment, a K_{d} of an IL-2 to an IL-2Rβ/γ complex is determined with an OCTET® assay.

In one embodiment, a K_{d} of an IL-2 to an IL-2Rα/β/γ complex is determined with an SPR method. In another embodiment, a K_{d} of an IL-2 to an IL-2Rα/β/γ complex is determined with a BIACORE® assay. In yet another embodiment, a K_{d} of an IL-2 to an IL-2Rα/β/γ complex is determined with BLI. In still another embodiment, a K_{d} of an IL-2 to an IL-2Rα/β/γ complex is determined with an OCTET® assay.

In certain embodiments, the IL-2 mutein provided herein comprises an amino acid sequence that is no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 179, 180, 181, 182, 183, or 184. In certain embodiments, the IL-2 mutein provided herein comprises an amino acid sequence that is no less than about 80% identical to the amino acid sequence of SEQ ID NO: 179, 180, 181, 182, 183, or 184. In certain embodiments, the IL-2 mutein provided herein comprises an amino acid sequence that is no less than about 85% identical to the amino acid sequence of SEQ ID NO: 179, 180, 181, 182, 183, or 184. In certain embodiments, the IL-2 mutein provided herein comprises an amino acid sequence that is no less than about 90% identical to the amino acid sequence of SEQ ID NO: 179, 180, 181, 182, 183, or 184. In certain embodiments, the IL-2 mutein provided herein comprises an amino acid sequence that is no less than about 91% identical to the amino acid sequence of SEQ ID NO: 179, 180, 181, 182, 183, or 184. In certain embodiments, the IL-2 mutein provided herein comprises an amino acid sequence that is no less than about 92% identical to the amino acid sequence of SEQ ID NO: 179, 180, 181, 182, 183, or 184. In certain embodiments, the IL-2 mutein provided herein comprises an amino acid sequence that is no less than about 93% identical to the amino acid sequence of SEQ ID NO: 179, 180, 181, 182, 183, or 184. In certain embodiments, the IL-2 mutein provided herein comprises an amino acid sequence that is no less than about 94% identical to the amino acid sequence of SEQ ID NO: 179, 180, 181, 182, 183, or 184. In certain embodiments, the IL-2 mutein provided herein comprises an amino acid sequence that is no less than about 95% identical to the amino acid sequence of SEQ ID NO: 179, 180, 181, 182, 183, or 184. In certain embodiments, the IL-2 mutein provided herein comprises an amino acid sequence that is no less than about 96% identical to the amino acid sequence of SEQ ID NO: 179, 180, 181, 182, 183, or 184. In certain embodiments, the IL-2 mutein provided herein comprises an amino acid sequence that is no less than about 97% identical to the amino acid sequence of SEQ ID NO: 179, 180, 181, 182, 183, or 184. In certain embodiments, the IL-2 mutein provided herein comprises an amino acid sequence that is no less than about 98% identical to the amino acid sequence of SEQ ID NO: 179, 180, 181, 182, 183, or 184. In certain embodiments, the IL-2 mutein provided herein comprises an amino acid sequence that is no less than about 99% identical to the amino acid sequence of SEQ ID NO: 179, 180, 181, 182, 183, or 184.

In certain embodiments, the IL-2 mutein provided herein comprises one of the amino acid sequences of interleukin-2 variants and muteins described in CN 111018961 A, US 2018/0326010 A1, and WO 2020/005819 A1, the disclosure of each of which is incorporated herein by reference in its entirety; wherein the IL-2 mutein comprises an amino acid substitution at position L18, Q126, or S130.

In certain embodiments, the IL-2 mutein provided herein further includes one or more additional substitutions, deletions, and/or insertions; and/or one or more additional post-translational modifications.

### Fusion Proteins

In one embodiment, provided herein is a fusion protein comprising an IL-2 domain and a half-life-extension domain.

In one embodiment, the half-life-extension domain comprises an albumin binding domain, a constant region of an antibody, a constant region of a heavy chain of an antibody, a fragment crystallizable (Fc) domain, a serum albumin, a polyethylene glycol (PEG) group, or a fatty acyl group. In another embodiment, the half-life-extension domain is an albumin binding domain. In yet another embodiment, the half-life-extension domain is a constant region of an antibody, comprising two light chain constant domains (C_{L}) and two heavy chain constant domains (C_{H}1, C_{H}2, and C_{H}3). In yet another embodiment, the half-life-extension domain is a constant region (C_{H}1, C_{H}2, and C_{H}3) of a heavy chain of an antibody. In yet another embodiment, the half-life-extension domain is an Fc domain. In yet another embodiment, the half-life-extension domain is an Fc domain comprising first and second peptide chains. In yet another embodiment, the half-life-extension domain is a serum albumin. In yet another embodiment, the half-life-extension domain is a PEG group. In still another embodiment, the half-life-extension domain is a fatty acyl group.

In certain embodiments, the half-life-extension domain extends the half-life of the IL-2 domain in vivo as compared to the corresponding free IL-2. In certain embodiments, the half-life-extension domain extends the half-life of the IL-2 domain in vivo as compared to a wild-type IL-2 of SEQ ID NO: 179, 180, 181, 182, 183, or 184. In certain embodiments, the half-life-extension domain extends the half-life of the IL-2 domain in vivo as compared to the corresponding free IL-2.

In one embodiment, provided herein is a fusion protein comprising an IL-2 domain and an albumin binding domain as a half-life-extension domain.

In one embodiment, the fusion protein provided herein comprises an IL-2 domain, an albumin binding domain, and optionally a peptide linker; wherein the carboxy-terminus (C-terminus) of the IL-2 domain is connected to an amino-terminus (N-terminus) of the albumin binding domain directly or via the peptide linker; or wherein a C-terminus of the albumin binding domain is connected to the N-terminus of the IL-2 domain directly or via the peptide linker.

In another embodiment, the fusion protein provided herein comprises an IL-2 domain, an albumin binding domain, and optionally a peptide linker; wherein the C-terminus of the IL-2 domain is connected to an N-terminus of the albumin binding domain directly or via the peptide linker.

In yet another embodiment, the fusion protein provided herein comprises an IL-2 domain, an albumin binding domain, and optionally a peptide linker; wherein a C-terminus of the albumin binding domain is connected to a N-terminus of the IL-2 domain directly or via the peptide linker.

In another embodiment, provided herein is a fusion protein comprising an IL-2 domain, a PD-1 binding domain, and an albumin binding domain as a half-life-extension domain.

In one embodiment, the fusion protein provided herein comprises an IL-2 domain, a PD-1 binding domain, an albumin binding domain, and optionally first and second peptide linkers; wherein a C-terminus of the PD-1 binding domain is connected to an N-terminus of the albumin binding domain directly or via the first peptide linker, and a C-terminus of the albumin binding domain is connected to the N-terminus of the IL-2 domain directly or via the second peptide linker.

In another embodiment, the fusion protein provided herein comprises an IL-2 domain, a PD-1 binding domain, an albumin binding domain, and optionally first and second peptide linkers; wherein the C-terminus of the IL-2 domain is connected to an N-terminus of the albumin binding domain directly or via the first peptide linker, and a C-terminus of the albumin binding domain is connected to an N-terminus of the PD-1 binding domain directly or via the second peptide linker.

In yet another embodiment, the fusion protein provided herein comprises an IL-2 domain, a PD-1 binding domain, an albumin binding domain, and optionally first and second peptide linkers; wherein a C-terminus of the albumin binding domain is connected to an N-terminus of the PD-1 binding domain directly or via the first peptide linker, and an C-terminus of the PD-1 binding domain is connected to the N-terminus of the IL-2 domain directly or via the second peptide linker.

In yet another embodiment, the fusion protein provided herein comprises an IL-2 domain, a PD-1 binding domain, an albumin binding domain, and optionally first and second peptide linkers; wherein the C-terminus of the IL-2 domain is connected to an N-terminus of the PD-1 binding domain directly or via the first peptide linker, and a C-terminus of the PD-1 binding domain is connected to an N-terminus of the albumin binding domain directly or via the second peptide linker.

In yet another embodiment, the fusion protein provided herein comprises an IL-2 domain, a PD-1 binding domain, an albumin binding domain, and optionally first and second peptide linkers; wherein a C-terminus of the albumin binding domain is connected to the N-terminus of the IL-2 domain directly or via the first peptide linker, and the C-terminus of the IL-2 domain is connected to an N-terminus of the PD-1 binding domain directly or via the second peptide linker.

In still another embodiment, the fusion protein provided herein comprises an IL-2 domain, a PD-1 binding domain, an albumin binding domain, and optionally first and second peptide linkers; wherein a C-terminus of the PD-1 binding domain is connected to the N-terminus of the IL-2 domain directly or via the first peptide linker, and the C-terminus of the IL-2 domain is connected to an N-terminus of the albumin binding domain directly or via the second peptide linker.

In one embodiment, the albumin binding domain is an antibody or a fragment thereof that binds to an albumin. In another embodiment, the albumin binding domain is an antibody or a fragment thereof that binds to a human serum albumin (HSA). In yet another embodiment, the albumin binding domain is an antibody or a fragment thereof that binds to an HSA specifically.

In certain embodiments, the anti-HSA antibody binds to an HSA with a K_{d} ranging from about 10 pM to about 1,000 nM, from about 1 to about 500 nM, from about 1 to about 200 nM, or from about 1 to about 100 nM at a pH of about 7. In certain embodiments, the anti-HSA antibody binds to an HSA with a K_{d} ranging from about 10 pM to about 1,000 nM at a pH of about 7. In certain embodiments, the anti-HSA antibody binds to an HSA with a K_{d} ranging from about 1 nM to about 500 nM at a pH of about 7. In certain embodiments, the anti-HSA antibody binds to an HSA with a K_{d} ranging from about 1 nM to about 200 nM at a pH of about 7. In certain embodiments, the anti-HSA antibody binds to an HSA with a K_{d} ranging from about 1 nM to about 100 nM at a pH of about 7.

In one embodiment, the albumin binding domain is an anti-HSA antibody or a fragment thereof, comprising (i) a complementarity determining region 1 (CDR1) of SEQ ID NO: 1, a complementarity determining region 2 (CDR2) of SEQ ID NO: 2, and a complementarity determining region 3 (CDR3) of SEQ ID NO: 3; or (ii) a CDR1 of SEQ ID NO: 9, a CDR2 of SEQ ID NO: 10, and a CDR3 of SEQ ID NO: 11. In another embodiment, the albumin binding domain is an anti-HSA antibody or a fragment thereof, comprising a CDR1 of SEQ ID NO: 1, a CDR2 of SEQ ID NO: 2, and a CDR3 of SEQ ID NO: 3. In yet another embodiment, the albumin binding domain comprises a CDR1 of SEQ ID NO: 9, a CDR2 of SEQ ID NO: 10, and a CDR3 of SEQ ID NO: 11.

In yet another embodiment, the albumin binding domain is an anti-HSA antibody or a fragment thereof, comprising an amino acid sequence of SEQ ID NO: 8 or 15. In yet another embodiment, the albumin binding domain is an anti-HSA antibody or a fragment thereof, comprising an amino acid sequence of SEQ ID NO: 8. In still another embodiment, the albumin binding domain comprises an amino acid sequence of SEQ ID NO: 15.

In certain embodiments, the albumin binding domain is an anti-HSA antibody disclosed in WO 2019/246004 A1 or WO 2020/172528 A1, the disclosure of each of which is incorporated herein by reference in its entirety.

In certain embodiments, the antibody is a human antibody. In certain embodiments, the antibody is a humanized antibody.

In yet another embodiment, provided herein is a fusion protein comprising an IL-2 domain and an anti-HSA antibody.

In one embodiment, the fusion protein provided herein comprises an IL-2 domain, an anti-HSA antibody, and optionally a peptide linker; wherein the C-terminus of the IL-2 domain is connected to an N-terminus of the anti-HSA antibody directly or via the peptide linker; or wherein a C-terminus of the anti-HSA antibody is connected to the N-terminus of the IL-2 domain directly or via the peptide linker.

In another embodiment, the fusion protein provided herein comprises an IL-2 domain, an anti-HSA antibody, and optionally a peptide linker; wherein the C-terminus of the IL-2 domain is connected to an N-terminus of the anti-HSA antibody directly or via the peptide linker.

In yet another embodiment, the fusion protein provided herein comprises an IL-2 domain, an anti-HSA antibody, and optionally a peptide linker; wherein a C-terminus of the anti-HSA antibody is connected to a N-terminus of the IL-2 domain directly or via the peptide linker.

In yet another embodiment, provided herein is a fusion protein comprising an IL-2 domain, a PD-1 binding domain, and an anti-HSA antibody.

In one embodiment, the fusion protein provided herein comprises an IL-2 domain, a PD-1 binding domain, an anti-HSA antibody, and optionally first and second peptide linkers; wherein a C-terminus of the PD-1 binding domain is connected to an N-terminus of the anti-HSA antibody directly or via the first peptide linker, and a C-terminus of the anti-HSA antibody is connected to the N-terminus of the IL-2 domain directly or via the second peptide linker.

In another embodiment, the fusion protein provided herein comprises an IL-2 domain, a PD-1 binding domain, an anti-HSA antibody, and optionally first and second peptide linkers; wherein the C-terminus of the IL-2 domain is connected to an N-terminus of the anti-HSA antibody directly or via the first peptide linker, and a C-terminus of the anti-HSA antibody is connected to an N-terminus of the PD-1 binding domain directly or via the second peptide linker.

In yet another embodiment, the fusion protein provided herein comprises an IL-2 domain, a PD-1 binding domain, an anti-HSA antibody, and optionally first and second peptide linkers; wherein a C-terminus of the anti-HSA antibody is connected to an N-terminus of the PD-1 binding domain directly or via the first peptide linker, and an C-terminus of the PD-1 binding domain is connected to the N-terminus of the IL-2 domain directly or via the second peptide linker.

In yet another embodiment, the fusion protein provided herein comprises an IL-2 domain, a PD-1 binding domain, an anti-HSA antibody, and optionally first and second peptide linkers; wherein the C-terminus of the IL-2 domain is connected to an N-terminus of the PD-1 binding domain directly or via the first peptide linker, and a C-terminus of the PD-1 binding domain is connected to an N-terminus of the anti-HSA antibody directly or via the second peptide linker.

In yet another embodiment, the fusion protein provided herein comprises an IL-2 domain, a PD-1 binding domain, an anti-HSA antibody, and optionally first and second peptide linkers; wherein a C-terminus of the anti-HSA antibody is connected to the N-terminus of the IL-2 domain directly or via the first peptide linker, and the C-terminus of the IL-2 domain is connected to an N-terminus of the PD-1 binding domain directly or via the second peptide linker.

In still another embodiment, the fusion protein provided herein comprises an IL-2 domain, a PD-1 binding domain, an anti-HSA antibody, and optionally first and second peptide linkers; wherein a C-terminus of the PD-1 binding domain is connected to the N-terminus of the IL-2 domain directly or via the first peptide linker, and the C-terminus of the IL-2 domain is connected to an N-terminus of the anti-HSA antibody directly or via the second peptide linker.

In certain embodiments, the anti-HSA antibody is an anti-HSA single domain antibody (sdAb).

In certain embodiments, the anti-HSA sdAb binds to an HSA with a K_{d} ranging from about 10 pM to about 1,000 nM, from about 1 to about 500 nM, from about 1 to about 200 nM, or from about 1 to about 100 nM at a pH of about 7. In certain embodiments, the anti-HSA sdAb binds to an HSA with a K_{d} ranging from about 10 pM to about 1,000 nM at a pH of about 7. In certain embodiments, the anti-HSA sdAb binds to an HSA with a K_{d} ranging from about 1 to about 500 nM at a pH of about 7. In certain embodiments, the anti-HSA sdAb binds to an HSA with a K_{d} ranging from about 1 to about 200 nM at a pH of about 7. In certain embodiments, the anti-HSA sdAb binds to an HSA with a K_{d} ranging from about 1 to about 100 nM at a pH of about 7.

In one embodiment, the anti-HSA sdAb comprises (i) a CDR1 of SEQ ID NO: 1, a CDR2 of SEQ ID NO: 2, and a CDR3 of SEQ ID NO: 3; or (ii) a CDR1 of SEQ ID NO: 9, a CDR2 of SEQ ID NO: 10, and a CDR3 of SEQ ID NO: 11. In another embodiment, the anti-HSA sdAb comprises a CDR1 of SEQ ID NO: 1, a CDR2 of SEQ ID NO: 2, and a CDR3 of SEQ ID NO: 3. In yet another embodiment, the anti-HSA sdAb comprises a CDR1 of SEQ ID NO: 9, a CDR2 of SEQ ID NO: 10, and a CDR3 of SEQ ID NO: 11.

In one embodiment, the anti-HSA sdAb has the structure of FR1-CDR1-FR2-CDR2-FR3-CDR3-FR4, wherein:
CDR1, CDR2, and CDR3 are:
   (i) a CDR1 of SEQ ID NO: 1, a CDR2 of SEQ ID NO: 2, and a CDR3 of SEQ ID NO: 3; or
   (ii) a CDR1 of SEQ ID NO: 9, a CDR2 of SEQ ID NO: 10, and a CDR3 of SEQ ID NO: 11;
FR1 is an amino acid sequence of SEQ ID NO: 4 or 12;
FR2 is an amino acid sequence of SEQ ID NO: 5 or 13;
FR3 is an amino acid sequence of SEQ ID NO: 6; and
FR4 is an amino acid sequence of SEQ ID NO: 7 or 14.

In another embodiment, the anti-HSA sdAb has the structure of FR1-CDR1-FR2-CDR2-FR3-CDR3-FR4, wherein:
CDR1, CDR2, and CDR3 are:
   (i) a CDR1 of SEQ ID NO: 1, a CDR2 of SEQ ID NO: 2, and a CDR3 of SEQ ID NO: 3; or
   (ii) a CDR1 of SEQ ID NO: 9, a CDR2 of SEQ ID NO: 10, and a CDR3 of SEQ ID NO: 11;
FR1 is an amino acid sequence of SEQ ID NO: 4;
FR2 is an amino acid sequence of SEQ ID NO: 5;
FR3 is an amino acid sequence of SEQ ID NO: 6; and
FR3 is an amino acid sequence of SEQ ID NO: 7.

In yet another embodiment, the anti-HSA sdAb has the structure of FR1-CDR1-FR2-CDR2-FR3-CDR3-FR4, wherein:
CDR1, CDR2, and CDR3 are:
   (i) a CDR1 of SEQ ID NO: 1, a CDR2 of SEQ ID NO: 2, and a CDR3 of SEQ ID NO: 3; or
   (ii) a CDR1 of SEQ ID NO: 9, a CDR2 of SEQ ID NO: 10, and a CDR3 of SEQ ID NO: 11;
FR1 is an amino acid sequence of SEQ ID NO: 12;
FR2 is an amino acid sequence of SEQ ID NO: 13;
FR3 is an amino acid sequence of SEQ ID NO: 6; and
FR3 is an amino acid sequence of SEQ ID NO: 14.

In one embodiment, the anti-HSA sdAb has an amino acid sequence of SEQ ID NO: 8 or 15. In another embodiment, the anti-HSA sdAb has an amino acid sequence of SEQ ID NO: 8. In yet another embodiment, the anti-HSA sdAb has an amino acid sequence of SEQ ID NO: 15.

In yet another embodiment, provided herein is a fusion protein comprising an IL-2 domain and an anti-HSA sdAb.

In one embodiment, the fusion protein provided herein comprises an IL-2 domain, an anti-HSA sdAb, and optionally a peptide linker; wherein the C-terminus of the IL-2 domain is connected to the N-terminus of the anti-HSA sdAb directly or via the peptide linker; or wherein the C-terminus of the anti-HSA sdAb is connected to the N-terminus of the IL-2 domain directly or via the peptide linker.

In another embodiment, the fusion protein provided herein comprises an IL-2 domain, an anti-HSA sdAb, and optionally a peptide linker; wherein the C-terminus of the IL-2 domain is connected to the N-terminus of the anti-HSA sdAb directly or via the peptide linker.

In yet another embodiment, the fusion protein provided herein comprises an IL-2 domain, an anti-HSA sdAb, and optionally a peptide linker; wherein the C-terminus of the anti-HSA sdAb is connected to a N-terminus of the IL-2 domain directly or via the peptide linker.

In yet another embodiment, provided herein is a fusion protein comprising an IL-2 domain, a PD-1 binding domain, and an anti-HSA sdAb.

In one embodiment, the fusion protein provided herein comprises an IL-2 domain, a PD-1 binding domain, an anti-HSA sdAb, and optionally first and second peptide linkers; wherein a C-terminus of the PD-1 binding domain is connected to the N-terminus of the anti-HSA sdAb directly or via the first peptide linker, and the C-terminus of the anti-HSA sdAb is connected to the N-terminus of the IL-2 domain directly or via the second peptide linker.

In another embodiment, the fusion protein provided herein comprises an IL-2 domain, a PD-1 binding domain, an anti-HSA sdAb, and optionally first and second peptide linkers; wherein the C-terminus of the IL-2 domain is connected to the N-terminus of the anti-HSA sdAb directly or via the first peptide linker, and the C-terminus of the anti-HSA sdAb is connected to an N-terminus of the PD-1 binding domain directly or via the second peptide linker.

In yet another embodiment, the fusion protein provided herein comprises an IL-2 domain, a PD-1 binding domain, an anti-HSA sdAb, and optionally first and second peptide linkers; wherein the C-terminus of the anti-HSA sdAb is connected to a N-terminus of the PD-1 binding domain directly or via the first peptide linker, and an C-terminus of the PD-1 binding domain is connected to the N-terminus of the IL-2 domain directly or via the second peptide linker.

In yet another embodiment, the fusion protein provided herein comprises an IL-2 domain, a PD-1 binding domain, an anti-HSA sdAb, and optionally first and second peptide linkers; wherein the C-terminus of the IL-2 domain is connected to an N-terminus of the PD-1 binding domain directly or via the first peptide linker, and a C-terminus of the PD-1 binding domain is connected to the N-terminus of the anti-HSA sdAb directly or via the second peptide linker.

In yet another embodiment, the fusion protein provided herein comprises an IL-2 domain, a PD-1 binding domain, an anti-HSA sdAb, and optionally first and second peptide linkers; wherein the C-terminus of the anti-HSA sdAb is connected to the N-terminus of the IL-2 domain directly or via the first peptide linker, and the C-terminus of the IL-2 domain is connected to an N-terminus of the PD-1 binding domain directly or via the second peptide linker.

In still another embodiment, the fusion protein provided herein comprises an IL-2 domain, a PD-1 binding domain, an anti-HSA sdAb, and optionally first and second peptide linkers; wherein a C-terminus of the PD-1 binding domain is connected to the N-terminus of the IL-2 domain directly or via the first peptide linker, and the C-terminus of the IL-2 domain is connected to the N-terminus of the anti-HSA sdAb directly or via the second peptide linker.

In certain embodiments, the anti-HSA antibody is an anti-HSA V_{H}H sdAb.

In certain embodiments, the anti-HSA V_{H}H sdAb binds to an HSA with a K_{d} ranging from about 10 pM to about 1,000 nM, from about 1 to about 500 nM, from about 1 to about 200 nM, or from about 1 to about 100 nM at a pH of about 7. In certain embodiments, the anti-HSA V_{H}H sdAb binds to an HSA with a K_{d} ranging from about 10 pM to about 1,000 nM at a pH of about 7. In certain embodiments, the anti-HSA V_{H}H sdAb binds to an HSA with a K_{d} ranging from about 1 to about 500 nM at a pH of about 7. In certain embodiments, the anti-HSA V_{H}H sdAb binds to an HSA with a K_{d} ranging from about 1 to about 200 nM at a pH of about 7. In certain embodiments, the anti-HSA V_{H}H sdAb binds to an HSA with a K_{d} ranging from about 1 to about 100 nM at a pH of about 7.

In one embodiment, the anti-HSA V_{H}H sdAb comprises (i) a heavy chain CDR1 of SEQ ID NO: 1, a heavy chain CDR2 of SEQ ID NO: 2, and a heavy chain CDR3 of SEQ ID NO: 3; or (ii) a heavy chain CDR1 of SEQ ID NO: 9, a heavy chain CDR2 of SEQ ID NO: 10, and a heavy chain CDR3 of SEQ ID NO: 11. In another embodiment, the anti-HSA V_{H}H sdAb comprises a heavy chain CDR1 of SEQ ID NO: 1, a heavy chain CDR2 of SEQ ID NO: 2, and a heavy chain CDR3 of SEQ ID NO: 3. In yet another embodiment, the anti-HSA V_{H}H sdAb comprises a heavy chain CDR1 of SEQ ID NO: 9, a heavy chain CDR2 of SEQ ID NO: 10, and a heavy chain CDR3 of SEQ ID NO: 11.

In one embodiment, the anti-HSA V_{H}H sdAb has the structure of FR1-CDR1-FR2-CDR2-FR3-CDR3-FR4, wherein:
CDR1, CDR2, and CDR3 are:
   (i) a CDR1 of SEQ ID NO: 1, a CDR2 of SEQ ID NO: 2, and a CDR3 of SEQ ID NO: 3; or
   (ii) a CDR1 of SEQ ID NO: 9, a CDR2 of SEQ ID NO: 10, and a CDR3 of SEQ ID NO: 11;
FR1 is an amino acid sequence of SEQ ID NO: 4 or 12;
FR2 is an amino acid sequence of SEQ ID NO: 5 or 13;
FR3 is an amino acid sequence of SEQ ID NO: 6; and
FR4 is an amino acid sequence of SEQ ID NO: 7 or 14.

In another embodiment, the anti-HSA V_{H}H sdAb has the structure of FR1-CDR1-FR2-CDR2-FR3-CDR3-FR4, wherein:
CDR1, CDR2, and CDR3 are:
(i) a CDR1 of SEQ ID NO: 1, a CDR2 of SEQ ID NO: 2, and a CDR3 of SEQ ID NO: 3; or
(ii) a CDR1 of SEQ ID NO: 9, a CDR2 of SEQ ID NO: 10, and a CDR3 of SEQ ID NO: 11;
FR1 is an amino acid sequence of SEQ ID NO: 4;
FR2 is an amino acid sequence of SEQ ID NO: 5;
FR3 is an amino acid sequence of SEQ ID NO: 6; and
FR3 is an amino acid sequence of SEQ ID NO: 7.

In yet another embodiment, the anti-HSA V_{H}H sdAb has the structure of FR1-CDR1-FR2-CDR2-FR3-CDR3-FR4, wherein:
CDR1, CDR2, and CDR3 are:
(i) a CDR1 of SEQ ID NO: 1, a CDR2 of SEQ ID NO: 2, and a CDR3 of SEQ ID NO: 3; or
(ii) a CDR1 of SEQ ID NO: 9, a CDR2 of SEQ ID NO: 10, and a CDR3 of SEQ ID NO: 11;
FR1 is an amino acid sequence of SEQ ID NO: 12;
FR2 is an amino acid sequence of SEQ ID NO: 13;
FR3 is an amino acid sequence of SEQ ID NO: 6; and
FR3 is an amino acid sequence of SEQ ID NO: 14.

In one embodiment, the anti-HSA V_{H}H sdAb has an amino acid sequence of SEQ ID NO: 8 or 15. In another embodiment, the anti-HSA V_{H}H sdAb has an amino acid sequence of SEQ ID NO: 8. In yet another embodiment, the anti-HSA V_{H}H sdAb has an amino acid sequence of SEQ ID NO: 15.

In certain embodiments, the anti-HSA V_{H}H sdAb is a human antibody. In certain embodiments, the anti-HSA V_{H}H sdAb is a humanized antibody.

In yet another embodiment, provided herein is a fusion protein comprising an IL-2 domain and an anti-HSA V_{H}H sdAb.

In one embodiment, the fusion protein provided herein comprises an IL-2 domain, an anti-HSA V_{H}H sdAb, and optionally a peptide linker; wherein the C-terminus of the IL-2 domain is connected to the N-terminus of the anti-HSA V_{H}H sdAb directly or via the peptide linker; or wherein the C-terminus of the anti-HSA V_{H}H sdAb is connected to the N-terminus of the IL-2 domain directly or via the peptide linker.

In another embodiment, the fusion protein provided herein comprises an IL-2 domain, an anti-HSA V_{H}H sdAb, and optionally a peptide linker; wherein the C-terminus of the IL-2 domain is connected to the N-terminus of the anti-HSA V_{H}H sdAb directly or via the peptide linker.

In yet another embodiment, the fusion protein provided herein comprises an IL-2 domain, an anti-HSA V_{H}H sdAb, and optionally a peptide linker; wherein the C-terminus of the anti-HSA V_{H}H sdAb is connected to a N-terminus of the IL-2 domain directly or via the peptide linker.

In yet another embodiment, provided herein is a fusion protein comprising an IL-2 domain, a PD-1 binding domain, and an anti-HSA V_{H}H sdAb.

In one embodiment, the fusion protein provided herein comprises an IL-2 domain, a PD-1 binding domain, an anti-HSA V_{H}H sdAb, and optionally first and second peptide linkers; wherein a C-terminus of the PD-1 binding domain is connected to the N-terminus of the anti-HSA V_{H}H sdAb directly or via the first peptide linker, and the C-terminus of the anti-HSA V_{H}H sdAb is connected to the N-terminus of the IL-2 domain directly or via the second peptide linker.

In another embodiment, the fusion protein provided herein comprises an IL-2 domain, a PD-1 binding domain, an anti-HSA V_{H}H sdAb, and optionally first and second peptide linkers; wherein the C-terminus of the IL-2 domain is connected to the N-terminus of the anti-HSA V_{H}H sdAb directly or via the first peptide linker, and the C-terminus of the anti-HSA V_{H}H sdAb is connected to an N-terminus of the PD-1 binding domain directly or via the second peptide linker.

In yet another embodiment, the fusion protein provided herein comprises an IL-2 domain, a PD-1 binding domain, an anti-HSA V_{H}H sdAb, and optionally first and second peptide linkers; wherein the C-terminus of the anti-HSA V_{H}H sdAb is connected to a N-terminus of the PD-1 binding domain directly or via the first peptide linker, and an C-terminus of the PD-1 binding domain is connected to the N-terminus of the IL-2 domain directly or via the second peptide linker.

In yet another embodiment, the fusion protein provided herein comprises an IL-2 domain, a PD-1 binding domain, an anti-HSA V_{H}H sdAb, and optionally first and second peptide linkers; wherein the C-terminus of the IL-2 domain is connected to an N-terminus of the PD-1 binding domain directly or via the first peptide linker, and a C-terminus of the PD-1 binding domain is connected to the N-terminus of the anti-HSA V_{H}H sdAb directly or via the second peptide linker.

In yet another embodiment, the fusion protein provided herein comprises an IL-2 domain, a PD-1 binding domain, an anti-HSA V_{H}H sdAb, and optionally first and second peptide linkers; wherein the C-terminus of the anti-HSA V_{H}H sdAb is connected to the N-terminus of the IL-2 domain directly or via the first peptide linker, and the C-terminus of the IL-2 domain is connected to an N-terminus of the PD-1 binding domain directly or via the second peptide linker.

In still another embodiment, the fusion protein provided herein comprises an IL-2 domain, a PD-1 binding domain, an anti-HSA V_{H}H sdAb, and optionally first and second peptide linkers; wherein a C-terminus of the PD-1 binding domain is connected to the N-terminus of the IL-2 domain directly or via the first peptide linker, and the C-terminus of the IL-2 domain is connected to the N-terminus of the anti-HSA V_{H}H sdAb directly or via the second peptide linker.

In yet another embodiment, provided herein is a fusion protein comprising an IL-2 domain and an Fc domain as a half-life-extension domain.

In one embodiment, the fusion protein provided herein comprises an IL-2 domain, an Fc domain comprising first and second peptide chains, and optionally a peptide linker; wherein the C-terminus of the IL-2 domain is connected to the N-terminus of the first peptide chain of the Fc domain directly or via the peptide linker, or the C-terminus of the first peptide chain of the Fc domain is connected to the N-terminus of the IL-2 domain directly or via the peptide linker.

In another embodiment, the fusion protein provided herein comprises an IL-2 domain, an Fc domain comprising first and second peptide chains, and optionally a peptide linker; wherein the C-terminus of the IL-2 domain is connected to the N-terminus of the first peptide chain of the Fc domain directly or via the peptide linker.

In yet another embodiment, the fusion protein provided herein comprises an IL-2 domain, an Fc domain comprising first and second peptide chains, and optionally a peptide linker; wherein the C-terminus of the first peptide chain of the Fc domain is connected to the N-terminus of the IL-2 domain directly or via the peptide linker.

In yet another embodiment, provided herein is a fusion protein comprising two IL-2 domains and an Fc domain.

In one embodiment, the fusion protein provided herein comprises first and second IL-2 domains, an Fc domain comprising first and second peptide chains, and optionally first and second peptide linkers; wherein the C-terminus of the first IL-2 domain is connected to the N-terminus of the first peptide chain of the Fc domain directly or via the first peptide linker, and the C-terminus of the second IL-2 domain is connected to the N-terminus of the second peptide chain of the Fc domain directly or via the second peptide linker.

In another embodiment, the fusion protein provided herein comprises first and second IL-2 domains, an Fc domain comprising first and second peptide chains, and optionally first and second peptide linkers; wherein the C-terminus of the first peptide chain of the Fc domain is connected to the N-terminus of the first IL-2 domain directly or via the first peptide linker, and the C-terminus of the second IL-2 domain is connected to the N-terminus of the second peptide chain of the Fc domain directly or via the second peptide linker.

In yet another embodiment, the fusion protein provided herein comprises first and second IL-2 domains, an Fc domain comprising first and second peptide chains, and optionally first and second peptide linkers; wherein the C-terminus of the first peptide chain of the Fc domain is connected to the N-terminus of the first IL-2 domain directly or via the first peptide linker, and the C-terminus of the second peptide chain of the Fc domain is connected to the N-terminus of the second IL-2 domain directly or via the second peptide linker.

In yet another embodiment, provided herein is a fusion protein comprising an IL-2 domain, a PD-1 binding domain, and an Fc domain as a half-life-extension domain.

In yet another embodiment, provided herein is a fusion protein comprising an IL-2 domain, two PD-1 binding domains, and an Fc domain as a half-life-extension domain.

In one embodiment, the fusion protein provided herein comprises an IL-2 domain, first and second PD-1 binding domains, an Fc domain comprising first and second peptide chains, and optionally a peptide linker; wherein a C-terminus of the first PD-1 binding domain is connected to the N-terminus of the first peptide chain of the Fc domain directly or via a peptide linker, and the C-terminus of the first peptide chain of the Fc domain is connected to the N-terminus of the IL-2 domain directly or via the peptide linker; and wherein a C-terminus of the second PD-1 binding domain is connected to the N-terminus of the second peptide chain of the Fc domain directly or via a peptide linker.

In yet another embodiment, provided herein is a fusion protein comprising two IL-2 domain, two PD-1 binding domains, and an Fc domain as a half-life-extension domain.

In one embodiment, the fusion protein provided herein comprises first and second IL-2 domains, first and second PD-1 binding domains, an Fc domain comprising first and second peptide chains, and optionally a peptide linker; wherein a C-terminus of the first PD-1 binding domain is connected to the N-terminus of the first peptide chain of the Fc domain directly or via a peptide linker, and the C-terminus of the first peptide chain of the Fc domain is connected to the N-terminus of the first IL-2 domain directly or via the first peptide linker; and wherein a C-terminus of the second PD-1 binding domain is connected to the N-terminus of the second peptide chain of the Fc domain directly or via a peptide linker, and the C-terminus of the second peptide chain of the Fc domain is connected to the N-terminus of the second IL-2 domain directly or via the second peptide linker.

In one embodiment, the PD-1 binding domain is a single-chain variable fragment (scFv), Fab, Fab′, F(ab)₂, F(ab′)₂, Fv, diabody, triabody, tetrabody, minibody, or a V_{H}H sdAb. In another embodiment, the PD-1 binding domain is an anti-PD-1 scFv. In yet another embodiment, the PD-1 binding domain is an anti-PD-1 Fab. In yet another embodiment, the PD-1 binding domain is an anti-PD-1 Fab′. In yet another embodiment, the PD-1 binding domain is an anti-PD-1 F(ab)₂. In yet another embodiment, the PD-1 binding domain is an anti-PD-1 F(ab′)₂. In yet another embodiment, the PD-1 binding domain is an anti-PD-1 Fv. In yet another embodiment, the PD-1 binding domain is an anti-PD-1 diabody. In yet another embodiment, the PD-1 binding domain is an anti-PD-1 triabody. In yet another embodiment, the PD-1 binding domain is an anti-PD-1 tetrabody. In yet another embodiment, the PD-1 binding domain is an anti-PD-1 minibody. In yet another embodiment, the PD-1 binding domain is an anti-PD-1 sdAb. In still another embodiment, the PD-1 binding domain is an anti-PD-1 V_{H}H sdAb.

In one embodiment, the PD-1 binding domain is an anti-PD-1 sdAb.

Thus, in one embodiment, provided herein is a fusion protein comprising an IL-2 domain, an anti-PD-1 sdAb, and an Fc domain. In another embodiment, provided herein is a fusion protein comprising an IL-2 domain, two anti-PD-1 sdAbs, and an Fc domain.

In one embodiment, the fusion protein provided herein comprises an IL-2 domain, first and second anti-PD-1 sdAbs, an Fc domain comprising a first and second peptide chains, and optionally a peptide linker; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the IL-2 domain directly or via the peptide linker; and wherein the C-terminus of the second anti-PD-1 sdAb is connected to the N-terminus of the second peptide chain of the Fc domain directly or via a peptide linker.

In another embodiment, the fusion protein provided herein comprises an IL-2 domain, first and second anti-PD-1 sdAbs, an Fc domain comprising a first and second peptide chains, and optionally a peptide linker; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the IL-2 domain via the peptide linker; and wherein the C-terminus of the second anti-PD-1 sdAb is connected to the N-terminus of the second peptide chain of the Fc domain directly or via a peptide linker.

In another embodiment, provided herein is a fusion protein comprising two IL-2 domains, two anti-PD-1 sdAbs, and an Fc domain.

In one embodiment, the fusion protein provided herein comprises first and second IL-2 domains, first and second anti-PD-1 sdAbs, an Fc domain comprising a first and second peptide chains, and optionally first and second peptide linker; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the first IL-2 domain directly or via the first peptide linker; and wherein the C-terminus of the second anti-PD-1 sdAb is connected to the N-terminus of the second peptide chain of the Fc domain directly or via a peptide linker, and the C-terminus of the second peptide chain of the Fc domain is connected to the N-terminus of the second IL-2 domain directly or via the second peptide linker.

In another embodiment, the fusion protein provided herein comprises first and second IL-2 domains, first and second anti-PD-1 sdAbs, an Fc domain comprising a first and second peptide chains, and optionally first and second peptide linker; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the first IL-2 domain via the first peptide linker; and wherein the C-terminus of the second anti-PD-1 sdAb is connected to the N-terminus of the second peptide chain of the Fc domain directly or via a peptide linker, and the C-terminus of the second peptide chain of the Fc domain is connected to the N-terminus of the second IL-2 domain via the second peptide linker.

In another embodiment, the PD-1 binding domain is an anti-PD-1 V_{H}H sdAb.

Thus, in one embodiment, provided herein is a fusion protein comprising an IL-2 domain, an anti-PD-1 V_{H}H sdAb, and an Fc domain. In another embodiment, provided herein is a fusion protein comprising an IL-2 domain, two anti-PD-1 V_{H}H sdAbs, and an Fc domain.

In one embodiment, the fusion protein provided herein comprises an IL-2 domain, first and second anti-PD-1 V_{H}H sdAbs, an Fc domain comprising a first and second peptide chains, and optionally a peptide linker; wherein the C-terminus of the first anti-PD-1 V_{H}H sdAb is connected to the N-terminus of the first peptide chain of the Fc domain directly or via a peptide linker, and the C-terminus of the first peptide chain of the Fc domain is connected to the N-terminus of the IL-2 domain directly or via the peptide linker; and wherein the C-terminus of the second anti-PD-1 V_{H}H sdAb is connected to the N-terminus of the second peptide chain of the Fc domain directly or via a peptide linker.

In another embodiment, the fusion protein provided herein comprises an IL-2 domain, first and second anti-PD-1 V_{H}H sdAbs, an Fc domain comprising a first and second peptide chains, and optionally a peptide linker; wherein the C-terminus of the first anti-PD-1 V_{H}H sdAb is connected to the N-terminus of the first peptide chain of the Fc domain directly or via a peptide linker, and the C-terminus of the first peptide chain of the Fc domain is connected to the N-terminus of the IL-2 domain via the peptide linker; and wherein the C-terminus of the second anti-PD-1 V_{H}H sdAb is connected to the N-terminus of the second peptide chain of the Fc domain directly or via a peptide linker.

In another embodiment, provided herein is a fusion protein comprising two IL-2 domains, two anti-PD-1 V_{H}H sdAbs, and an Fc domain.

In one embodiment, the fusion protein provided herein comprises first and second IL-2 domains, first and second anti-PD-1 V_{H}H sdAbs, an Fc domain comprising a first and second peptide chains, and optionally first and second peptide linker; wherein the C-terminus of the first anti-PD-1 V_{H}H sdAb is connected to the N-terminus of the first peptide chain of the Fc domain directly or via a peptide linker, and the C-terminus of the first peptide chain of the Fc domain is connected to the N-terminus of the first IL-2 domain directly or via the first peptide linker; and wherein the C-terminus of the second anti-PD-1 V_{H}H sdAb is connected to the N-terminus of the second peptide chain of the Fc domain directly or via a peptide linker, and the C-terminus of the second peptide chain of the Fc domain is connected to the N-terminus of the second IL-2 domain directly or via the second peptide linker.

In another embodiment, the fusion protein provided herein comprises first and second IL-2 domains, first and second anti-PD-1 V_{H}H sdAbs, an Fc domain comprising a first and second peptide chains, and optionally first and second peptide linker; wherein the C-terminus of the first anti-PD-1 V_{H}H sdAb is connected to the N-terminus of the first peptide chain of the Fc domain directly or via a peptide linker, and the C-terminus of the first peptide chain of the Fc domain is connected to the N-terminus of the first IL-2 domain via the first peptide linker; and wherein the C-terminus of the second anti-PD-1 V_{H}H sdAb is connected to the N-terminus of the second peptide chain of the Fc domain directly or via a peptide linker, and the C-terminus of the second peptide chain of the Fc domain is connected to the N-terminus of the second IL-2 domain via the second peptide linker.

In one embodiment, each anti-PD-1 V_{H}H sdAb independently comprises (i) a CDR1 of SEQ ID NO: 119, a CDR2 of SEQ ID NO: 120, and a CDR3 of SEQ ID NO: 121; (ii) a CDR1 of SEQ ID NO: 123, a CDR2 of SEQ ID NO: 124, and a CDR3 of SEQ ID NO: 125; (iii) a CDR1 of SEQ ID NO: 127, a CDR2 of SEQ ID NO: 128, and a CDR3 of SEQ ID NO: 129; (iv) a CDR1 of SEQ ID NO: 131, a CDR2 of SEQ ID NO: 132, and a CDR3 of SEQ ID NO: 133; or (v) a CDR1 of SEQ ID NO: 135, a CDR2 of SEQ ID NO: 136, and a CDR3 of SEQ ID NO: 137. In another embodiment, each anti-PD-1 V_{H}H sdAb comprises a CDR1 of SEQ ID NO: 119, a CDR2 of SEQ ID NO: 120, and a CDR3 of SEQ ID NO: 121. In yet another embodiment, each anti-PD-1 V_{H}H single domain antibody comprises a CDR1 of SEQ ID NO: 123, a CDR2 of SEQ ID NO: 124, and a CDR3 of SEQ ID NO: 125. In yet another embodiment, each anti-PD-1 V_{H}H single domain antibody comprises a CDR1 of SEQ ID NO: 127, a CDR2 of SEQ ID NO: 128, and a CDR3 of SEQ ID NO: 129. In yet another embodiment, each anti-PD-1 V_{H}H single domain antibody comprises a CDR1 of SEQ ID NO: 131, a CDR2 of SEQ ID NO: 132, and a CDR3 of SEQ ID NO: 133. In still another embodiment, each anti-PD-1 V_{H}H single domain antibody comprises a CDR1 of SEQ ID NO: 135, a CDR2 of SEQ ID NO: 136, and a CDR3 of SEQ ID NO: 137.

In yet another embodiment, the PD-1 binding domain is an anti-PD-1 scFv.

Thus, in one embodiment, provided herein is a fusion protein comprising an IL-2 domain, an anti-PD-1 scFv, and an Fc domain. In another embodiment, provided herein is a fusion protein comprising an IL-2 domain, two anti-PD-1 scFvs, and an Fc domain.

In one embodiment, the fusion protein provided herein comprises an IL-2 domain, first and second anti-PD-1 scFvs, an Fc domain comprising a first and second peptide chains, and optionally a peptide linker; wherein the C-terminus of the first anti-PD-1 scFv is connected to the N-terminus of the first peptide chain of the Fc domain directly or via a peptide linker, and the C-terminus of the first peptide chain of the Fc domain is connected to the N-terminus of the IL-2 domain directly or via the peptide linker; and wherein the C-terminus of the second anti-PD-1 scFv is connected to the N-terminus of the second peptide chain of the Fc domain directly or via a peptide linker.

In another embodiment, the fusion protein provided herein comprises an IL-2 domain, first and second anti-PD-1 scFvs, an Fc domain comprising a first and second peptide chains, and optionally a peptide linker; wherein the C-terminus of the first anti-PD-1 scFv is connected to the N-terminus of the first peptide chain of the Fc domain directly or via a peptide linker, and the C-terminus of the first peptide chain of the Fc domain is connected to the N-terminus of the IL-2 domain via the peptide linker; and wherein the C-terminus of the second anti-PD-1 scFv is connected to the N-terminus of the second peptide chain of the Fc domain directly or via a peptide linker.

In another embodiment, provided herein is a fusion protein comprising two IL-2 domains, two anti-PD-1 scFvs, and an Fc domain.

In one embodiment, the fusion protein provided herein comprises first and second IL-2 domains, first and second anti-PD-1 scFvs, an Fc domain comprising a first and second peptide chains, and optionally first and second peptide linker; wherein the C-terminus of the first anti-PD-1 scFv is connected to the N-terminus of the first peptide chain of the Fc domain directly or via a peptide linker, and the C-terminus of the first peptide chain of the Fc domain is connected to the N-terminus of the first IL-2 domain directly or via the first peptide linker; and wherein the C-terminus of the second anti-PD-1 scFv is connected to the N-terminus of the second peptide chain of the Fc domain directly or via a peptide linker, and the C-terminus of the second peptide chain of the Fc domain is connected to the N-terminus of the second IL-2 domain directly or via the second peptide linker.

In another embodiment, the fusion protein provided herein comprises first and second IL-2 domains, first and second anti-PD-1 scFvs, an Fc domain comprising a first and second peptide chains, and optionally first and second peptide linker; wherein the C-terminus of the first anti-PD-1 scFv is connected to the N-terminus of the first peptide chain of the Fc domain directly or via a peptide linker, and the C-terminus of the first peptide chain of the Fc domain is connected to the N-terminus of the first IL-2 domain via the first peptide linker; and wherein the C-terminus of the second anti-PD-1 scFv is connected to the N-terminus of the second peptide chain of the Fc domain directly or via a peptide linker, and the C-terminus of the second peptide chain of the Fc domain is connected to the N-terminus of the second IL-2 domain via the second peptide linker.

In one embodiment, each anti-PD-1 scFv independently comprises:
(i) a light chain complementarity determining region 1 (CDRL1) of SEQ ID NO: 29,
   a light chain complementarity determining region 2 (CDRL2) of DAS,
   a light chain complementarity determining region 3 (CDRL3) of SEQ ID NO: 30,
   a heavy chain complementarity determining region 1 (CDRH1) of SEQ ID NO: 31,
   a heavy chain complementarity determining region 2 (CDRH2) of SEQ ID NO: 32, and a heavy chain complementarity determining region 3 (CDRH3) of SEQ ID NO: 33;
(ii) a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42;
(iii) a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51;
(iv) a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60;
(v) a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69;
(vi) a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78;
(vii) a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87;
(viii) a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96;
(ix) a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105; or
(x) a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

In one embodiment, each anti-PD-1 scFv comprises a CDRL1 of SEQ ID NO: 29, a CDRL2 of DAS, a CDRL3 of SEQ ID NO: 30, a CDRH1 of SEQ ID NO: 31, a CDRH2 of SEQ ID NO: 32, and a CDRH3 of SEQ ID NO: 33. In another embodiment, each anti-PD-1 scFv comprises a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42. In yet another embodiment, each anti-PD-1 scFv comprises a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51. In yet another embodiment, each anti-PD-1 scFv comprises a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60. In yet another embodiment, each anti-PD-1 scFv comprises a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69. In yet another embodiment, each anti-PD-1 scFv comprises a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78. In yet another embodiment, each anti-PD-1 scFv comprises a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87. In yet another embodiment, each anti-PD-1 scFv comprises a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96. In yet another embodiment, each anti-PD-1 scFv comprises a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105. In still another embodiment, each anti-PD-1 scFv comprises a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

In another embodiment, each anti-PD-1 scFv comprises:
(i) a light chain variable region of SEQ ID NO: 34 and a heavy chain variable region of SEQ ID NO: 35;
(ii) a light chain variable region of SEQ ID NO: 43 and a heavy chain variable region of SEQ ID NO: 44;
(iii) a light chain variable region of SEQ ID NO: 52 and a heavy chain variable region of SEQ ID NO: 53;
(iv) a light chain variable region of SEQ ID NO: 61 and a heavy chain variable region of SEQ ID NO: 62;
(v) a light chain variable region of SEQ ID NO: 70 and a heavy chain variable region of SEQ ID NO: 71;
(vi) a light chain variable region of SEQ ID NO: 79 and a heavy chain variable region of SEQ ID NO: 80;
(vii) a light chain variable region of SEQ ID NO: 88 and a heavy chain variable region of SEQ ID NO: 89;
(viii) a light chain variable region of SEQ ID NO: 97 and a heavy chain variable region of SEQ ID NO: 98;
(ix) a light chain variable region of SEQ ID NO: 106 and a heavy chain variable region of SEQ ID NO: 107; or
(xi) a light chain variable region of SEQ ID NO: 115 and a heavy chain variable region of SEQ ID NO: 116.

In one embodiment, each anti-PD-1 scFv comprises a light chain variable region of SEQ ID NO: 34 and a heavy chain variable region of SEQ ID NO: 35. In another embodiment, each anti-PD-1 scFv comprises a light chain variable region of SEQ ID NO: 43 and a heavy chain variable region of SEQ ID NO: 44. In yet another embodiment, each anti-PD-1 scFv comprises a light chain variable region of SEQ ID NO: 52 and a heavy chain variable region of SEQ ID NO: 53. In yet another embodiment, each anti-PD-1 scFv comprises a light chain variable region of SEQ ID NO: 61 and a heavy chain variable region of SEQ ID NO: 62. In yet another embodiment, each anti-PD-1 scFv comprises a light chain variable region of SEQ ID NO: 70 and a heavy chain variable region of SEQ ID NO: 71. In yet another embodiment, each anti-PD-1 scFv comprises a light chain variable region of SEQ ID NO: 79 and a heavy chain variable region of SEQ ID NO: 80. In yet another embodiment, each anti-PD-1 scFv comprises a light chain variable region of SEQ ID NO: 88 and a heavy chain variable region of SEQ ID NO: 89. In yet another embodiment, each anti-PD-1 scFv comprises a light chain variable region of SEQ ID NO: 97 and a heavy chain variable region of SEQ ID NO: 98. In yet another embodiment, each anti-PD-1 scFv comprises a light chain variable region of SEQ ID NO: 106 and a heavy chain variable region of SEQ ID NO: 107. In still another embodiment, each anti-PD-1 scFv comprises a light chain variable region of SEQ ID NO: 115 and a heavy chain variable region of SEQ ID NO: 116.

In one embodiment, each anti-PD-1 scFv comprising a light chain (V_{L}), a heavy chain (V_{H}), and optionally a peptide linker, wherein the C-terminal of the light chain is connected to the N-terminal of the heavy chain directly or via the peptide linker, or wherein the C-terminal of the heavy chain is connected to the N-terminal of the light chain directly or via the peptide linker. In another embodiment, each anti-PD-1 scFv comprising a light chain (V_{L}), a heavy chain (V_{H}), and optionally a peptide linker, wherein the C-terminal of the light chain is connected to the N-terminal of the heavy chain via the peptide linker, or wherein the C-terminal of the heavy chain is connected to the N-terminal of the light chain via the peptide linker. In yet another embodiment, each anti-PD-1 scFv comprising a light chain (V_{L}), a heavy chain (V_{H}), and optionally a peptide linker, wherein the C-terminal of the light chain is connected to the N-terminal of the heavy chain via the peptide linker. In still another embodiment, each anti-PD-1 scFv comprising a light chain (V_{L}), a heavy chain (V_{H}), and optionally a peptide linker, wherein the C-terminal of the heavy chain is connected to the N-terminal of the light chain via the peptide linker.

In one embodiment, the Fc domain is a hIgG1 Fc domain or a mutein thereof, or a fragment thereof. In another embodiment, the Fc domain is a hIgG1 Fc having an amino acid substitution of N297A. In yet another embodiment, the Fc domain is a hIgG2 Fc domain or a mutein thereof, or a fragment thereof. In still another embodiment, the Fc domain is a hIgG4 Fc domain or a mutein thereof, or a fragment thereof.

In one embodiment, the FC domain as a half-life-extension domain comprises an amino acid sequence of SEQ ID NO: 16, 17, 18, 19, 20, 21, 22, 23, 24, 25, 26, or 27. Thus, in one embodiment, the FC domain comprises an amino acid sequence of SEQ ID NO: 16. In yet another embodiment, the FC domain comprises an amino acid sequence of SEQ ID NO: 17. In yet another embodiment, the FC domain comprises an amino acid sequence of SEQ ID NO: 18. In yet another embodiment, the FC domain comprises an amino acid sequence of SEQ ID NO: 19. In yet another embodiment, the FC domain comprises an amino acid sequence of SEQ ID NO: 20. In yet another embodiment, the FC domain comprises an amino acid sequence of SEQ ID NO: 21. In yet another embodiment, the FC domain comprises an amino acid sequence of SEQ ID NO: 22. In yet another embodiment, the FC domain comprises an amino acid sequence of SEQ ID NO: 23. In yet another embodiment, the FC domain comprises an amino acid sequence of SEQ ID NO: 24. In yet another embodiment, the FC domain comprises an amino acid sequence of SEQ ID NO: 25. In yet another embodiment, the FC domain comprises an amino acid sequence of SEQ ID NO: 26. In still another embodiment, the FC domain comprises an amino acid sequence of SEQ ID NO: 27.

In one embodiment, the FC domain as a half-life-extension domain comprises a pair of peptide chains in a knobs-in-holes (KIH) configuration. Thus, in one embodiment, the FC domain comprises an amino acid sequence pair of SEQ ID NOs: 19 and 20, 21 and 22, 23 and 24, or 26 and 27 in a knobs-in-holes configuration. In another embodiment, the FC domain comprises an amino acid sequence pair of SEQ ID NOs: 19 and 20 in a knobs-in-holes configuration. In yet another embodiment, the FC domain comprises an amino acid sequence pair of SEQ ID NOs: 21 and 22 in a knobs-in-holes configuration. In yet another embodiment, the Fc domain comprises an amino acid sequence pair of SEQ ID NOs: 23 and 24 in a knobs-in-holes configuration. In still another embodiment, the FC domain comprises an amino acid sequence pair of SEQ ID NOs: 26 and 27 in a knobs-in-holes configuration.

In one embodiment, the PD-1 binding domain and the Fc domain in a fusion protein provided herein are parts of an intact anti-PD-1 antibody comprising two light chains and two heavy chains.

Thus, in one embodiment, provided herein is a fusion protein comprising an intact anti-PD-1 antibody comprising two light chains and two heavy chains, an IL-2 domain, and optionally a peptide linker.

In one embodiment, the fusion protein provided herein comprises an IL-2 domain, an intact anti-PD-1 antibody comprising first and second light chains and first and second heavy chains, and optionally a peptide linker; wherein the C-terminus of the first heavy chain of the intact anti-PD-1 antibody is connected to the N-terminus of the IL-2 domain directly or via the peptide linker.

In another embodiment, the fusion protein provided herein comprises an IL-2 domain, an intact anti-PD-1 antibody comprising first and second light chains and first and second heavy chains, and a peptide linker; wherein the C-terminus of the first heavy chain of the intact anti-PD-1 antibody is connected to the N-terminus of the IL-2 domain via the peptide linker.

In another embodiment, provided herein is a fusion protein comprising two IL-2 domains, and an intact anti-PD-1 antibody comprising two light chains and two heavy chains.

In one embodiment, the fusion protein provided herein comprises first and second IL-2 domains, an intact anti-PD-1 antibody comprising first and second light chains and first and second heavy chains, and optionally first and second peptide linkers; wherein the C-terminus of the first heavy chain of the intact anti-PD-1 antibody is connected to the N-terminus of the first IL-2 domain directly or via the first peptide linker, and the C-terminus of the second heavy chain of the intact anti-PD-1 antibody is connected to the N-terminus of the second IL-2 domain directly or via the second peptide linker.

In another embodiment, the fusion protein provided herein comprises first and second IL-2 domains, an intact anti-PD-1 antibody comprising first and second light chains and first and second heavy chains, and first and second peptide linkers; wherein the C-terminus of the first heavy chain of the intact anti-PD-1 antibody is connected to the N-terminus of the first IL-2 domain via the first peptide linker, and the C-terminus of the second heavy chain of the intact anti-PD-1 antibody is connected to the N-terminus of the second IL-2 domain via the second peptide linker.

In one embodiment, the intact anti-PD-1 antibody comprises:
(i) a light chain complementarity determining region 1 (CDRL1) of SEQ ID NO: 29,
   a light chain complementarity determining region 2 (CDRL2) of DAS,
   a light chain complementarity determining region 3 (CDRL3) of SEQ ID NO: 30,
   a heavy chain complementarity determining region 1 (CDRH1) of SEQ ID NO: 31,
   a heavy chain complementarity determining region 2 (CDRH2) of SEQ ID NO: 32, and a heavy chain complementarity determining region 3 (CDRH3) of SEQ ID NO: 33;
(ii) a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42;
(iii) a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51;
(iv) a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60;
(v) a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69;
(vi) a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78;
(vii) a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87;
(viii) a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96;
(ix) a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105; or
(x) a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

In one embodiment, the intact anti-PD-1 antibody comprises a CDRL1 of SEQ ID NO: 29, a CDRL2 of DAS, a CDRL3 of SEQ ID NO: 30, a CDRH1 of SEQ ID NO: 31, a CDRH2 of SEQ ID NO: 32, and a CDRH3 of SEQ ID NO: 33. In another embodiment, the intact anti-PD-1 antibody comprises a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42. In yet another embodiment, the intact anti-PD-1 antibody comprises a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51. In yet another embodiment, the intact anti-PD-1 antibody comprises a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60. In yet another embodiment, the intact anti-PD-1 antibody comprises a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69. In yet another embodiment, the intact anti-PD-1 antibody comprises a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78. In yet another embodiment, the intact anti-PD-1 antibody comprises a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87. In yet another embodiment, the intact anti-PD-1 antibody comprises a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96. In yet another embodiment, the intact anti-PD-1 antibody comprises a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105. In still another embodiment, the intact anti-PD-1 antibody comprises a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

In another embodiment, the intact anti-PD-1 antibody comprises:
(i) a light chain variable region of SEQ ID NO: 34 and a heavy chain variable region of SEQ ID NO: 35;
(ii) a light chain variable region of SEQ ID NO: 43 and a heavy chain variable region of SEQ ID NO: 44;
(iii) a light chain variable region of SEQ ID NO: 52 and a heavy chain variable region of SEQ ID NO: 53;
(iv) a light chain variable region of SEQ ID NO: 61 and a heavy chain variable region of SEQ ID NO: 62;
(v) a light chain variable region of SEQ ID NO: 70 and a heavy chain variable region of SEQ ID NO: 71;
(vi) a light chain variable region of SEQ ID NO: 79 and a heavy chain variable region of SEQ ID NO: 80;
(vii) a light chain variable region of SEQ ID NO: 88 and a heavy chain variable region of SEQ ID NO: 89;
(viii) a light chain variable region of SEQ ID NO: 97 and a heavy chain variable region of SEQ ID NO: 98;
(ix) a light chain variable region of SEQ ID NO: 106 and a heavy chain variable region of SEQ ID NO: 107; or
(x) a light chain variable region of SEQ ID NO: 115 and a heavy chain variable region of SEQ ID NO: 116.

In one embodiment, the intact anti-PD-1 antibody comprises a light chain variable region of SEQ ID NO: 34 and a heavy chain variable region of SEQ ID NO: 35. In another embodiment, the intact anti-PD-1 antibody comprises a light chain variable region of SEQ ID NO: 43 and a heavy chain variable region of SEQ ID NO: 44. In yet another embodiment, the intact anti-PD-1 antibody comprises a light chain variable region of SEQ ID NO: 52 and a heavy chain variable region of SEQ ID NO: 53. In yet another embodiment, the intact anti-PD-1 antibody comprises a light chain variable region of SEQ ID NO: 61 and a heavy chain variable region of SEQ ID NO: 62. In yet another embodiment, the intact anti-PD-1 antibody comprises a light chain variable region of SEQ ID NO: 70 and a heavy chain variable region of SEQ ID NO: 71. In yet another embodiment, the intact anti-PD-1 antibody comprises a light chain variable region of SEQ ID NO: 79 and a heavy chain variable region of SEQ ID NO: 80. In yet another embodiment, the intact anti-PD-1 antibody comprises a light chain variable region of SEQ ID NO: 88 and a heavy chain variable region of SEQ ID NO: 89. In yet another embodiment, the intact anti-PD-1 antibody comprises a light chain variable region of SEQ ID NO: 97 and a heavy chain variable region of SEQ ID NO: 98. In yet another embodiment, the intact anti-PD-1 antibody comprises a light chain variable region of SEQ ID NO: 106 and a heavy chain variable region of SEQ ID NO: 107. In still another embodiment, the intact anti-PD-1 antibody comprises a light chain variable region of SEQ ID NO: 115 and a heavy chain variable region of SEQ ID NO: 116.

In one embodiment, the intact anti-PD-1 antibody is an IgA1, IgA2, IgG1, IgG2, IgG3, or IgG4 antibody. In another embodiment, the intact anti-PD-1 antibody is an IgA1 or IgA2. In yet another embodiment, the intact anti-PD-1 antibody is an IgA1. In yet another embodiment, the anti-PD-1 intact antibody is an IgA2. In yet another embodiment, the intact anti-PD-1 antibody is an IgG1, IgG2, IgG3, or IgG4 antibody. In yet another embodiment, the intact anti-PD-1 antibody is an IgG1 antibody. In yet another embodiment, the intact anti-PD-1 antibody is an IgG1 antibody with an N297A mutation. In yet another embodiment, the intact anti-PD-1 antibody is an IgG2 antibody. In yet another embodiment, the intact anti-PD-1 antibody is an IgG3 antibody. In still another embodiment, the intact anti-PD-1 antibody is an IgG4 antibody.

In one embodiment, the light chain of the intact anti-PD-1 antibody is a kappa or lambda chain. In another embodiment, the light chain of the intact anti-PD-1 antibody is a kappa chain. In yet another embodiment, the light chain of the intact anti-PD-1 antibody is a lambda chain.

In one embodiment, the intact anti-PD-1 antibody is in a knobs-in-holes configuration. In another embodiment, the intact anti-PD-1 antibody is an IgG1 antibody comprising in a knobs-in-holes configuration. In yet another embodiment, the intact anti-PD-1 antibody is an IgG4 antibody in a knobs-in-holes configuration.

In yet another embodiment, the intact anti-PD-1 antibody comprises:
(i) a light chain of SEQ ID NO: 36 and a heavy chain of SEQ ID NO: 37;
(ii) a light chain of SEQ ID NO: 45 and a heavy chain of SEQ ID NO: 46;
(iii) a light chain of SEQ ID NO: 54 and a heavy chain of SEQ ID NO: 55;
(iv) a light chain of SEQ ID NO: 63 and a heavy chain of SEQ ID NO: 64;
(v) a light chain of SEQ ID NO: 72 and a heavy chain of SEQ ID NO: 73;
(vi) a light chain of SEQ ID NO: 81 and a heavy chain of SEQ ID NO: 82;
(vii) a light chain of SEQ ID NO: 90 and a heavy chain of SEQ ID NO: 91;
(viii) a light chain of SEQ ID NO: 99 and a heavy chain of SEQ ID NO: 100;
(ix) a light chain of SEQ ID NO: 108 and a heavy chain of SEQ ID NO: 109; or
(x) a light chain of SEQ ID NO: 117 and a heavy chain of SEQ ID NO: 118.

In one embodiment, the intact anti-PD-1 antibody comprises a light chain of SEQ ID NO: 36 and a heavy chain of SEQ ID NO: 37. In another embodiment, the intact anti-PD-1 antibody comprises a light chain of SEQ ID NO: 45 and a heavy chain of SEQ ID NO: 46. In yet another embodiment, the intact anti-PD-1 antibody comprises a light chain of SEQ ID NO: 54 and a heavy chain of SEQ ID NO: 55. In yet another embodiment, the intact anti-PD-1 antibody comprises a light chain of SEQ ID NO: 63 and a heavy chain of SEQ ID NO: 64. In yet another embodiment, the intact anti-PD-1 antibody comprises a light chain of SEQ ID NO: 72 and a heavy chain of SEQ ID NO: 73. In yet another embodiment, the intact anti-PD-1 antibody comprises a light chain of SEQ ID NO: 81 and a heavy chain of SEQ ID NO: 82. In yet another embodiment, the intact anti-PD-1 antibody comprises a light chain of SEQ ID NO: 90 and a heavy chain of SEQ ID NO: 91. In yet another embodiment, the intact anti-PD-1 antibody comprises a light chain of SEQ ID NO: 99 and a heavy chain of SEQ ID NO: 100. In yet another embodiment, the intact anti-PD-1 antibody comprises a light chain of SEQ ID NO: 108 and a heavy chain of SEQ ID NO: 109. In still another embodiment, the intact anti-PD-1 antibody comprises a light chain of SEQ ID NO: 117 and a heavy chain of SEQ ID NO: 118.

In one embodiment, each IL-2 domain in a fusion protein provided herein independently comprises an amino acid sequence of an IL-2 mutein provided herein. In another embodiment, each first IL-2 domain in a fusion protein provided herein independently comprises an amino acid sequence of an IL-2 mutein provided herein. In yet another embodiment, each second IL-2 domain in a fusion protein provided herein independently comprises an amino acid sequence of an IL-2 mutein provided herein.

In one embodiment, each peptide linker in a fusion protein provided herein independently comprises an amino acid sequence of GSG or one of SEQ ID NOs: 139 to 178. In another embodiment, each peptide linker in a fusion protein provided herein independently comprises an amino acid sequence of GSG or SEQ ID NO: 139, 140, or 141. In yet another embodiment, each peptide linker in a fusion protein provided herein independently comprises an amino acid sequence of SEQ ID NO: 142, 143, 144, or 145. In yet another embodiment, each peptide linker in a fusion protein provided herein independently comprises an amino acid sequence of SEQ ID NO: 146, 147, 148, or 149. In yet another embodiment, each peptide linker in a fusion protein provided herein independently comprises an amino acid sequence of SEQ ID NO: 150, 151, 152, or 153. In yet another embodiment, each peptide linker in a fusion protein provided herein independently comprises an amino acid sequence of SEQ ID NO: 154, 155, 156, or 157. In yet another embodiment, each peptide linker in a fusion protein provided herein independently comprises an amino acid sequence of SEQ ID NO: 158, 159, 160, or 161. In yet another embodiment, each peptide linker in a fusion protein provided herein independently comprises an amino acid sequence of SEQ ID NO: 162, 163, 164, or 165. In yet another embodiment, each peptide linker in a fusion protein provided herein independently comprises an amino acid sequence of SEQ ID NO: 166, 167, 168, or 169. In yet another embodiment, each peptide linker in a fusion protein provided herein independently comprises an amino acid sequence of SEQ ID NO: 170. In yet another embodiment, each peptide linker in a fusion protein provided herein independently comprises an amino acid sequence of SEQ ID NO: 171 or 172. In yet another embodiment, each peptide linker in a fusion protein provided herein independently comprises an amino acid sequence of SEQ ID NO: 173 or 174. In yet another embodiment, each peptide linker in a fusion protein provided herein independently comprises an amino acid sequence of SEQ ID NO: 175 or 176. In still another embodiment, each peptide linker in a fusion protein provided herein independently comprises an amino acid sequence of SEQ ID NO: 177 or 178.

In one embodiment, each first peptide linker in a fusion protein provided herein independently comprises an amino acid sequence of GSG or one of SEQ ID NOs: 139 to 178. In another embodiment, each first peptide linker in a fusion protein provided herein independently comprises an amino acid sequence of GSG or SEQ ID NO: 139, 140, or 141. In yet another embodiment, each first peptide linker in a fusion protein provided herein independently comprises an amino acid sequence of SEQ ID NO: 142, 143, 144, or 145. In yet another embodiment, each first peptide linker in a fusion protein provided herein independently comprises an amino acid sequence of SEQ ID NO: 146, 147, 148, or 149. In yet another embodiment, each first peptide linker in a fusion protein provided herein independently comprises an amino acid sequence of SEQ ID NO: 150, 151, 152, or 153. In yet another embodiment, each first peptide linker in a fusion protein provided herein independently comprises an amino acid sequence of SEQ ID NO: 154, 155, 156, or 157. In yet another embodiment, each first peptide linker in a fusion protein provided herein independently comprises an amino acid sequence of SEQ ID NO: 158, 159, 160, or 161. In yet another embodiment, each first peptide linker in a fusion protein provided herein independently comprises an amino acid sequence of SEQ ID NO: 162, 163, 164, or 165. In yet another embodiment, each first peptide linker in a fusion protein provided herein independently comprises an amino acid sequence of SEQ ID NO: 166, 167, 168, or 169. In yet another embodiment, each first peptide linker in a fusion protein provided herein independently comprises an amino acid sequence of SEQ ID NO: 170. In yet another embodiment, each first peptide linker in a fusion protein provided herein independently comprises an amino acid sequence of SEQ ID NO: 171 or 172. In yet another embodiment, each first peptide linker in a fusion protein provided herein independently comprises an amino acid sequence of SEQ ID NO: 173 or 174. In yet another embodiment, each first peptide linker in a fusion protein provided herein independently comprises an amino acid sequence of SEQ ID NO: 175 or 176. In still another embodiment, each first peptide linker in a fusion protein provided herein independently comprises an amino acid sequence of SEQ ID NO: 177 or 178.

In one embodiment, each second peptide linker in a fusion protein provided herein independently comprises an amino acid sequence of GSG or one of SEQ ID NOs: 139 to 178. In another embodiment, each second peptide linker in a fusion protein provided herein independently comprises an amino acid sequence of GSG or SEQ ID NO: 139, 140, or 141. In yet another embodiment, each second peptide linker in a fusion protein provided herein independently comprises an amino acid sequence of SEQ ID NO: 142, 143, 144, or 145. In yet another embodiment, each second peptide linker in a fusion protein provided herein independently comprises an amino acid sequence of SEQ ID NO: 146, 147, 148, or 149. In yet another embodiment, each second peptide linker in a fusion protein provided herein independently comprises an amino acid sequence of SEQ ID NO: 150, 151, 152, or 153. In yet another embodiment, each second peptide linker in a fusion protein provided herein independently comprises an amino acid sequence of SEQ ID NO: 154, 155, 156, or 157. In yet another embodiment, each second peptide linker in a fusion protein provided herein independently comprises an amino acid sequence of SEQ ID NO: 158, 159, 160, or 161. In yet another embodiment, each second peptide linker in a fusion protein provided herein independently comprises an amino acid sequence of SEQ ID NO: 162, 163, 164, or 165. In yet another embodiment, each second peptide linker in a fusion protein provided herein independently comprises an amino acid sequence of SEQ ID NO: 166, 167, 168, or 169. In yet another embodiment, each second peptide linker in a fusion protein provided herein independently comprises an amino acid sequence of SEQ ID NO: 170. In yet another embodiment, each second peptide linker in a fusion protein provided herein independently comprises an amino acid sequence of SEQ ID NO: 171 or 172. In yet another embodiment, each second peptide linker in a fusion protein provided herein independently comprises an amino acid sequence of SEQ ID NO: 173 or 174. In yet another embodiment, each second peptide linker in a fusion protein provided herein independently comprises an amino acid sequence of SEQ ID NO: 175 or 176. In still another embodiment, each second peptide linker in a fusion protein provided herein independently comprises an amino acid sequence of SEQ ID NO: 177 or 178.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 29, a CDRL2 of DAS, a CDRL3 of SEQ ID NO: 30, a CDRH1 of SEQ ID NO: 31, a CDRH2 of SEQ ID NO: 32, and a CDRH3 of SEQ ID NO: 33.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 34 and each heavy chain comprises the amino acid sequence of SEQ ID NO: 35.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 36 and each heavy chain comprises the amino acid sequence of SEQ ID NO: 37.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 43 and each heavy chain comprises the amino acid sequence of SEQ ID NO: 44.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 45 and each heavy chain comprises the amino acid sequence of SEQ ID NO: 46.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 52 and each heavy chain comprises the amino acid sequence of SEQ ID NO: 53.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 54 and each heavy chain comprises the amino acid sequence of SEQ ID NO: 55.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 61 and each heavy chain comprises the amino acid sequence of SEQ ID NO: 62.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 63 and each heavy chain comprises the amino acid sequence of SEQ ID NO: 64.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 70 and each heavy chain comprises the amino acid sequence of SEQ ID NO: 71.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 72 and each heavy chain comprises the amino acid sequence of SEQ ID NO: 73.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 79 and each heavy chain comprises the amino acid sequence of SEQ ID NO: 80.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 81 and each heavy chain comprises the amino acid sequence of SEQ ID NO: 82.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 88 and each heavy chain comprises the amino acid sequence of SEQ ID NO: 89.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 90 and each heavy chain comprises the amino acid sequence of SEQ ID NO: 91.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 97 and each heavy chain comprises the amino acid sequence of SEQ ID NO: 98.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 99 and each heavy chain comprises the amino acid sequence of SEQ ID NO: 100.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 106 and each heavy chain comprises the amino acid sequence of SEQ ID NO: 107.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 108 and each heavy chain comprises the amino acid sequence of SEQ ID NO: 109.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and the anti-PD-1 antibody comprises a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 115 and each heavy chain comprises the amino acid sequence of SEQ ID NO: 116.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of any one of SEQ ID NOs: 262 to 287; and each light chain comprises the amino acid sequence of SEQ ID NO: 117 and each heavy chain comprises the amino acid sequence of SEQ ID NO: 118.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 29, a CDRL2 of DAS, a CDRL3 of SEQ ID NO: 30, a CDRH1 of SEQ ID NO: 31, a CDRH2 of SEQ ID NO: 32, and a CDRH3 of SEQ ID NO: 33.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 34 and each heavy chain comprises the amino acid sequence of SEQ ID NO: 35.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 36 and each heavy chain comprises the amino acid sequence of SEQ ID NO: 37.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 43 and each heavy chain comprises the amino acid sequence of SEQ ID NO: 44.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 45 and each heavy chain comprises the amino acid sequence of SEQ ID NO: 46.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 52 and each heavy chain comprises the amino acid sequence of SEQ ID NO: 53.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 54 and each heavy chain comprises the amino acid sequence of SEQ ID NO: 55.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 61 and each heavy chain comprises the amino acid sequence of SEQ ID NO: 62.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 63 and each heavy chain comprises the amino acid sequence of SEQ ID NO: 64.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 70 and each heavy chain comprises the amino acid sequence of SEQ ID NO: 71.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 72 and each heavy chain comprises the amino acid sequence of SEQ ID NO: 73.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 79 and each heavy chain comprises the amino acid sequence of SEQ ID NO: 80.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 81 and each heavy chain comprises the amino acid sequence of SEQ ID NO: 82.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 88 and each heavy chain comprises the amino acid sequence of SEQ ID NO: 89.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 90 and each heavy chain comprises the amino acid sequence of SEQ ID NO: 91.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 97 and each heavy chain comprises the amino acid sequence of SEQ ID NO: 98.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 99 and each heavy chain comprises the amino acid sequence of SEQ ID NO: 100.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 106 and each heavy chain comprises the amino acid sequence of SEQ ID NO: 107.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 108 and each heavy chain comprises the amino acid sequence of SEQ ID NO: 109.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and the anti-PD-1 antibody comprises a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 115 and each heavy chain comprises the amino acid sequence of SEQ ID NO: 116.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of any one of SEQ ID NOs: 262 to 287; and each light chain comprises the amino acid sequence of SEQ ID NO: 117 and each heavy chain comprises the amino acid sequence of SEQ ID NO: 118.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 29, a CDRL2 of DAS, a CDRL3 of SEQ ID NO: 30, a CDRH1 of SEQ ID NO: 31, a CDRH2 of SEQ ID NO: 32, and a CDRH3 of SEQ ID NO: 33.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 34 and each heavy chain comprises the amino acid sequence of SEQ ID NO: 35.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 36 and each heavy chain comprises the amino acid sequence of SEQ ID NO: 37.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 43 and each heavy chain comprises the amino acid sequence of SEQ ID NO: 44.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 45 and each heavy chain comprises the amino acid sequence of SEQ ID NO: 46.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 52 and each heavy chain comprises the amino acid sequence of SEQ ID NO: 53.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 54 and each heavy chain comprises the amino acid sequence of SEQ ID NO: 55.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 61 and each heavy chain comprises the amino acid sequence of SEQ ID NO: 62.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 63 and each heavy chain comprises the amino acid sequence of SEQ ID NO: 64.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 70 and each heavy chain comprises the amino acid sequence of SEQ ID NO: 71.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 72 and each heavy chain comprises the amino acid sequence of SEQ ID NO: 73.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 79 and each heavy chain comprises the amino acid sequence of SEQ ID NO: 80.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 81 and each heavy chain comprises the amino acid sequence of SEQ ID NO: 82.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 88 and each heavy chain comprises the amino acid sequence of SEQ ID NO: 89.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 90 and each heavy chain comprises the amino acid sequence of SEQ ID NO: 91.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 97 and each heavy chain comprises the amino acid sequence of SEQ ID NO: 98.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 99 and each heavy chain comprises the amino acid sequence of SEQ ID NO: 100.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 106 and each heavy chain comprises the amino acid sequence of SEQ ID NO: 107.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 108 and each heavy chain comprises the amino acid sequence of SEQ ID NO: 109.

In one embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and the anti-PD-1 antibody comprises a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

In another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 115 and each heavy chain comprises the amino acid sequence of SEQ ID NO: 116.

In yet another embodiment, provided herein is a fusion protein comprising an IL-2 domain, an intact anti-PD-1 antibody comprising two light chains and first and second heavy chains, and a peptide linker, wherein the C-terminus of the first heavy chain of the anti-PD1 antibody is connected to the N-terminus of the IL-2 domain via the peptide linker; and wherein the IL-2 domain comprises the amino acid sequence of SEQ ID NO: 284, 285, or 301; and each light chain comprises the amino acid sequence of SEQ ID NO: 117 and each heavy chain comprises the amino acid sequence of SEQ ID NO: 118.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 29, a CDRL2 of DAS, a CDRL3 of SEQ ID NO: 30, a CDRH1 of SEQ ID NO: 31, a CDRH2 of SEQ ID NO: 32, and a CDRH3 of SEQ ID NO: 33.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 34 and each heavy chain comprises the amino acid sequence of SEQ ID NO: 35.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 36 and each heavy chain comprises the amino acid sequence of SEQ ID NO: 37.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 43 and each heavy chain comprises the amino acid sequence of SEQ ID NO: 44.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 45 and each heavy chain comprises the amino acid sequence of SEQ ID NO: 46.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 52 and each heavy chain comprises the amino acid sequence of SEQ ID NO: 53.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 54 and each heavy chain comprises the amino acid sequence of SEQ ID NO: 55.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 61 and each heavy chain comprises the amino acid sequence of SEQ ID NO: 62.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 63 and each heavy chain comprises the amino acid sequence of SEQ ID NO: 64.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 70 and each heavy chain comprises the amino acid sequence of SEQ ID NO: 71.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 72 and each heavy chain comprises the amino acid sequence of SEQ ID NO: 73.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 79 and each heavy chain comprises the amino acid sequence of SEQ ID NO: 80.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 81 and each heavy chain comprises the amino acid sequence of SEQ ID NO: 82.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 88 and each heavy chain comprises the amino acid sequence of SEQ ID NO: 89.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 90 and each heavy chain comprises the amino acid sequence of SEQ ID NO: 91.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 97 and each heavy chain comprises the amino acid sequence of SEQ ID NO: 98.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 99 and each heavy chain comprises the amino acid sequence of SEQ ID NO: 100.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 106 and each heavy chain comprises the amino acid sequence of SEQ ID NO: 107.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 108 and each heavy chain comprises the amino acid sequence of SEQ ID NO: 109.

In one embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and the anti-PD-1 antibody comprises a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

In another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 115 and each heavy chain comprises the amino acid sequence of SEQ ID NO: 116.

In yet another embodiment, provided herein is a fusion protein comprising first and second IL-2 domains, an intact anti-PD-1 antibody comprising two light chains and first and second heavy chains, and first and second peptide linkers, wherein the C-terminus of the first heavy chain of the anti-PD1 antibody is connected to the N-terminus of the first IL-2 domain via the first peptide linker, and the C-terminus of the second heavy chain of the anti-PD1 antibody is connected to the N-terminus of the second IL-2 domain via the second peptide linker; and wherein the IL-2 domain comprises the amino acid sequence of SEQ ID NO: 284, 285, or 301; and each light chain comprises the amino acid sequence of SEQ ID NO: 117 and each heavy chain comprises the amino acid sequence of SEQ ID NO: 118.

In certain embodiments, the PD-1 binding domain does not block the binding interaction between a PD-1 and a PD-L1. In certain embodiments, the PD-1 binding domain blocks the binding interaction between a PD-1 and a PD-L1.

In certain embodiments, the anti-PD-1 antibody is a non-blocking antibody, that is, an antibody does not block the binding interaction between a PD-1 and a PD-L1. In certain embodiments, the anti-PD-1 antibody is a blocking antibody, that is, an antibody blocks the binding interaction between a PD-1 and a PD-L1.

In one embodiment, provided herein is:
PD-L1-IL-2 fusion protein A1 comprising amino acid sequences of SEQ ID NOs: 36, 292, and 300;
PD-L1-IL-2 fusion protein A2 comprising amino acid sequences of SEQ ID NOs: 36, 293, and 300;
PD-L1-IL-2 fusion protein A3 comprising amino acid sequences of SEQ ID NOs: 36, 294, and 300;
PD-L1-IL-2 fusion protein A4 comprising amino acid sequences of SEQ ID NOs: 36, 295, and 300;
PD-L1-IL-2 fusion protein A5 comprising amino acid sequences of SEQ ID NOs: 36, 296, and 300;
PD-L1-IL-2 fusion protein A6 comprising amino acid sequences of SEQ ID NOs: 36, 297, and 300; or
PD-L1-IL-2 fusion protein A7 comprising amino acid sequences of SEQ ID NOs: 36, 298, and 300.

In another embodiment, provided herein is PD-L1-IL-2 fusion protein A8 comprising amino acid sequences of SEQ ID NOs: 36, 299, and 300.

### Pharmaceutical Compositions

In one embodiment, provided herein is a pharmaceutical composition comprising an IL-2 mutein or fusion protein provided herein, and a pharmaceutically acceptable excipient.

In one embodiment, the pharmaceutical composition is formulated as single dosage form.

In one embodiment, the pharmaceutical composition provided herein is a solid formulation. In another embodiment, the pharmaceutical composition provided herein is a lyophilized solid formulation. In yet another embodiment, the pharmaceutical composition provided herein is a solution. In still another embodiment, the pharmaceutical composition provided herein is an aqueous solution.

In one embodiment, the pharmaceutical composition provided herein is formulated in a dosage form for parenteral administration. In another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for intratumoral administration.

### Methods of Use

In one embodiment, provided herein is a method of treating, preventing, or ameliorating one or more symptoms of a disorder, disease, or condition mediated by a PD-1 in a subject, comprising administering to the subject in need thereof a therapeutically effective amount of an IL-2 mutein or fusion protein provided herein.

In certain embodiments, the disorder, disease, or condition mediated by a PD-1 is a proliferative disease.

In another embodiment, provided herein is a method of treating, preventing, or ameliorating one or more symptoms of a disorder, disease, or condition mediated by an IL-2 in a subject, comprising administering to the subject in need thereof a therapeutically effective amount of an IL-2 mutein or fusion protein provided herein.

In certain embodiments, the disorder, disease, or condition mediated by an IL-2 is a proliferative disease.

In yet another embodiment, provided herein is a method for treating, preventing, or ameliorating a proliferative disease in a subject, comprising administering to the subject in need thereof a therapeutically effective amount of an IL-2 mutein or fusion protein provided herein.

In one embodiment, the proliferative disease is cancer. In another embodiment, the proliferative disease is colon cancer or colorectal cancer.

In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is resectable. In certain embodiments, the cancer is unresectable. In certain embodiments, the cancer is metastatic.

In certain embodiments, the cancer is drug-resistant. In certain embodiments, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the subject is a mammal. In certain embodiments, the subject is a human.

In another embodiment, provided herein is a method of inhibiting the growth of a cell, comprising contacting the cell with an effective amount of an IL-2 mutein or fusion protein provided herein. In certain embodiments, the cell is a cancerous cell. In certain embodiments, the cell is a human cancerous cell. In certain embodiments, the cell is a metastatic cancerous cell.

In certain embodiments, the therapeutically effective amount is ranging from about 0.001 mg per kg subject body weight every month (mg/kg per month) to 100 mg per kg subject body weight per day (mg/kg per day), from about 0.01 mg/kg per month to about 75 mg/kg per day, from about 0.1 mg/kg per month to about 50 mg/kg per day, from about 0.5 mg/kg per month to about 25 mg/kg per day, or from about 1 mg/kg per month to about 20 mg/kg per day, which can be administered in single or multiple doses. Within this range, the dosage can be ranging from about 0.005 mg/kg per month to about 0.05 mg/kg per day, from about 0.05 mg/kg per month to about 0.5 mg/kg per day, from about 0.5 mg/kg per month to about 5.0 mg/kg per day, from about 1 mg/kg per month to about 15 mg/kg per day, from about 1 mg/kg per month to about 20 mg/kg per day, or from about 1 mg/kg per month to about 50 mg/kg per day.

The disclosure will be further understood by the following non-limiting examples.

### EXAMPLES

### Example 1

### Cloning, Expression, and Purification of Fusion Proteins

The amino acid sequence of human IL-2 (hIL-2) was obtained from UNIPROT (hIL-2: P60568, 21-153 aa). A mutant hIL-2 was generated by introducing a mutation to attenuating CD25 and CD122/CD132 bindings. The amino acid sequences of a human anti-PD-1 antibody (VH & VL) were obtained from the Therapeutic Antibody Database (TABS). The deoxyoligonucleotide (DNA) sequences encoding the mutant hIL-2 and human anti-PD-1 antibody were codon optimized for CHO cell expression.

The DNA sequences encoding (i) the hIL-2 mutein, (ii) the human anti-PD-1 antibody or a fragment thereof, and (iii) a peptide linker were seamlessly assembled together by homology assembly cloning with commercially available kits. The oligonucleotides of each fusion protein were inserted into a UCOE® expression vector CET1019-AS-Puro for CHO cell expression.

Each fusion protein produced in the CHO cells was purified by a two-step purification process: protein A affinity chromatography using protein A (e.g., AMSPHERE™ A3 or MABSELECT™ SURE™) resin, and ion exchange chromatography (e.g., CAPTO™ Q IMPRES or CAPTO™ S IMPACT) or hydrophobic interaction chromatography (e.g., Phenyl HP).

### Example 2

### Binding Studies of IL-2 Muteins to CD122 and CD122/CD132

OCTET® RED96 was used to characterize the interactions of wild-type hL-2 and hhL-2 muteins with CD25, CD122, or CD122/CD132 heterodimer. AVT-TAG CD122/CD132 heterodimer was purchased from ACROBIO. The protein samples were diluted in the OCTET® kinetic buffer. Briefly, hIL-2 receptors were loaded onto a biosensor. The biosensor was then dipped into a solution containing a wild-type hL-2 and IL-2 muteins at 10 to 2,000 nM at 24° C. The association and dissociation sensorgraph was fitted by global or local fitting using the OCTET® Data Analysis HT software. The results are summarized in Table 1. All the hIL-2 muteins tested were found to have abolished or significantly reduced CD25 binding.

TABLE 1
Interactions of Wild-type hIL-2 and hIL-2 Muteins
with CD122 or CD122/CD132

| | | K_{d} (nM) | |
|---|---|---|---|
| Fusion Protein | SEQ ID NO: | CD122 | CD122/CD132 |
| WT IL-2 | 179 | | 1.9 |
| hIL-2 Mutein | 200 | | 1 |
| (T3A, R38E, F42A, | | | |
| K76E, C125S) | | | |
| IL-2-IL-15 Chimera 2 | 221 | 104 | 11 |
| (T3A, C125S) | | | |
| IL-2-IL-15 Chimera 29 | 248 | | 3.5 |
| (I92A, C125S) | | | |
| IL-2-IL-15 Chimera 42 | 261 | | 53 |
| (E15K, N88A, C125S) | | | |
| IL-2-IL-15 Chimera 44 | 263 | | 12 |
| (T3A, L18C) | | | |
| IL-2-IL-15 Chimera 46 | 265 | | 219 |
| (T3A, C125S, Q126E) | | | |
| IL-2-IL-15 Chimera 54 | 273 | 650 | 30 |
| (T3A, C125S, Q126T) | | | |
| IL-2-IL-15 Chimera 56 | 275 | 808 | 12 |
| (T3A, C125S, S130R) | | | |
| IL-2-IL-15 Chimera 58 | 277 | 188 | 229 |
| (T3A, L18C, Q126E) | | | |
| IL-2-IL-15 Chimera 64 | 283 | | 382 |
| (T3A, L18C, Q126S) | | | |
| IL-2-IL-15 Chimera 65 | 285 | | 242 |
| (T3A, I92A, C125S, | | | |
| Q126E) | | | |
| IL-2-IL-15 Chimera 68 | 287 | 466 | 32 |
| (T3A, C125S, Q126T, | | | |
| S130R) | | | |
| hIL-2 Mutein | 288 | 550 | 6.8 |
| (T3A, F42A, Y45A, | | | |
| L72G, C125S) | | | |
| IL-2-IL-15 Chimera 69 | 301 | | 322 |
| (T3A, L18S, C125S, | | | |
| Q126E) | | | |

### Example 3

### Antitumor Activity of Anti-PD-1/IL-2 Fusion Protein in a Xenograft Mouse Model

MC38 cells are cultured and maintained in DMEM media supplemented with 10% fetal bovine serum, GLUTAMAX™, non-essential amino acids (NEAA), sodium pyruvate, and penicillin/streptomycin. The cells are trypsinized, washed with the media, and counted. The cells are diluted with PBS and 5×10⁵ cells in PBS (50 μL) are injected subcutaneously into anesthetized C57BL/6 mice using an 18-gauge needle. A stock solution of an anti-PD-1/IL-2 fusion protein is diluted in PBS on the day of dosing and the mice are dosed intraperitoneally with PBS (control) or the anti-PD-1/IL-2 fusion protein in PBS twice a week for two weeks. Tumor sizes (length (L) and width (W)) are measured twice per week using a digital caliper, and the tumor volume is calculated (L×W×W)/2.

### Example 4. Antitumor Activity of Anti-PD-1/IL-2 Fusion Protein in a Xenograft Model Using PD-1 Knock-In Mice

MC38 cells or B16F10 cells are cultured and maintained in DMEM media supplemented with 10% fetal bovine serum, GLUTAMAX™, non-essential amino acids (NEAA), sodium pyruvate, and penicillin/streptomycin. The cells are trypsinized, washed with the media, and counted. The cells are diluted with PBS and 5×10⁵ cells in PBS (50 μL) are injected subcutaneously into anesthetized C57BL/6 mice with human PD-1 knock-in using an 18-gauge needle. A stock solution of an anti-PD-1/IL-2 fusion protein is diluted in PBS on the day of dosing and the mice are dosed intraperitoneally with PBS (control) or the anti-PD-1/IL-2 fusion protein in PBS twice a week for two weeks. Tumor sizes (length (L) and width (W)) are measured twice per week using a digital caliper, and the tumor volume is calculated (L×W×W)/2.

### Example 5

### Antitumor Activity of Anti-PD-1/IL-2 Fusion Protein in a Xenograft Mouse Model

CT26 mouse cells are cultured and maintained in RPMI media supplemented with 10% fetal bovine serum, GLUTAMAX™, and penicillin/streptomycin. The cells are trypsinized, washed with media, and counted. The cells are diluted with PBS and 1×10⁶ cells in PBS (100 μL) are injected subcutaneously into anesthetized BALB/c mice using an 18-gauge needle. A stock solution of an anti-PD-1/IL-2 fusion protein is diluted in PBS on the day of dosing and the mice are dosed intraperitoneally with PBS (control) or the anti-PD-1/IL-2 fusion protein in PBS twice a week for two weeks. Tumor sizes (length (L) and width (W)) are measured twice per week using a digital caliper, and the tumor volume is calculated (L×W×W)/2.

### Example 6

### Antitumor Activity of Anti-PD-1/IL-2 Fusion Protein in a Xenograft Mouse Model

HT-29 cells are cultured and maintained in McCoys 5a media supplemented with 10% fetal bovine serum and penicillin/streptomycin. The cells are trypsinized, washed with media, counted, and washed with PBS. The cell suspension (1×10⁶ cells in PBS (100 μL)) is injected subcutaneously into anesthetized NCG mice using a 27-gauge needle. After 6 days, human PBMCs (1×10⁷ cells in PBS (100 μL)) are injected into the tail vein of each mouse. A stock solution of an anti-PD-1/IL-2 fusion protein is diluted in PBS on the day of dosing and the mice are dosed intraperitoneally with PBS (control) or the anti-PD-1/IL-2 fusion protein in PBS twice a week for two weeks. Tumor sizes (length (L) and width (W)) are measured twice per week using a digital caliper, and the tumor volume is calculated (L×W×W)/2.

### Example 7

### Effect of Anti-PD-1/IL-2 Fusion Proteins on STAT5 Signaling

Anti-PD-1/IL-2 fusion proteins are evaluated for their effect on pSTAT5 signaling via its IL-2 domain in CD3 T-cells. Briefly, CD3 T-cells (100,000) are treated with an anti-PD-1/IL-2 fusion protein for 30 min at 37° C. and 5% C02 in Hanks balanced salt solution containing 10 mM HEPES. Phospho-STAT5 is measured using a phosphor-STAT5 (Tyr694) HTRF assay. The signal ratio at 665 nm/620 nm is multiplied by 1,000 and the data is analyzed with global fitting to determine EC₅₀ values.

Sequences described herein are provided in the sequence table below.

SEQUENCE TABLE

| SEQ ID NO: | Description | Amino Acid Sequence |
|---|---|---|
| 1 | Anti-HSA-1 CDR1 | GSTWSINT |
| 2 | Anti-HSA-1 CDR2 | ISSGGST |
| 3 | Anti-HSA-1 CDR3 | YAQSTWYPPS |
| 4 | Anti-HSA-1 FR1 | EVQLVESGGGLVQPGGSLRLSCAAS |
| 5 | Anti-HSA-1 FR2 | LAWYRQAPGKQRDLVAR |
| 6 | Anti-HSA-1 FR3 | YYADSVKGRFTISRDNSKNTLYLQMNSLRAEDTAV |
| | | YYC |
| 7 | Anti-HSA-1 FR4 | WGQGTLVTVSS |
| 8 | Anti-HSA-1 | EVQLVESGGGLVQPGGSLRLSCAASGSTWSINTLA |
| | | WYRQAPGKQRDLVARISSGGSTYYADSVKGRFTIS |
| | | RDNSKNTLYLQMNSLRAEDTAVYYCYAQSTWYPP |
| | | SWGQGTLVTVSS |
| 9 | Anti-HSA-2 CDR1 | GFAFRGFG |
| 10 | Anti-HSA-2 CDR2 | INNGGSDT |
| 11 | Anti-HSA-2 CDR3 | AIGGPGASP |
| 12 | Anti-HSA-2 FR1 | QVQLVESGGGVVQPGGSLRLSCAAS |
| 13 | Anti-HSA-2 FR2 | MSWVRQAPGKGLEWVSS |
| 14 | Anti-HSA-2 FR4 | SGQGTQVTVSS |
| 15 | Anti-HSA-2 | QVQLVESGGGVVQPGGSLRLSCAASGFAFRGFGM |
| | | SWVRQAPGKGLEWVSSINNGGSDTYYADSVKGRF |
| | | TISRDNSKNTLYLQMNSLRAEDTAVYYCAIGGPG |
| | | ASPSGQGTQVTVSS |
| 16 | hIgG1 C_{H} | ASTKGPSVFPLAPSSKSTSGGTAALGCLVKDYFPEP |
| | | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPE |
| | | VTCVVVDVSHEDPEVKFNWYVDGVEVHNAKTKPRE |
| | | EQYNSTYRVVSVLTVLHQDWLNGKEYKCKVSNKA |
| | | LPAPIEKTISKAKGQPREPQVYTLPPSREEMTKNQ |
| | | VSLTCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 17 | hIgG1 C_{H} Mutein | ASTKGPSVFPLAPSSKSTSGGTAALGCLVKDYFPEP |
| | (N297A) | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPE |
| | | VTCVVVDVSHEDPEVKFNWYVDGVEVHNAKTKPRE |
| | | EQYASTYRVVSVLTVLHQDWLNGKEYKCKVSNKA |
| | | LPAPIEKTISKAKGQPREPQVYTLPPSREEMTKNQ |
| | | VSLTCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 18 | hIgG1 C_{H} Mutein | ASTKGPSVFPLAPSSKSTSGGTAALGCLVKDYFPEP |
| | (LALA, N297A) | VTVSWNSGALTSGVHTFPAVLQSSGLYSLSSVVTV |
| | | PSSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTKPRE |
| | | EQYASTYRVVSVLTVLHQDWLNGKEYKCKVSNKA |
| | | LPAPIEKTISKAKGQPREPQVYTLPPSREEMTKNQ |
| | | VSLTCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 19 | hIgG1 C_{H} Mutein | ASTKGPSVFPLAPSSKSTSGGTAALGCLVKDYFPEP |
| | (S354C, T366W) | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPE |
| | | VTCVVVDVSHEDPEVKFNWYVDGVEVHNAKTKPRE |
| | | EQYNSTYRVVSVLTVLHQDWLNGKEYKCKVSNKA |
| | | LPAPIEKTISKAKGQPREPQVYTLPPCREEMTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 20 | hIgG1 C_{H} Mutein | ASTKGPSVFPLAPSSKSTSGGTAALGCLVKDYFPEP |
| | (Y349C, T366S, L368A, | VTVSWNSGALTSGVHTFPAVLQSSGLYSLSSVVTV |
| | Y407V) | PSSSLGTQTYICNVNHKPSNTKVDKKVEPKSCDKT |
| | | HTCPPCPAPELLGGPSVFLFPPKPKDTLMISRTPE |
| | | VTCVVVDVSHEDPEVKFNWYVDGVEVHNAKTKPRE |
| | | EQYNSTYRVVSVLTVLHQDWLNGKEYKCKVSNKA |
| | | LPAPIEKTISKAKGQPREPQVCTLPPSREEMTKNQ |
| | | VSLSCAVKGFYPSDIAVEWESNGQPENNYKTTPPV |
| | | LDSDGSFFLVSKLTVDKSRWQQGNVFSCSVMHEA |
| | | LHNHYTQKSLSLSPGK |
| 21 | hIgG1 C_{H} Mutein | ASTKGPSVFPLAPSSKSTSGGTAALGCLVKDYFPEP |
| | (S354C, T366W, N297A) | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPE |
| | | VTCVVVDVSHEDPEVKFNWYVDGVEVHNAKTKPRE |
| | | EQYASTYRVVSVLTVLHQDWLNGKEYKCKVSNKA |
| | | LPAPIEKTISKAKGQPREPQVYTLPPCREEMTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 22 | hIgG1 C_{H} Mutein | ASTKGPSVFPLAPSSKSTSGGTAALGCLVKDYFPEP |
| | (Y349C, T366S, L368A, | VTVSWNSGALTSGVHTFPAVLQSSGLYSLSSVVTV |
| | Y407V, N297A) | PSSSLGTQTYICNVNHKPSNTKVDKKVEPKSCDKT |
| | | HTCPPCPAPELLGGPSVFLFPPKPKDTLMISRTPE |
| | | VTCVVVDVSHEDPEVKFNWYVDGVEVHNAKTKPRE |
| | | EQYASTYRVVSVLTVLHQDWLNGKEYKCKVSNKA |
| | | LPAPIEKTISKAKGQPREPQVCTLPPSREEMTKNQ |
| | | VSLSCAVKGFYPSDIAVEWESNGQPENNYKTTPPV |
| | | LDSDGSFFLVSKLTVDKSRWQQGNVFSCSVMHEA |
| | | LHNHYTQKSLSLSPGK |
| 23 | hIgG1 C_{H} Mutein | ASTKGPSVFPLAPSSKSTSGGTAALGCLVKDYFPEP |
| | (S354C, T366W, LALA, | VTVSWNSGALTSGVHTFPAVLQSSGLYSLSSVVTV |
| | N297A) | PSSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTKPRE |
| | | EQYASTYRVVSVLTVLHQDWLNGKEYKCKVSNKA |
| | | LPAPIEKTISKAKGQPREPQVYTLPPCREEMTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 24 | hIgG1 C_{H} Mutein | ASTKGPSVFPLAPSSKSTSGGTAALGCLVKDYFPEP |
| | (Y349C, T366S, L368A, | VTVSWNSGALTSGVHTFPAVLQSSGLYSLSSVVTV |
| | Y407V, LALA, N297A) | PSSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTKPRE |
| | | EQYASTYRVVSVLTVLHQDWLNGKEYKCKVSNKA |
| | | LPAPIEKTISKAKGQPREPQVCTLPPSREEMTKNQ |
| | | VSLSCAVKGFYPSDIAVEWESNGQPENNYKTTPPV |
| | | LDSDGSFFLVSKLTVDKSRWQQGNVFSCSVMHEA |
| | | LHNHYTQKSLSLSPGK |
| 25 | hIgG4 C_{H} | ASTKGPSVFPLAPCSRSTSESTAALGCLVKDYFPEP |
| | | VTVSWNSGALTSGVHTFPAVLQSSGLYSLSSVVTV |
| | | PSSSLGTKTYTCNVDHKPSNTKVDKRVESKYGPPC |
| | | PPCPAPEFLGGPSVFLFPPKPKDTLMISRTPEVTC |
| | | VVVDVSQEDPEVQFNWYVDGVEVHNAKTKPREEQFN |
| | | STYRVVSVLTVLHQDWLNGKEYKCKVSNKGLPSSI |
| | | EKTISKAKGQPREPQVYTLPPSQEEMTKNQVSLTCL |
| | | VKGFYPSDIAVEWESNGQPENNYKTTPPVLDSDGS |
| | | FFLYSRLTVDKSRWQEGNVFSCSVMHEALHNHYT |
| | | QKSLSLSLGK |
| 26 | hIgG4 C_{H} Mutein | ASTKGPSVFPLAPCSRSTSESTAALGCLVKDYFPEP |
| | (T366W) | VTVSWNSGALTSGVHTFPAVLQSSGLYSLSSVVTV |
| | | PSSSLGTKTYTCNVDHKPSNTKVDKRVESKYGPPC |
| | | PPCPAPEFLGGPSVFLFPPKPKDTLMISRTPEVTCV |
| | | VVDVSQEDPEVQFNWYVDGVEVHNAKTKPREEQFN |
| | | STYRVVSVLTVLHQDWLNGKEYKCKVSNKGLPSSI |
| | | EKTISKAKGQPREPQVYTLPPSQEEMTKNQVSLWC |
| | | LVKGFYPSDIAVEWESNGQPENNYKTTPPVLDSDG |
| | | SFFLYSRLTVDKSRWQEGNVFSCSVMHEALHNHY |
| | | TQKSLSLSLGK |
| 27 | hIgG4 C_{H} Mutein | ASTKGPSVFPLAPCSRSTSESTAALGCLVKDYFPEP |
| | (T366S, T368A) | VTVSWNSGALTSGVHTFPAVLQSSGLYSLSSVVTV |
| | | PSSSLGTKTYTCNVDHKPSNTKVDKRVESKYGPPC |
| | | PPCPAPEFLGGPSVFLFPPKPKDTLMISRTPEVTCV |
| | | VVDVSQEDPEVQFNWYVDGVEVHNAKTKPREEQFN |
| | | STYRVVSVLTVLHQDWLNGKEYKCKVSNKGLPSSI |
| | | EKTISKAKGQPREPQVYTLPPSQEEMTKNQVSLSC |
| | | AVKGFYPSDIAVEWESNGQPENNYKTTPPVLDSDG |
| | | SFFLVSRLTVDKSRWQEGNVFSCSVMHEALHNHY |
| | | TQKSLSLSLGK |
| 28 | hKappa C_{L} | RTVAAPSVFIFPPSDEQLKSGTASVVCLLNNFYPR |
| | | EAKVQWKVDNALQSGNSQESVTEQDSKDSTYSLSS |
| | | TLTLSKADYEKHKVYACEVTHQGLSSPVTKSFNRG |
| | | EC |
| 29 | Anti-hPD-1-A CDRL1 | QSVSSY |
| | (IMGT) | |
| | Anti-hPD-1-A CDRL2 | DAS |
| | (IMGT) | |
| 30 | Anti-hPD-1-A CDRL3 | QQSSNWPRT |
| | (IMGT) | |
| 31 | Anti-hPD-1-A CDRH1 | GITFSNSG |
| | (IMGT) | |
| 32 | Anti-hPD-1-A CDRH2 | IWYDGSKR |
| | (IMGT) | |
| 33 | Anti-hPD-1-A CDRH3 | ATNDDY |
| | (IMGT) | |
| 34 | Anti-hPD-1-A Light Chain | EIVLTQSPATLSLSPGERATLSCRASQSVSSYLAWY |
| | Variable | QQKPGQAPRLLIYDASNRATGIPARFSGSGSGTDFT |
| | | LTISSLEPEDFAVYYCQQSSNWPRTFGQGTKVEIK |
| 35 | Anti-hPD-1-A Heavy Chain | QVQLVESGGGVVQPGRSLRLDCKASGITFSNSGMH |
| | Variable | WVRQAPGKGLEWVAVIWYDGSKRYYADSVKGRF |
| | | TISRDNSKNTLFLQMNSLRAEDTAVYYCATNDDY |
| | | WGQGTLVTVSS |
| 36 | Anti-hPD-1-A Light Chain | EIVLTQSPATLSLSPGERATLSCRASQSVSSYLAWY |
| | | QQKPGQAPRLLIYDASNRATGIPARFSGSGSGTDFT |
| | | LTISSLEPEDFAVYYCQQSSNWPRTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 37 | Anti-hPD-1-A Heavy Chain | QVQLVESGGGVVQPGRSLRLDCKASGITFSNSGMH |
| | | WVRQAPGKGLEWVAVIWYDGSKRYYADSVKGRF |
| | | TISRDNSKNTLFLQMNSLRAEDTAVYYCATNDDYW |
| | | GQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDT |
| | | LMISRTPEVTCVVVDVSQEDPEVQFNWYVDGVEVH |
| | | NAKTKPREEQFNSTYRVVSVLTVLHQDWLNGKEYK |
| | | CKVSNKGLPSSIEKTISKAKGQPREPQVYTLPPSQ |
| | | EEMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGK |
| 38 | Anti-hPD-1-B CDRL1 | KGVSTSGYSY |
| | (IMGT) | |
| | Anti-hPD-1-B CDRL2 | LAS |
| | (IMGT) | |
| 39 | Anti-hPD-1-B CDRL3 | QHSRDLPLT |
| | (IMGT) | |
| 40 | Anti-hPD-1-B CDRH1 | GYTFTNYY |
| | (IMGT) | |
| 41 | Anti-hPD-1-B CDRH2 | INPSNGGT |
| | (IMGT) | |
| 42 | Anti-hPD-1-B CDRH3 | ARRDYRFDMGFDY |
| | (IMGT) | |
| 43 | Anti-hPD-1-B Light Chain | EIVLTQSPATLSLSPGERATLSCRASKGVSTSGYSY |
| | Variable | LHWYQQKPGQAPRLLIYLASYLESGVPARFSGSGSG |
| | | TDFTLTISSLEPEDFAVYYCQHSRDLPLTFGGGTKV |
| | | EIK |
| 44 | Anti-hPD-1-B Heavy Chain | QVQLVQSGVEVKKPGASVKVSCKASGYTFTNYYM |
| | Variable | YWVRQAPGQGLEWMGGINPSNGGTNFNEKFKNR |
| | | VTLTTDSSTTTAYMELKSLQFDDTAVYYCARRDY |
| | | RFDMGFDYWGQGTTVTVSS |
| 45 | Anti-hPD-1-B Light Chain | EIVLTQSPATLSLSPGERATLSCRASKGVSTSGYSY |
| | | LHWYQQKPGQAPRLLIYLASYLESGVPARFSGSGSG |
| | | TDFTLTISSLEPEDFAVYYCQHSRDLPLTFGGGTKV |
| | | EIKRTVAAPSVFIFPPSDEQLKSGTASVVCLLNNFY |
| | | PREAKVQWKVDNALQSGNSQESVTEQDSKDSTYSLS |
| | | STLTLSKADYEKHKVYACEVTHQGLSSPVTKSFN |
| | | RGEC |
| 46 | Anti-hPD-1-B Heavy Chain | QVQLVQSGVEVKKPGASVKVSCKASGYTFTNYYM |
| | | YWVRQAPGQGLEWMGGINPSNGGTNFNEKFKNR |
| | | VTLTTDSSTTTAYMELKSLQFDDTAVYYCARRDY |
| | | RFDMGFDYWGQGTTVTVSSASTKGPSVFPLAPCSR |
| | | STSESTAALGCLVKDYFPEPVTVSWNSGALTSGVH |
| | | TFPAVLQSSGLYSLSSVVTVPSSSLGTKTYTCNVDH |
| | | KPSNTKVDKRVESKYGPPCPPCPAPEFLGGPSVFLF |
| | | PPKPKDTLMISRTPEVTCVVVDVSQEDPEVQFNWY |
| | | VDGVEVHNAKTKPREEQFNSTYRVVSVLTVLHQD |
| | | WLNGKEYKCKVSNKGLPSSIEKTISKAKGQPREPQ |
| | | VYTLPPSQEEMTKNQVSLTCLVKGFYPSDIAVEWE |
| | | SNGQPENNYKTTPPVLDSDGSFFLYSRLTVDKSRW |
| | | QEGNVFSCSVMHEALHNHYTQKSLSLSLGK |
| 47 | Anti-hPD-1-C CDRL1 | QSIVHSNGNTY |
| | (IMGT) | |
| | Anti-hPD-1-C CDRL2 | KVS |
| | (IMGT) | |
| 48 | Anti-hPD-1-C CDRL3 | FQGSHVPLT |
| | (IMGT) | |
| 49 | Anti-hPD-1-C CDRH1 | GYTFTDYE |
| | (IMGT) | |
| 50 | Anti-hPD-1-C CDRH2 | IESETGGT |
| | (IMGT) | |
| 51 | Anti-hPD-1-C CDRH3 | AREGITTVATTYYWYFDV |
| | (IMGT) | |
| 52 | Anti-hPD-1-C Light Chain | DVVMTQSPLSLPVTLGQPASISCRSSQSIVHSNGNT |
| | Variable | YLEWYLQKPGQSPQLLIYKVSNRFSGVPDRFSGSG |
| | | SGTDFTLKISRVEAEDVGVYYCFQGSHVPLTFGQG |
| | | TKLEIK |
| 53 | Anti-hPD-1-C Heavy Chain | QGQLVQSGAEVKKPGASVKVSCKASGYTFTDYEM |
| | Variable | HWVRQAPIHGLEWIGVIESETGGTAYNQKFKGRVT |
| | | ITADKSTSTAYMELSSLRSEDTAVYYCAREGITTV |
| | | ATTYYWYFDVWGQGTTVTVSS |
| 54 | Anti-hPD-1-C Light Chain | DVVMTQSPLSLPVTLGQPASISCRSSQSIVHSNGNT |
| | (Kappa) | YLEWYLQKPGQSPQLLIYKVSNRFSGVPDRESGSG |
| | | SGTDFTLKISRVEAEDVGVYYCFQGSHVPLTFGQG |
| | | TKLEIKRTVAAPSVFIFPPSDEQLKSGTASVVCLLN |
| | | NFYPREAKVQWKVDNALQSGNSQESVTEQDSKDS |
| | | TYSLSSTLTLSKADYEKHKVYACEVTHQGLSSPVT |
| | | KSFNRGEC |
| 55 | Anti-hPD-1-C Heavy Chain | QGQLVQSGAEVKKPGASVKVSCKASGYTFTDYEM |
| | | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLTCLVKGFYPSDIAVEW |
| | | ESNGQPENNYKTTPPVLDSDGSFFLYSRLTVDKSR |
| | | WQEGNVFSCSVMHEALHNHYTQKSLSLSLGK |
| 56 | Anti-hPD-1-D CDRL1 | QTIGTW |
| | (IMGT) | |
| | Anti-hPD-1-D CDRL2 | TAT |
| | (IMGT) | |
| 57 | Anti-hPD-1-D CDRL3 | QQVYSIPWT |
| | (IMGT) | |
| 58 | Anti-hPD-1-D CDRH1 | GFTFSSYM |
| | (IMGT) | |
| 59 | Anti-hPD-1-D CDRH2 | ISGGGANT |
| | (IMGT) | |
| 60 | Anti-hPD-1-D CDRH3 | ARQLYYFDY |
| | (IMGT) | |
| 61 | Anti-hPD-1-D Light Chain | DIQMTQSPSSLSASVGDRVTITCLASQTIGTWLTWY |
| | Variable | QQKPGKAPKLLIYTATSLADGVPSRFSGSGSGTDFT |
| | | LTISSLQPEDFATYYCQQVYSIPWTFGGGTKVEIK |
| 62 | Anti-hPD-1-D Heavy Chain | EVQLVESGGGLVQPGGSLRLSCAASGFTFSSYMMS |
| | Variable | WVRQAPGKGLEWVATISGGGANTYYPDSVKGRFT |
| | | ISRDNAKNSLYLQMNSLRAEDTAVYYCARQLYYF |
| | | DYWGQGTTVTVSS |
| 63 | Anti-hPD-1-D Light Chain | DIQMTQSPSSLSASVGDRVTITCLASQTIGTWLTWY |
| | | QQKPGKAPKLLIYTATSLADGVPSRFSGSGSGTDFT |
| | | LTISSLQPEDFATYYCQQVYSIPWTFGGGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 64 | Anti-hPD-1-D Heavy Chain | EVQLVESGGGLVQPGGSLRLSCAASGFTFSSYMMS |
| | | WVRQAPGKGLEWVATISGGGANTYYPDSVKGRFT |
| | | ISRDNAKNSLYLQMNSLRAEDTAVYYCARQLYYF |
| | | DYWGQGTTVTVSSASTKGPSVFPLAPCSRSTSESTA |
| | | ALGCLVKDYFPEPVTVSWNSGALTSGVHTFPAVLQ |
| | | SSGLYSLSSVVTVPSSSLGTKTYTCNVDHKPSNTKV |
| | | DKRVESKYGPPCPPCPAPEFLGGPSVFLFPPKPKDT |
| | | LMISRTPEVTCVVVDVSQEDPEVQFNWYVDGVEV |
| | | HNAKTKPREEQFNSTYRVVSVLTVLHQDWLNGKE |
| | | YKCKVSNKGLPSSIEKTISKAKGQPREPQVYTLPPS |
| | | QEEMTKNQVSLTCLVKGFYPSDIAVEWESNGQPEN |
| | | NYKTTPPVLDSDGSFFLYSRLTVDKSRWQEGNVFS |
| | | CSVMHEALHNHYTQKSLSLSLGK |
| 65 | Anti-hPD-1-E CDRL1 | QGISSW |
| | (IMGT) | |
| | Anti-hPD-1-E CDRL2 | AAS |
| | (IMGT) | |
| 66 | Anti-hPD-1-E CDRL3 | QQANHLPFT |
| | (IMGT) | |
| 67 | Anti-hPD-1-E CDRH1 | GGTFSSYA |
| | (IMGT) | |
| 68 | Anti-hPD-1-E CDRH2 | IIPMFDTA |
| | (IMGT) | |
| 69 | Anti-hPD-1-E CDRH3 | ARAEHSSTGTFDY |
| | (IMGT) | |
| 70 | Anti-hPD-1-E Light Chain | DIQMTQSPSSVSASVGDRVTITCRASQGISSWLAW |
| | Variable | YQQKPGKAPKLLISAASSLQSGVPSRFSGSGSGTDF |
| | | TLTISSLQPEDFATYYCQQANHLPFTFGGGTKVEIK |
| 71 | Anti-hPD-1-E Heavy Chain | QVQLVQSGAEVKKPGSSVKVSCKASGGTFSSYAIS |
| | Variable | WVRQAPGQGLEWMGLIIPMFDTAGYAQKFQGRV |
| | | AITVDESTSTAYMELSSLRSEDTAVYYCARAEHSST |
| | | GTFDYWGQGTLVTVSS |
| 72 | Anti-hPD-1-E Light Chain | DIQMTQSPSSVSASVGDRVTITCRASQGISSWLAW |
| | | YQQKPGKAPKLLISAASSLQSGVPSRFSGSGSGTDF |
| | | TLTISSLQPEDFATYYCQQANHLPFTFGGGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 73 | Anti-hPD-1-E Heavy Chain | QVQLVQSGAEVKKPGSSVKVSCKASGGTFSSYAIS |
| | | WVRQAPGQGLEWMGLIIPMFDTAGYAQKFQGRV |
| | | AITVDESTSTAYMELSSLRSEDTAVYYCARAEHSST |
| | | GTFDYWGQGTLVTVSSASTKGPSVFPLAPCSRSTSE |
| | | STAALGCLVKDYFPEPVTVSWNSGALTSGVHTFPA |
| | | VLQSSGLYSLSSVVTVPSSSLGTKTYTCNVDHKPSN |
| | | TKVDKRVESKYGPPCPPCPAPEFLGGPSVFLFPPKP |
| | | KDTLMISRTPEVTCVVVDVSQEDPEVQFNWYVDG |
| | | VEVHNAKTKPREEQFNSTYRVVSVLTVLHQDWLN |
| | | GKEYKCKVSNKGLPSSIEKTISKAKGQPREPQVYTL |
| | | PPSQEEMTKNQVSLTCLVKGFYPSDIAVEWESNGQ |
| | | PENNYKTTPPVLDSDGSFFLYSRLTVDKSRWQEGN |
| | | VFSCSVMHEALHNHYTQKSLSLSLGK |
| 74 | Anti-hPD-1-F CDRL1 | QSLLDSGNQKNF |
| | (IMGT) | |
| | Anti-hPD-1-F CDRL2 | WAS |
| | (IMGT) | |
| 75 | Anti-hPD-1-F CDRL3 | QNDYSYPYT |
| | (IMGT) | |
| 76 | Anti-hPD-1-F CDRH1 | GYTFTTYW |
| | (IMGT) | |
| 77 | Anti-hPD-1-F CDRH2 | IYPGTGGS |
| | (IMGT) | |
| 78 | Anti-hPD-1-F CDRH3 | TRWTTGTGAY |
| | (IMGT) | |
| 79 | Anti-hPD-1-F Light Chain | EIVLTQSPATLSLSPGERATLSCKSSQSLLDSGNQK |
| | Variable | NFLTWYQQKPGQAPRLLIYWASTRESGVPSRFSGS |
| | | GSGTDFTETISSLEAEDAATYYCQNDYSYPYTFGQ |
| | | GTKVEIK |
| 80 | Anti-hPD-1-F Heavy Chain | EVQLVQSGAEVKKPGESLRISCKGSGYTFTTYWMH |
| | Variable | WVRQATGQGLEWMGNIYPGTGGSNFDEKEKNRV |
| | | TITADKSTSTAYMELSSLRSEDTAVYYCTRWTTGT |
| | | GAYWGQGTTVTVSS |
| 81 | Anti-hPD-1-F Light Chain | EIVLTQSPATLSLSPGERATLSCKSSQSLLDSGNQK |
| | | NFLTWYQQKPGQAPRLLIYWASTRESGVPSRESGS |
| | | GSGTDFTETISSLEAEDAATYYCQNDYSYPYTFGQ |
| | | GTKVEIKRTVAAPSVFIFPPSDEQLKSGTASVVCLL |
| | | NNFYPREAKVQWKVDNALQSGNSQESVTEQDSKD |
| | | STYSLSSTLTLSKADYEKHKVYACEVTHQGLSSPV |
| | | TKSFNRGEC |
| 82 | Anti-hPD-1-F Heavy Chain | EVQLVQSGAEVKKPGESLRISCKGSGYTFTTYWMH |
| | | WVRQATGQGLEWMGNIYPGTGGSNFDEKEKNRV |
| | | TITADKSTSTAYMELSSLRSEDTAVYYCTRWTTGT |
| | | GAYWGQGTTVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNTK |
| | | VDKRVESKYGPPCPPCPAPEFLGGPSVFLFPPKPKD |
| | | TLMISRTPEVTCVVVDVSQEDPEVQFNWYVDGVE |
| | | VHNAKTKPREEQFNSTYRVVSVLTVLHQDWLNGK |
| | | EYKCKVSNKGLPSSIEKTISKAKGQPREPQVYTLPP |
| | | SQEEMTKNQVSLTCLVKGFYPSDIAVEWESNGQPE |
| | | NNYKTTPPVLDSDGSFFLYSRLTVDKSRWQEGNVF |
| | | SCSVMHEALHNHYTQKSLSLSLQG |
| 83 | Anti-hPD-1-G CDRL1 | ESVSND |
| | (IMGT) | |
| | Anti-hPD-1-G CDRL2 | YAF |
| | (IMGT) | |
| 84 | Anti-hPD-1-G CDRL3 | HQAYSSPYT |
| | (IMGT) | |
| 85 | Anti-hPD-1-G CDRH1 | GFSLTSYG |
| | (IMGT) | |
| 86 | Anti-hPD-1-G CDRH2 | IYADGST |
| | (IMGT) | |
| 87 | Anti-hPD-1-G CDRH3 | ARAYGNYWYIDV |
| | (IMGT) | |
| 88 | Anti-hPD-1-G Light Chain | DIVMTQSPDSLAVSLGERATINCKSSESVSNDVAW |
| | Variable | YQQKPGQPPKLLINYAFHRFTGVPDRFSGSGYGTD |
| | | FTLTISSLQAEDVAVYYCHQAYSSPYTFGQGTKLEI |
| | | K |
| 89 | Anti-hPD-1-G Heavy Chain | QVQLQESGPGLVKPSETLSLTCTVSGFSLTSYGVH |
| | Variable | WIRQPPGKGLEWIGVIYADGSTNYNPSLKSRVTISK |
| | | DTSKNQVSLKLSSVTAADTAVYYCARAYGNYWYI |
| | | DVWGQGTTVTVSS |
| 90 | Anti-hPD-1-G Light Chain | DIVMTQSPDSLAVSLGERATINCKSSESVSNDVAW |
| | | YQQKPGQPPKLLINYAFHRFTGVPDRFSGSGYGTD |
| | | FTLTISSLQAEDVAVYYCHQAYSSPYTFGQGTKLEI |
| | | KRTVAAPSVFIFPPSDEQLKSGTASVVCLLNNFYPR |
| | | EAKVQWKVDNALQSGNSQESVTEQDSKDSTYSLS |
| | | STLTLSKADYEKHKVYACEVTHQGLSSPVTKSFNR |
| | | GEC |
| 91 | Anti-hPD-1-G Heavy Chain | QVQLQESGPGLVKPSETLSLTCTVSGFSLTSYGVH |
| | | WIRQPPGKGLEWIGVIYADGSTNYNPSLKSRVTISK |
| | | DTSKNQVSLKLSSVTAADTAVYYCARAYGNYWYI |
| | | DVWGQGTTVTVSSASTKGPSVFPLAPCSRSTSESTA |
| | | ALGCLVKDYFPEPVTVSWNSGALTSGVHTFPAVLQ |
| | | SSGLYSLSSVVTVPSSSLGTKTYTCNVDHKPSNTKV |
| | | DKRVESKYGPPCPPCPAPPVAGGPSVFLFPPKPKDT |
| | | LMISRTPEVTCVVVAVSQEDPEVQENWYVDGVEV |
| | | HNAKTKPREEQENSTYRVVSVLTVVHQDWLNGKE |
| | | YKCKVSNKGLPSSIEKTISKAKGQPREPQVYTLPPS |
| | | QEEMTKNQVSLTCLVKGFYPSDIAVEWESNGQPEN |
| | | NYKTTPPVLDSDGSFFLYSKLTVDKSRWQEGNVFS |
| | | CSVMHEALHNHYTQKSLSLSLGIK |
| 92 | Anti-hPD-1-H CDRL1 | LSINTF |
| | (IMGT) | |
| | Anti-hPD-1-H CDRL2 | AAS |
| | (IMGT) | |
| 93 | Anti-hPD-1-H CDRL3 | QQSSNTPFT |
| | (IMGT) | |
| 94 | Anti-hPD-1-H CDRH1 | GFTFSNFG |
| | (IMGT) | |
| 95 | Anti-hPD-1-H CDRH2 | ISGGGRDT |
| | (IMGT) | |
| 96 | Anti-hPD-1-H CDRH3 | VKWGNIYFDY |
| | (IMGT) | |
| 97 | Anti-hPD-1-H Light Chain | DIQMTQSPSSLSASVGDSITITCRASLSINTFLNWYQ |
| | Variable | QKPGKAPNLLIYAASSLHGGVPSRFSGSGSGTDFTL |
| | | TIRTLQPEDFATYYCQQSSNTPFTFGPGTVVDFR |
| 98 | Anti-hPD-1-H Heavy Chain | EVQLLESGGVLVQPGGSLRLSCAASGFTFSNFGMT |
| | Variable | WVRQAPGKGLEWVSGISGGGRDTYFADSVKGRFT |
| | | ISRDNSKNTLYLQMNSLKGEDTAVYYCVKWGNIY |
| | | FDYWGQGTLVTVSS |
| 99 | Anti-hPD-1-H Light Chain | DIQMTQSPSSLSASVGDSITITCRASLSINTFLNWYQ |
| | | QKPGKAPNLLIYAASSLHGGVPSRFSGSGSGTDFTL |
| | | TIRTLQPEDFATYYCQQSSNTPFTFGPGTVVDFRRT |
| | | VAAPSVFIFPPSDEQLKSGTASVVCLLNNFYPREAK |
| | | VQWKVDNALQSGNSQESVTEQDSKDSTYSLSSTLT |
| | | LSKADYEKHKVYACEVTHQGLSSPVTKSFNRGEC |
| 100 | Anti-hPD-1-H Heavy Chain | EVQLLESGGVLVQPGGSLRLSCAASGFTFSNFGMT |
| | | WVRQAPGKGLEWVSGISGGGRDTYFADSVKGRFT |
| | | ISRDNSKNTLYLQMNSLKGEDTAVYYCVKWGNIY |
| | | FDYWGQGTLVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNTK |
| | | VDKRVESKYGPPCPPCPAPEFLGGPSVFLFPPKPKD |
| | | TLMISRTPEVTCVVVDVSQEDPEVQFNWYVDGVE |
| | | VHNAKTKPREEQFNSTYRVVSVLTVLHQDWLNGK |
| | | EYKCKVSNKGLPSSIEKTISKAKGQPREPQVYTLPP |
| | | SQEEMTKNQVSLTCLVKGFYPSDIAVEWESNGQPE |
| | | NNYKTTPPVLDSDGSFFLYSRLTVDKSRWQEGNVF |
| | | SCSVMHEALHNHYTQKSLSLSLGK |
| 101 | Anti-hPD-1-I CDRL1 | QDVTTA |
| | (IMGT) | |
| | Anti-hPD-1-I CDRL2 | WAS |
| | (IMGT) | |
| 102 | Anti-hPD-1-I CDRL3 | QQHYTIPWT |
| | (IMGT) | |
| 103 | Anti-hPD-1-I CDRH1 | GFTFSNYG |
| | (IMGT) | |
| 104 | Anti-hPD-1-I CDRH2 | ISGGGSNI |
| | (IMGT) | |
| 105 | Anti-hPD-1-I CDRH3 | VSYYYGIDF |
| | (IMGT) | |
| 106 | Anti-hPD-1-I Light Chain | DIQMTQSPSSLSASVGDRVTITCKASQDVTTAVAW |
| | Variable | YQQKPGKAPKLLIYWASTRHTGVPSRFSGSGSGTD |
| | | FTLTISSLQPEDFATYYCQQHYTIPWTFGGGTKLEI |
| | | K |
| 107 | Anti-hPD-1-I Heavy Chain | QVQLVESGGGLVKPGGSLRLSCAASGFTFSNYGMS |
| | Variable | WIRQAPGKGLEWVSTISGGGSNIYYADSVKGRFTIS |
| | | RDNAKNSLYLQMNSLRAEDTAVYYCVSYYYGIDF |
| | | WGQGTSVTVSS |
| 108 | Anti-hPD-1-I Light Chain | DIQMTQSPSSLSASVGDRVTITCKASQDVTTAVAW |
| | | YQQKPGKAPKLLIYWASTRHTGVPSRFSGSGSGTD |
| | | FTLTISSLQPEDFATYYCQQHYTIPWTFGGGTKLEI |
| | | KRTVAAPSVFIFPPSDEQLKSGTASVVCLLNNFYPR |
| | | EAKVQWKVDNALQSGNSQESVTEQDSKDSTYSLS |
| | | STLTLSKADYEKHKVYACEVTHQGLSSPVTKSFNR |
| | | GEC |
| 109 | Anti-hPD-1-I Heavy Chain | QVQLVESGGGLVKPGGSLRLSCAASGFTFSNYGMS |
| | | WIRQAPGKGLEWVSTISGGGSNIYYADSVKGRFTIS |
| | | RDNAKNSLYLQMNSLRAEDTAVYYCVSYYYGIDF |
| | | WGQGTSVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGK |
| 110 | Anti-hPD-1-J CDRL1 | ESVDNYGMSF |
| | (IMGT) | |
| | Anti-hPD-1-J CDRL2 | AAS |
| | (IMGT) | |
| 111 | Anti-hPD-1-J CDRL3 | QQSKEVPYT |
| | (IMGT) | |
| 112 | Anti-hPD-1-J CDRH1 | GYSFTSYW |
| | (IMGT) | |
| 113 | Anti-hPD-1-J CDRH2 | IHPSDSET |
| | (IMGT) | |
| 114 | Anti-hPD-1-J CDRH3 | AREHYGTSPFAY |
| | (IMGT) | |
| 115 | Anti-hPD-1-J Light Chain | EIVLTQSPATLSLSPGERATLSCRASESVDNYGMSF |
| | Variable | MNWFQQKPGQPPKLLIHAASNQGSGVPSRFSGSGS |
| | | GTDFTLTISSLEPEDFAVYFCQQSKEVPYTFGGGTK |
| | | VEIK |
| 116 | Anti-hPD-1-J Heavy Chain | QVQLVQSGAEVKKPGASVKVSCKASGYSFTSYWM |
| | Variable | NWVRQAPGQGLEWIGVIHPSDSETWLDQKFKDRV |
| | | TITVDKSTSTAYMELSSLRSEDTAVYYCAREHYGT |
| | | SPFAYWGQGTLVTVSS |
| 117 | Anti-hPD-1-J Light Chain | EIVLTQSPATLSLSPGERATLSCRASESVDNYGMSF |
| | | MNWFQQKPGQPPKLLIHAASNQGSGVPSRFSGSGS |
| | | GTDFTLTISSLEPEDFAVYFCQQSKEVPYTFGGGTK |
| | | VEIKRTVAAPSVFIFPPSDEQLKSGTASVVCLLNNF |
| | | YPREAKVQWKVDNALQSGNSQESVTEQDSKDSTY |
| | | SLSSTLTLSKADYEKHKVYACEVTHQGLSSPVTKS |
| | | FNRGEC |
| 118 | Anti-hPD-1-J Heavy Chain | QVQLVQSGAEVKKPGASVKVSCKASGYSFTSYWM |
| | | NWVRQAPGQGLEWIGVIHPSDSETWLDQKFKDRV |
| | | TITVDKSTSTAYMELSSLRSEDTAVYYCAREHYGT |
| | | SPFAYWGQGTLVTVSSASTKGPSVFPLAPCSRSTSE |
| | | STAALGCLVKDYFPEPVTVSWNSGALTSGVHTFPA |
| | | VLQSSGLYSLSSVVTVPSSSLGTKTYTCNVDHKPSN |
| | | TKVDKRVESKYGPPCPPCPAPEFLGGPSVFLFPPKP |
| | | KDTLMISRTPEVTCVVVDVSQEDPEVQFNWYVDG |
| | | VEVHNAKTKPREEQFNSTYRVVSVLTVLHQDWLN |
| | | GKEYKCKVSNKGLPSSIEKTISKAKGQPREPQVYTL |
| | | PPSQEEMTKNQVSLTCLVKGFYPSDIAVEWESNGQ |
| | | PENNYKTTPPVLDSDGSFFLYSRLTVDKSRWQEGN |
| | | VFSCSVMHEALHNHYTQKSLSLSLG |
| 119 | Anti-hPD-1-V_{H}H-A CDR1 | GSRIA |
| | (IMGT) | |
| 120 | Anti-hPD-1-V_{H}H-A CDR2 | ISQNGGST |
| | (IMGT) | |
| 121 | Anti-hPD-1-V_{H}H-A CDR3 | AAALMTWYGTSYCPHLSTEFGF |
| | (IMGT) | |
| 122 | Anti-hPD-1-V_{H}H-A | QVKLEESGGGSVQAGGSLRLSCAASGSRIAMGWF |
| | | RQAPGKEREFVGAISQNGGSTCYADSVKDRFTISR |
| | | DNAKNMVYLQMNSLKPEDTAVYYCAAALMTWY |
| | | GTSYCPHLSTEFGFWGQGTLVTVSS |
| 123 | Anti-hPD-1-V_{H}H-B CDR1 | GFTLDDYA |
| | (IMGT) | |
| 124 | Anti-hPD-1-V_{H}H-B CDR2 | ISSSDGST |
| | (IMGT) | |
| 125 | Anti-hPD-1-V_{H}H-B CDR3 | STDVGCYGSTSPEAY |
| | (IMGT) | |
| 126 | Anti-hPD-1-V_{H}H-B | QVKLEESGGGLVQPGGSLRLSCAASGFTLDDYALG |
| | | WFRQAPGKEREGVSCISSSDGSTYYADSVKGRFTIS |
| | | RDNAKNTVYLQMNSLKPEDTAVYYCSTDVGCYGS |
| | | TSPEAYWGKGTLVTVSS |
| 127 | Anti-hPD-1-V_{H}H-C CDR1 | GRTFSSYD |
| | (IMGT) | |
| 128 | Anti-hPD-1-V_{H}H-C CDR2 | VSASGGRS |
| | (IMGT) | |
| 129 | Anti-hPD-1-V_{H}H-C CDR3 | AARNGAYYITIDPAQYNY |
| | (IMGT) | |
| 130 | Anti-hPD-1-V_{H}H-C | QVQLVDSGGGLVQAGGSLRLSCAASGRTFSSYDFG |
| | | WFRQAPGKEREFVAAVSASGGRSGYSDFAKDRFT |
| | | TSRENAKNTVYLQMNSLKPEDTAVYYCAARNGAY |
| | | YITIDPAQYNYWGQGTLVTVSS |
| 131 | Anti-hPD-1-V_{H}H-D CDR1 | GRTFSSYA |
| | (IMGT) | |
| 132 | Anti-hPD-1-V_{H}H-D CDR2 | ISWSGGIT |
| | (IMGT) | |
| 133 | Anti-hPD-1-V_{H}H-D CDR3 | AADLNQESSWYDY |
| | (IMGT) | |
| 134 | Anti-hPD-1-V_{H}H-D | QVQLVESGGGLVQAGDSLTLSCAASGRTFSSYAM |
| | | GWFRQAPGKEREFVAAISWSGGITYYEDSVKGRFT |
| | | ISRDNAKNTVYLQMNSLKPEDTAVYYCAADLNQE |
| | | SSWYDYWGQGTQVTVSS |
| 135 | Anti-hPD-1-V_{H}H-E CDR1 | GSRYA |
| | (IMGT) | |
| 136 | Anti-hPD-1-V_{H}H-E CDR2 | ISWNGGST |
| | (IMGT) | |
| 137 | Anti-hPD-1-V_{H}H-E CDR3 | AAALIPKYGTSYCPHISTEFDF |
| | (IMGT) | |
| 138 | Anti-hPD-1-V_{H}H-E | QVKLEESGGGSVQAGGSLRLSCAGSGSRYAMGWF |
| | | RQAPGKEREFVAAISWNGGSTCYADSVKGRFTISR |
| | | DNAKNMVYLQMNSLKPEDTAVYYCAAALIPKYGT |
| | | SYCPHISTEFDFWGQGTQVTVSS |
| | GSG Linker | GSG |
| 139 | (GSG)₂ Linker | GSGGSG |
| 140 | (GSG)₃ Linker | GSGGSGGSG |
| 141 | (GSG)₄ Linker | GSGGSGGSGGSG |
| 142 | G3S Linker | GGGS |
| 143 | (G3S)₂ Linker | GGGSGGGS |
| 144 | (G3S)₃ Linker | GGGSGGGSGGGS |
| 145 | (G3S)₄ Linker | GGGSGGGSGGGSGGGS |
| 146 | G4S Linker | GGGGS |
| 147 | (G4S)₂ Linker | GGGGSGGGGS |
| 148 | (G4S)₃ Linker | GGGGSGGGGSGGGGS |
| 149 | (G4S)₄ Linker | GGGGSGGGGSGGGGSGGGGS |
| 150 | SGSG Linker | SGSG |
| 151 | S(GSG)₂ Linker | SGSGGSG |
| 152 | S(GSG)₃ Linker | SGSGGSGGSG |
| 153 | S(GSG)₄ Linker | SGSGGSGGSGGSG |
| 154 | SG3S Linker | SGGGS |
| 155 | S(G3S)₂ Linker | SGGGSGGGS |
| 156 | S(G3S)₃ Linker | SGGGSGGGSGGGS |
| 157 | S(G3S)₄ Linker | SGGGSGGGSGGGSGGGS |
| 158 | SG4S Linker | SGGGGS |
| 159 | S(G4S)₂ Linker | SGGGGSGGGGS |
| 160 | S(G4S)₃ Linker | SGGGGSGGGGSGGGGS |
| 161 | S(G4S)₄ Linker | SGGGGSGGGGSGGGGSGGGGS |
| 162 | EAAAK Linker | EAAAK |
| 163 | (EAAAK)₂ Linker | EAAAKEAAAK |
| 164 | (EAAAK)₃ Linker | EAAAKEAAAKEAAAK |
| 165 | (EAAAK)₄ Linker | EAAAKEAAAKEAAAKEAAAK |
| 166 | PAPAP Linker | PAPAP |
| 167 | (PAPAP)₂ Linker | PAPAPPAPAP |
| 168 | (PAPAP)₃ Linker | PAPAPPAPAPPAPAP |
| 169 | (PAPAP)₄ Linker | PAPAPPAPAPPAPAPPAPAP |
| 170 | VLVH Linker | IKRTVAAP |
| 171 | RAKPS Linker | RAKPS |
| 172 | (RAKPS)₂ Linker | RAKPSRAKPS |
| 173 | ASTKG Linker | ASTKG |
| 174 | (ASTKG)₂ Linker | ASTKGASTKG |
| 175 | AKTHT Linker | AKTHT |
| 176 | (AKTHT)₂ Linker | AKTHTAKTHT |
| 177 | ASTKG(G4S)₂ Linker | ASTKGGGGGSGGGGS |
| 178 | RAKPS(G4S)₂ Linker | RAKPSGGGGSGGGGS |
| 179 | hIL-2 | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFCQSIISTLT |
| 180 | hIL-2 (T3A) | APASSSTKKTQLQLEHLLLDLQMILNGINNYKNPK |
| | | LTRMLTFKFYMPKKATELKHLQCLEEELKPLEEVL |
| | | NLAQSKNFHLRPRDLISNINVIVLELKGSETTFMCE |
| | | YADETATIVEFLNRWITFCQSIISTLT |
| 181 | hIL-2 (C125S) | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 182 | hIL-2 (C125A) | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFAQSIISTLT |
| 183 | hIL-2 (T3A, C125S) | APASSSTKKTQLQLEHLLLDLQMILNGINNYKNPK |
| | | LTRMLTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 184 | hIL-2 (T3A, C125A) | APASSSTKKTQLQLEHLLLDLQMILNGINNYKNPK |
| | | LTRMLTFKFYMPKKATELKHLQCLEEELKPLEEVL |
| | | NLAQSKNFHLRPRDLISNINVIVLELKGSETTFMCE |
| | | YADETATIVEFLNRWITFAQSIISTLT |
| 185 | hIL-2 Mutein | APTSSSTKKTQLQLEHLLLDLQMILNGINNYKNPKL |
| | (R38N, L40T, C125S) | TNMTTFKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 186 | hIL-2 Mutein | APASSSTKKTQLQLEHLLLDLQMILNGINNYKNPK |
| | (T3A, R38N, L40T, C125S) | LTNMTTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 187 | hIL-2 Mutein | APTSSSTKKTQLQLKHLLLDLQMILNGINNYKNPK |
| | (E15K, R38N, L40T, C125S) | LTNMTTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 188 | hIL-2 Mutein | APASSSTKKTQLQLKHLLLDLQMILNGINNYKNPK |
| | (T3A, E15K, R38N, L40T, | LTNMTTFKFYMPKKATELKHLQCLEEELKPLEEVL |
| | C125S) | NLAQSKNFHLRPRDLISNINVIVLELKGSETTFMCE |
| | | YADETATIVEFLNRWITFSQSIISTLT |
| 189 | hIL-2 Mutein | APTSSSTKKTQLQLKHLLLDLQMILNGINNYENPKL |
| | (E15K, K32E, R38N, L40T, | TNMTTFKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 190 | hIL-2 Mutein | APASSSTKKTQLQLKHLLLDLQMILNGINNYENPK |
| | (T3A, E15K, K32E, R38N, | LTNMTTFKFYMPKKATELKHLQCLEEELKPLEEVL |
| | L40T, C125S) | NLAQSKNFHLRPRDLISNINVIVLELKGSETTFMCE |
| | | YADETATIVEFLNRWITFSQSIISTLT |
| 191 | hIL-2 Mutein | APTSSSTKKTQLQLKHLLLDLQMILNGINNYKNPK |
| | (E15K, R38N, L40T, K76E, | LTNMTTFKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 192 | hIL-2 Mutein | APASSSTKKTQLQLKHLLLDLQMILNGINNYKNPK |
| | (T3A, E15K, R38N, L40T, | LTNMTTFKFYMPKKATELKHLQCLEEELKPLEEVL |
| | K76E, C125S) | NLAQSENFHLRPRDLISNINVIVLELKGSETTFMCE |
| | | YADETATIVEFLNRWITFSQSIISTLT |
| 193 | hIL-2 Mutein | APTSSSTKKTQLQLEHLLLDLQMILNGINNYENPKL |
| | (K32E, R38E, F42A, C125S) | TEMLTAKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 194 | hIL-2 Mutein | APASSSTKKTQLQLEHLLLDLQMILNGINNYENPKL |
| | (T3A, K32E, R38E, F42A, | TEMLTAKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 195 | hIL-2 Mutein | APTSSSTKKTQLQLEHLLLDLQMILNGINNYENPKL |
| | (K32E, R38E, F42K, C125S) | TEMLTKKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 196 | hIL-2 Mutein | APASSSTKKTQLQLEHLLLDLQMILNGINNYENPKL |
| | (T3A, K32E, R38E, F42K, | TEMLTKKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 197 | hIL-2 Mutein | APTSSSTKKTQLQLKHLLLDLQMILNGINNYENPKL |
| | (E15K, K32E, R38E, F42K, | TEMLTKKFYMPKKATELKHLQCLEEELKPLEEVLN |
| | C125S) | LAQSKNFHLRPRDLISNINVIVLELKGSETTFMCE |
| | | YADETATIVEFLNRWITFSQSIISTLT |
| 198 | hIL-2 Mutein | APASSSTKKTQLQLKHLLLDLQMILNGINNYENPK |
| | (T3A, E15K, K32E, R38E, | LTEMLTKKFYMPKKATELKHLQCLEEELKPLEEVL |
| | F42K, C125S) | NLAQSKNFHLRPRDLISNINVIVLELKGSETTFMC |
| | | EYADETATIVEFLNRWITFSQSIISTLT |
| 199 | hIL-2 Mutein | APTSSSTKKTQLQLEHLLLDLQMILNGINNYKNPKL |
| | (R38E, F42A, K76E, C125S) | TEMLTAKFYMPKKATELKHLQCLEEELKPLEEVLN |
| | | LAQSENFHLRPRDLISNINVIVLELKGSETTFMCE |
| | | YADETATIVEFLNRWITFSQSIISTLT |
| 200 | hIL-2 Mutein | APASSSTKKTQLQLEHLLLDLQMILNGINNYKNPK |
| | (T3A, R38E, F42A, K76E, | LTEMLTAKFYMPKKATELKHLQCLEEELKPLEEVL |
| | C125S) | NLAQSENFHLRPRDLISNINVIVLELKGSETTFMC |
| | | EYADETATIVEFLNRWITFSQSIISTLT |
| 201 | hIL-2 Mutein | APTSSSTKKTQLQLEHLLLDLQMILNGINNYKNPKL |
| | (R38E, F42K, K76E, C125S) | TEMLTKKFYMPKKATELKHLQCLEEELKPLEEVLN |
| | | LAQSENFHLRPRDLISNINVIVLELKGSETTFMCEY |
| | | ADETATIVEFLNRWITFSQSIISTLT |
| 202 | hIL-2 Mutein | APASSSTKKTQLQLEHLLLDLQMILNGINNYKNPK |
| | (T3A, R38E, F42K, K76E, | LTEMLTKKFYMPKKATELKHLQCLEEELKPLEEVL |
| | C125S) | NLAQSENFHLRPRDLISNINVIVLELKGSETTFMC |
| | | EYADETATIVEFLNRWITFSQSIISTLT |
| 203 | hIL-2 Mutein | APTSSSTKKTQLQLKHLLLDLQMILNGINNYKNPK |
| | (E15K, R38E, F42K, K76E, | LTEMLTKKFYMPKKATELKHLQCLEEELKPLEEVL |
| | C125S) | NLAQSENFHLRPRDLISNINVIVLELKGSETTFMC |
| | | EYADETATIVEFLNRWITFSQSIISTLT |
| 204 | hIL-2 Mutein | APASSSTKKTQLQLKHLLLDLQMILNGINNYKNPK |
| | (T3A, E15K, R38E, F42K, | LTEMLTKKFYMPKKATELKHLQCLEEELKPLEEVL |
| | K76E, C125S) | NLAQSENFHLRPRDLISNINVIVLELKGSETTFMC |
| | | EYADETATIVEFLNRWITFSQSIISTLT |
| 205 | hIL-2 Mutein | APTSSSTKKTQLQLEHLLLDLQMILNGINNYKNPKL |
| | (F42C, V69C, C125S) | TRMLTCKFYMPKKATELKHLQCLEEELKPLEECLN |
| | | LAQSKNFHLRPRDLISNINVIVLELKGSETTFMCEY |
| | | ADETATIVEFLNRWITFSQSIISTLT |
| 206 | hIL-2 Mutein | APASSSTKKTQLQLEHLLLDLQMILNGINNYKNPK |
| | (T3A, F42C, V69C, C125S) | LTRMLTCKFYMPKKATELKHLQCLEEELKPLEECL |
| | | NLAQSKNFHLRPRDLISNINVIVLELKGSETTFMC |
| | | EYADETATIVEFLNRWITFSQSIISTLT |
| 207 | hIL-2 Mutein | APTSSSTKKTQLQLEHLLLDLQMILNGINNYENPKL |
| | (K32E, F42C, V69C, C125S) | TRMLTCKFYMPKKATELKHLQCLEEELKPLEECLNL |
| | | AQSKNFHLRPRDLISNINVIVLELKGSETTFMCEY |
| | | ADETATIVEFLNRWITFSQSIISTLT |
| 208 | hIL-2 Mutein | APASSSTKKTQLQLEHLLLDLQMILNGINNYENPKL |
| | (T3A, K32E, F42C, V69C, | TRMLTCKFYMPKKATELKHLQCLEEELKPLEECLNL |
| | C125S) | AQSKNFHLRPRDLISNINVIVLELKGSETTFMCEY |
| | | ADETATIVEFLNRWITFSQSIISTLT |
| 209 | hIL-2 Mutein | APTSSSTKKTQLQLEHLLLDLQMILNGINNYKNPKL |
| | (F42C, V69C, K76E, C125S) | TRMLTCKFYMPKKATELKHLQCLEEELKPLEECLNL |
| | | AQSENFHLRPRDLISNINVIVLELKGSETTFMCEY |
| | | ADETATIVEFLNRWITFSQSIISTLT |
| 210 | hIL-2 Mutein | APASSSTKKTQLQLEHLLLDLQMILNGINNYKNPK |
| | (T3A, F42C, V69C, K76E, | LTRMLTCKFYMPKKATELKHLQCLEEELKPLEECL |
| | C125S) | NLAQSENFHLRPRDLISNINVIVLELKGSETTFMC |
| | | EYADETATIVEFLNRWITFSQSIISTLT |
| 211 | hIL-2 Mutein | APTSSSTKKTQLQLKHLLLDLQMILNGINNYKNPK |
| | (E15K, F42C, V69C, C125S) | LTRMLTCKFYMPKKATELKHLQCLEEELKPLEECL |
| | | NLAQSKNFHLRPRDLISNINVIVLELKGSETTFMC |
| | | EYADETATIVEFLNRWITFSQSIISTLT |
| 212 | hIL-2 Mutein | APASSSTKKTQLQLKHLLLDLQMILNGINNYKNPK |
| | (T3A, E15K, F42C, V69C, | LTRMLTCKFYMPKKATELKHLQCLEEELKPLEECL |
| | C125S) | NLAQSKNFHLRPRDLISNINVIVLELKGSETTFMC |
| | | EYADETATIVEFLNRWITFSQSIISTLT |
| 213 | hIL-2 Mutein | APTSSSTKKTQLQLKHLLLDLQMILNGINNYENPKL |
| | (E15K, K32E, F42C, V69C, | TRMLTCKFYMPKKATELKHLQCLEEELKPLEECLNL |
| | C125S) | AQSKNFHLRPRDLISNINVIVLELKGSETTFMCEY |
| | | ADETATIVEFLNRWITFSQSIISTLT |
| 214 | hIL-2 Mutein | APASSSTKKTQLQLKHLLLDLQMILNGINNYENPK |
| | (T3A, E15K, K32E, F42C, | LTRMLTCKFYMPKKATELKHLQCLEEELKPLEECL |
| | V69C, C125S) | NLAQSKNFHLRPRDLISNINVIVLELKGSETTFMC |
| | | EYADETATIVEFLNRWITFSQSIISTLT |
| 215 | hIL-2 Mutein | APTSSSTKKTQLQLKHLLLDLQMILNGINNYKNPK |
| | (E15K, F42C, V69C, K76E, | LTRMLTCKFYMPKKATELKHLQCLEEELKPLEECL |
| | C125S) | NLAQSENFHLRPRDLISNINVIVLELKGSETTFMC |
| | | EYADETATIVEFLNRWITFSQSIISTLT |
| 216 | hIL-2 Mutein | APASSSTKKTQLQLKHLLLDLQMILNGINNYKNPK |
| | (T3A, E15K, F42C, V69C, | LTRMLTCKFYMPKKATELKHLQCLEEELKPLEECL |
| | K76E, C125S) | NLAQSENFHLRPRDLISNINVIVLELKGSETTFMC |
| | | EYADETATIVEFLNRWITFSQSIISTLT |
| 217 | hIL-2 Fragment | NNYKNPKLTRML |
| | (N29 to L40) | |
| 218 | IL-15 Hinge Fragment- | QSMEIDAT |
| | containing Peptide 1 | |
| 219 | IL-15 Hinge Fragment- | QSMHIDAT |
| | containing peptide 2 | |
| 220 | IL-2-IL-15 Chimera 1 | APTSSSTKKTQLQLEHLLLDLQMILNGIQSMEIDAT |
| | (C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 221 | IL-2-IL-15 Chimera 2 | APASSSTKKTQLQLEHLLLDLQMILNGIQSMEIDAT |
| | (T3A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 222 | IL-2-IL-15 Chimera 3 | APTSSSTKKTQLQLEHLLLDLQMILNGIQSMHIDAT |
| | (E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 223 | IL-2-IL-15 Chimera 4 | APASSSTKKTQLQLEHLLLDLQMILNGIQSMHIDAT |
| | (T3A, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 224 | IL-2-IL-15 Chimera 5 | APTSSSTKKTQLQLKHLLLDLQMILNGIQSMEIDAT |
| | (E15K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 225 | IL-2-IL-15 Chimera 6 | APASSSTKKTQLQLKHLLLDLQMILNGIQSMEIDAT |
| | (T3A, E15K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 226 | IL-2-IL-15 Chimera 7 | APTSSSTKKTQLQLKHLLLDLQMILNGIQSMHIDAT |
| | (E15K, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 227 | IL-2-IL-15 Chimera 8 | APASSSTKKTQLQLKHLLLDLQMILNGIQSMHIDAT |
| | (T3A, E15K, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 228 | IL-2-IL-15 Chimera 9 | APTSSSTKKTQLQLKHLLLDLQMILNGIQSMHIDAT |
| | (E15K, E32H, S87D, C125S) | TFKFYMPKKATELKHLQCLEEELKPLEEVLNLAQS |
| | | KNFHLRPRDLIDNINVIVLELKGSETTFMCEYADET |
| | | ATIVEFLNRWITFSQSIISTLT |
| 229 | IL-2-IL-15 Chimera 10 | APASSSTKKTQLQLKHLLLDLQMILNGIQSMHIDAT |
| | (T3A, E15K, E32H, S87D, | TFKFYMPKKATELKHLQCLEEELKPLEEVLNLAQS |
| | C125S) | KNFHLRPRDLIDNINVIVLELKGSETTFMCEYADET |
| | | ATIVEFLNRWITFSQSIISTLT |
| 230 | IL-2-IL-15 Chimera 11 | APTSSSTKKTQLQLEELLLDLQMILNGIQSMEIDAT |
| | (H16E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 231 | IL-2-IL-15 Chimera 12 | APASSSTKKTQLQLEELLLDLQMILNGIQSMEIDAT |
| | (T3A, H16E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 232 | IL-2-IL-15 Chimera 13 | APTSSSTKKTQLQLEFLLLDLQMILNGIQSMEIDAT |
| | (H16F, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 233 | IL-2-IL-15 Chimera 14 | APASSSTKKTQLQLEFLLLDLQMILNGIQSMEIDAT |
| | (T3A, H16F, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 234 | IL-2-IL-15 Chimera 15 | APTSSSTKKTQLQLEILLLDLQMILNGIQSMEIDATT |
| | (H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 235 | IL-2-IL-15 Chimera 16 | APASSSTKKTQLQLEILLLDLQMILNGIQSMEIDATT |
| | (T3A, H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 236 | IL-2-IL-15 Chimera 17 | APTSSSTKKTQLQLEVLLLDLQMILNGIQSMEIDAT |
| | (H16V, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 237 | IL-2-IL-15 Chimera 18 | APASSSTKKTQLQLEVLLLDLQMILNGIQSMEIDAT |
| | (T3A, H16V, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 238 | IL-2-IL-15 Chimera 19 | APTSSSTKKTQLQLEHLLLTLQMILNGIQSMEIDAT |
| | (D20T, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 239 | IL-2-IL-15 Chimera 20 | APASSSTKKTQLQLEHLLLTLQMILNGIQSMEIDAT |
| | (T3A, D20T, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 240 | IL-2-IL-15 Chimera 21 | APTSSSTKKTQLQLEHLLLALQMILNGIQSMEIDAT |
| | (D20A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 241 | IL-2-IL-15 Chimera 22 | APASSSTKKTQLQLEHLLLALQMILNGIQSMEIDAT |
| | (T3A, D20A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 242 | IL-2-IL-15 Chimera 23 | APTSSSTKKTQLQLEHLLLELQMILNGIQSMEIDAT |
| | (D20E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 243 | IL-2-IL-15 Chimera 24 | APASSSTKKTQLQLEHLLLELQMILNGIQSMEIDAT |
| | (T3A, D20E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 244 | IL-2-IL-15 Chimera 25 | APTSSSTKKTQLQLEHLLLKLQMILNGIQSMEIDAT |
| | (D20K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 245 | IL-2-IL-15 Chimera 26 | APASSSTKKTQLQLEHLLLKLQMILNGIQSMEIDAT |
| | (T3A, D20K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 246 | IL-2-IL-15 Chimera 27 | APTSSSTKKTQLQLEHLLLDLQMILNGIQSMEIDAT |
| | (N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 247 | IL-2-IL-15 Chimera 28 | APASSSTKKTQLQLEHLLLDLQMILNGIQSMEIDAT |
| | (T3A, N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 248 | IL-2-IL-15 Chimera 29 | APTSSSTKKTQLQLEHLLLDLQMILNGIQSMEIDAT |
| | (192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 249 | IL-2-IL-15 Chimera 30 | APASSSTKKTQLQLEHLLLDLQMILNGIQSMEIDAT |
| | (T3A, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 250 | IL-2-IL-15 Chimera 31 | APTSSSTKKTQLQLEHLLLDLQMILNGIQSMEIDAT |
| | (192G, C125S) | TFKFYMPKKATELKHLQCLEEELKPLEEVLNLAQS |
| | | KNFHLRPRDLISNINVGVLELKGSETTFMCEYADET |
| | | ATIVEFLNRWITFSQSIISTLT |
| 251 | IL-2-IL-15 Chimera 32 | APASSSTKKTQLQLEHLLLDLQMILNGIQSMEIDAT |
| | (T3A, I92G, C125S) | TFKFYMPKKATELKHLQCLEEELKPLEEVLNLAQS |
| | | KNFHLRPRDLISNINVGVLELKGSETTFMCEYADET |
| | | ATIVEFLNRWITFSQSIISTLT |
| 252 | IL-2-IL-15 Chimera 33 | APTSSSTKKTQLQLEHLLLDLQMILNGIQSMEIDAT |
| | (I92D, C125S) | TFKFYMPKKATELKHLQCLEEELKPLEEVLNLAQS |
| | | KNFHLRPRDLISNINVDVLELKGSETTFMCEYADET |
| | | ATIVEFLNRWITFSQSIISTLT |
| 253 | IL-2-IL-15 Chimera 34 | APASSSTKKTQLQLEHLLLDLQMILNGIQSMEIDAT |
| | (T3A, I92D, C125S) | TFKFYMPKKATELKHLQCLEEELKPLEEVLNLAQS |
| | | KNFHLRPRDLISNINVDVLELKGSETTFMCEYADET |
| | | ATIVEFLNRWITFSQSIISTLT |
| 254 | IL-2-IL-15 Chimera 35 | APTSSSTKKTQLQLEHLLLDLQMILNGIQSMEIDAT |
| | (192E, C125S) | TFKFYMPKKATELKHLQCLEEELKPLEEVLNLAQS |
| | | KNFHLRPRDLISNINVEVLELKGSETTFMCEYADET |
| | | ATIVEFLNRWITFSQSIISTLT |
| 255 | IL-2-IL-15 Chimera 36 | APASSSTKKTQLQLEHLLLDLQMILNGIQSMEIDAT |
| | (T3A, I92E, C125S) | TFKFYMPKKATELKHLQCLEEELKPLEEVLNLAQS |
| | | KNFHLRPRDLISNINVEVLELKGSETTFMCEYADET |
| | | ATIVEFLNRWITFSQSIISTLT |
| 256 | IL-2-IL-15 Chimera 37 | APTSSSTKKTQLQLEELLLDLQMILNGIQSMEIDAT |
| | (H16E, I92A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 257 | IL-2-IL-15 Chimera 38 | APASSSTKKTQLQLEELLLDLQMILNGIQSMEIDAT |
| | (T3A, H16E, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 258 | IL-2-IL-15 Chimera 39 | APTSSSTKKTQLQLKHLLLDLQMILNGIQSMEIDAT |
| | (E15K, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 259 | IL-2-IL-15 Chimera 40 | APASSSTKKTQLQLKHLLLDLQMILNGIQSMEIDAT |
| | (T3A, E15K, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 260 | IL-2-IL-15 Chimera 41 | APTSSSTKKTQLQLKILLLDLQMILNGIQSMEIDATT |
| | (E15K, H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 261 | IL-2-IL-15 Chimera 42 | APTSSSTKKTQLQLKHLLLDLQMILNGIQSMEIDAT |
| | (E15K, N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 262 | IL-2-IL-15 Chimera 43 | APTSSSTKKTQLQLEHLCLDLQMILNGIQSMEIDAT |
| | (L18C) | TFKFYMPKKATELKHLQCLEEELKPLEEVLNLAQS |
| | | KNFHLRPRDLISNINVIVLELKGSETTFMCEYADET |
| | | ATIVEFLNRWITFCQSIISTLT |
| 263 | IL-2-IL-15 Chimera 44 | APASSSTKKTQLQLEHLCLDLQMILNGIQSMEIDAT |
| | (T3A, L18C) | TFKFYMPKKATELKHLQCLEEELKPLEEVLNLAQS |
| | | KNFHLRPRDLISNINVIVLELKGSETTFMCEYADET |
| | | ATIVEFLNRWITFCQSIISTLT |
| 264 | IL-2-IL-15 Chimera 45 | APTSSSTKKTQLQLEHLLLDLQMILNGIQSMEIDAT |
| | (C125S, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFSESIISTLT |
| 265 | IL-2-IL-15 Chimera 46 | APASSSTKKTQLQLEHLLLDLQMILNGIQSMEIDAT |
| | (T3A, C125S, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFSESIISTLT |
| 266 | IL-2-IL-15 Chimera 47 | APTSSSTKKTQLQLEHLLLDLQMILNGIQSMEIDAT |
| | (C125S, Q126K) | TFKFYMPKKATELKHLQCLEEELKPLEEVLNLAQS |
| | | KNFHLRPRDLISNINVIVLELKGSETTFMCEYADET |
| | | ATIVEFLNRWITFSKSIISTLT |
| 267 | IL-2-IL-15 Chimera 48 | APASSSTKKTQLQLEHLLLDLQMILNGIQSMEIDAT |
| | (T3A, C125S, Q126K) | TFKFYMPKKATELKHLQCLEEELKPLEEVLNLAQS |
| | | KNFHLRPRDLISNINVIVLELKGSETTFMCEYADET |
| | | ATIVEFLNRWITFSKSIISTLT |
| 268 | IL-2-IL-15 Chimera 49 | APTSSSTKKTQLQLEHLLLDLQMILNGIQSMEIDAT |
| | (C125S, Q126R) | TFKFYMPKKATELKHLQCLEEELKPLEEVLNLAQS |
| | | KNFHLRPRDLISNINVIVLELKGSETTFMCEYADET |
| | | ATIVEFLNRWITFSRSIISTLT |
| 269 | IL-2-IL-15 Chimera 50 | APASSSTKKTQLQLEHLLLDLQMILNGIQSMEIDAT |
| | (T3A, C125S, Q126R) | TFKFYMPKKATELKHLQCLEEELKPLEEVLNLAQS |
| | | KNFHLRPRDLISNINVIVLELKGSETTFMCEYADET |
| | | ATIVEFLNRWITFSRSIISTLT |
| 270 | IL-2-IL-15 Chimera 51 | APTSSSTKKTQLQLEHLLLDLQMILNGIQSMEIDAT |
| | (C125S, Q126S) | TFKFYMPKKATELKHLQCLEEELKPLEEVLNLAQS |
| | | KNFHLRPRDLISNINVIVLELKGSETTFMCEYADET |
| | | ATIVEFLNRWITFSSSIISTLT |
| 271 | IL-2-IL-15 Chimera 52 | APASSSTKKTQLQLEHLLLDLQMILNGIQSMEIDAT |
| | (T3A, C125S, Q126S) | TFKFYMPKKATELKHLQCLEEELKPLEEVLNLAQS |
| | | KNFHLRPRDLISNINVIVLELKGSETTFMCEYADET |
| | | ATIVEFLNRWITFSSSIISTLT |
| 272 | IL-2-IL-15 Chimera 53 | APTSSSTKKTQLQLEHLLLDLQMILNGIQSMEIDAT |
| | (C125S, Q126T) | TFKFYMPKKATELKHLQCLEEELKPLEEVLNLAQS |
| | | KNFHLRPRDLISNINVIVLELKGSETTFMCEYADET |
| | | ATIVEFLNRWITFSTSIISTLT |
| 273 | IL-2-IL-15 Chimera 54 | APASSSTKKTQLQLEHLLLDLQMILNGIQSMEIDAT |
| | (T3A, C125S, Q126T) | TFKFYMPKKATELKHLQCLEEELKPLEEVLNLAQS |
| | | KNFHLRPRDLISNINVIVLELKGSETTFMCEYADET |
| | | ATIVEFLNRWITFSTSIISTLT |
| 274 | IL-2-IL-15 Chimera 55 | APTSSSTKKTQLQLEHLLLDLQMILNGIQSMEIDAT |
| | (C125S, S130R) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIIRTLT |
| 275 | IL-2-IL-15 Chimera 56 | APASSSTKKTQLQLEHLLLDLQMILNGIQSMEIDAT |
| | (T3A, C125S, S130R) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIIRTLT |
| 276 | IL-2-IL-15 Chimera 57 | APTSSSTKKTQLQLEHLCLDLQMILNGIQSMEIDAT |
| | (L18C, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFCESIISTLT |
| 277 | IL-2-IL-15 Chimera 58 | APASSSTKKTQLQLEHLCLDLQMILNGIQSMEIDAT |
| | (T3A, L18C, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFCESIISTLT |
| 278 | IL-2-IL-15 Chimera 59 | APTSSSTKKTQLQLEHLCLDLQMILNGIQSMEIDAT |
| | (L18C, Q126K) | TFKFYMPKKATELKHLQCLEEELKPLEEVLNLAQS |
| | | KNFHLRPRDLISNINVIVLELKGSETTFMCEYADET |
| | | ATIVEFLNRWITFCKSIISTLT |
| 279 | IL-2-IL-15 Chimera 60 | APASSSTKKTQLQLEHLCLDLQMILNGIQSMEIDAT |
| | (T3A, L18C, Q126K) | TFKFYMPKKATELKHLQCLEEELKPLEEVLNLAQS |
| | | KNFHLRPRDLISNINVIVLELKGSETTFMCEYADET |
| | | ATIVEFLNRWITFCKSIISTLT |
| 280 | IL-2-IL-15 Chimera 61 | APTSSSTKKTQLQLEHLCLDLQMILNGIQSMEIDAT |
| | (L18C, Q126R) | TFKFYMPKKATELKHLQCLEEELKPLEEVLNLAQS |
| | | KNFHLRPRDLISNINVIVLELKGSETTFMCEYADET |
| | | ATIVEFLNRWITFCRSIISTLT |
| 281 | IL-2-IL-15 Chimera 62 | APASSSTKKTQLQLEHLCLDLQMILNGIQSMEIDAT |
| | (T3A, L18C, Q126R) | TFKFYMPKKATELKHLQCLEEELKPLEEVLNLAQS |
| | | KNFHLRPRDLISNINVIVLELKGSETTFMCEYADET |
| | | ATIVEFLNRWITFCRSIISTLT |
| 282 | IL-2-IL-15 Chimera 63 | APTSSSTKKTQLQLEHLCLDLQMILNGIQSMEIDAT |
| | (L18C, Q126S) | TFKFYMPKKATELKHLQCLEEELKPLEEVLNLAQS |
| | | KNFHLRPRDLISNINVIVLELKGSETTFMCEYADET |
| | | ATIVEFLNRWITFCSSIISTLT |
| 283 | IL-2-IL-15 Chimera 64 | APASSSTKKTQLQLEHLCLDLQMILNGIQSMEIDAT |
| | (T3A, L18C, Q126S) | TFKFYMPKKATELKHLQCLEEELKPLEEVLNLAQS |
| | | KNFHLRPRDLISNINVIVLELKGSETTFMCEYADET |
| | | ATIVEFLNRWITFCSSIISTLT |
| 284 | IL-2-IL-15 Chimera 65 | APTSSSTKKTQLQLEHLLLDLQMILNGIQSMEIDAT |
| | (192A, C125S, Q126E) | TFKFYMPKKATELKHLQCLEEELKPLEEVLNLAQS |
| | | KNFHLRPRDLISNINVAVLELKGSETTFMCEYADET |
| | | ATIVEFLNRWITFSESIISTLT |
| 285 | IL-2-IL-15 Chimera 66 | APASSSTKKTQLQLEHLLLDLQMILNGIQSMEIDAT |
| | (T3A, I92A, C125S, Q126E) | TFKFYMPKKATELKHLQCLEEELKPLEEVLNLAQS |
| | | KNFHLRPRDLISNINVAVLELKGSETTFMCEYADET |
| | | ATIVEFLNRWITFSESIISTLT |
| 286 | IL-2-IL-15 Chimera 67 | APTSSSTKKTQLQLEHLLLDLQMILNGIQSMEIDAT |
| | (C125S, Q126T, S130R) | TFKFYMPKKATELKHLQCLEEELKPLEEVLNLAQS |
| | | KNFHLRPRDLISNINVIVLELKGSETTFMCEYADET |
| | | ATIVEFLNRWITFSTSIIRTLT |
| 287 | IL-2-IL-15 Chimera 68 | APASSSTKKTQLQLEHLLLDLQMILNGIQSMEIDAT |
| | (T3A, C125S, Q126T, | TFKFYMPKKATELKHLQCLEEELKPLEEVLNLAQS |
| | S130R) | KNFHLRPRDLISNINVIVLELKGSETTFMCEYADET |
| | | ATIVEFLNRWITFSTSIIRTLT |
| 288 | Mouse IL-2-IL-15 Chimera | APTSSSTSSSTAEAQQQQQQQQQQQQHLEQLLMDL |
| | | QELLSRMQSMEIDATTFKFYLPKQATELKDLQCLE |
| | | DELGPLRHVLDLTQSKSFQLEDAENFISNIRVTVVK |
| | | LKGSDNTFECQFDDESATVVDFLRRWIAFSQSIISTS |
| | | PQ |
| 289 | hIL-2Rα | ELCDDDPPEIPHATFKAMAYKEGTMLNCECKRGFR |
| | | RIKSGSLYMLCTGNSSHSSWDNQCQCTSSATRNTT |
| | | KQVTPQPEEQKERKTTEMQSPMQPVDQASLPGHC |
| | | REPPPWENEATERIYHFVVGQMVYYQCVQGYRAL |
| | | HRGPAESVCKMTHGKTRWTQPQLICTGEMETSQFP |
| | | GEEKPQASPEGRPESETSCLVTTTDFQIQTEMAATM |
| | | ETSIFTTEYQ |
| 290 | hIL-2Rβα | AVNGTSQFTCFYNSRANISCVWSQDGALQDTSCQV |
| | | HAWPDRRRWNQTCELLPVSQASWACNLILGAPDS |
| | | QKLTTVDIVTLRVLCREGVRWRVMAIQDFKPFENL |
| | | RLMAPISLQVVHVETHRCNISWEISQASHYFERHLE |
| | | FEARTLSPGHTWEEAPLLTLKQKQEWICLETLTPDT |
| | | QYEFQVRVKPLQGEFTTWSPWSQPLAFRTKPAALG |
| | | KD |
| 291 | hIL-2Rγ | LNTTILTPNGNEDTTADFFLTTMPTDSLSVSTLPLPE |
| | | VQCFVFNVEYMNCTWNSSSEPQPTNLTLHYWYKN |
| | | SDNDKVQKCSHYLFSEEITSGCQLQKKEIHLYQTFV |
| | | VQLQDPREPRRQATQMLKLQNLVIPWAPENLTLH |
| | | KLSESQLELNWNNRFLNHCLEHLVQYRTDWDHSW |
| | | TEQSVDYRHKFSLPSVDGQKRYTFRVRSRFNPLCG |
| | | SAQHWSEWSHPIHWGSNTSKENPFLFALEA |
| 292 | Anti-PD-1-A- IL-2-IL-15 | QVQLVESGGGVVQPGRSLRLDCKASGITFSNSGMH |
| | Chimera 2 (T3A, C125S) | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPASSSTKKTQLQLEHLLLDLQMILNGIQSM |
| | | EIDATTFKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 293 | Anti-PD-1- A- IL-2-IL-15 | QVQLVESGGGVVQPGRSLRLDCKASGITFSNSGMH |
| | Chimera 44 (T3A, L18C) | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPCQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPASSSTKKTQLQLEHLCLDLQMILNGIQSM |
| | | EIDATTFKFYMPKKATELKHLQCLEEELKPLEEVLN |
| | | LAQSKNFHLRPRDLISNINVIVLELKGSETTFMCEY |
| | | ADETATIVEFLNRWITFCQSIISTLT |
| 29 | Anti-PD-1- A-IL-2 Chimera | QVQLVESGGGVVQPGRSLRLDCKASGITFSNSGMH |
| | 54 (T3A, C125S, Q126T) | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPASSSTKKTQLQLEHLLLDLQMILNGIQSM |
| | | EIDATTFKFYMPKKATELKHLQCLEEELKPLEEVLN |
| | | LAQSKNFHLRPRDLISNINVIVLELKGSETTFMCEY |
| | | ADETATIVEFLNRWITFSTSIISTLT |
| 295 | Anti-PD-1-A- IL-2-IL-15 | QVQLVESGGGVVQPGRSLRLDCKASGITFSNSGMH |
| | Chimera 56 (T3A, C125S, | WVRQAPGKGLEWVAVIWYDGSKRYYADSVKGRF |
| | S130R) | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPASSSTKKTQLQLEHLLLDLQMILNGIQSM |
| | | EIDATTFKFYMPKKATELKHLQCLEEELKPLEEVLN |
| | | LAQSKNFHLRPRDLISNINVIVLELKGSETTFMCEY |
| | | ADETATIVEFLNRWITFSQSIIRTLT |
| 296 | Anti-PD-1- A- IL-2-IL-15 | QVQLVESGGGVVQPGRSLRLDCKASGITFSNSGMH |
| | Chimera 58 (T3A, L18C, | WVRQAPGKGLEWVAVIWYDGSKRYYADSVKGRF |
| | Q126E) | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPCQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPASSSTKKTQLQLEHLCLDLQMILNGIQSM |
| | | EIDATTFKFYMPKKATELKHLQCLEEELKPLEEVLN |
| | | LAQSKNFHLRPRDLISNINVIVLELKGSETTFMCEY |
| | | ADETATIVEFLNRWITFCESIISTLT |
| 297 | Anti-PD-1-A- IL-2-IL-15 | QVQLVESGGGVVQPGRSLRLDCKASGITFSNSGMH |
| | Chimera 64 (T3A, L18C, | WVRQAPGKGLEWVAVIWYDGSKRYYADSVKGRF |
| | Q126S) | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPCQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPASSSTKKTQLQLEHLCLDLQMILNGIQSM |
| | | EIDATTFKFYMPKKATELKHLQCLEEELKPLEEVLN |
| | | LAQSKNFHLRPRDLISNINVIVLELKGSETTFMCEY |
| | | ADETATIVEFLNRWITFCSSIISTLT |
| 298 | Anti-PD-1- A- IL-2-IL-15 | QVQLVESGGGVVQPGRSLRLDCKASGITFSNSGMH |
| | Chimera 68 (T3A, C125S, | WVRQAPGKGLEWVAVIWYDGSKRYYADSVKGRF |
| | Q126T, S130R) | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPASSSTKKTQLQLEHLLLDLQMILNGIQSM |
| | | EIDATTFKFYMPKKATELKHLQCLEEELKPLEEVLN |
| | | LAQSKNFHLRPRDLISNINVIVLELKGSETTFMCEY |
| | | ADETATIVEFLNRWITFSTSIIRTLT |
| 299 | Anti-PD-1- A-IL-2 (T3A, | QVQLVESGGGVVQPGRSLRLDCKASGITFSNSGMH |
| | F42A, Y45A, L72G, C125S) | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPASSSTKKTQLQLEHLLLDLQMILNGINNY |
| | | KNPKLTRMLTAKFAMPKKATELKHLQCLEEELKPL |
| | | EEVLNGAQSKNFHLRPRDLISNINVIVLELKGSETTF |
| | | MCEYADETATIVEFLNRWITFAQSIISTLT |
| 300 | Anti-PD-1-A (hole) | QVQLVESGGGVVQPGRSLRLDCKASGITFSNSGMH |
| | | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLSCAVKGFYPSDIAVEWESNGQPENN |
| | | YKTTPPVLDSDGSFFLVSRLTVDKSRWQEGNVFSC |
| | | SVMHEALHNHYTQKSLSLSLGK |
| 301 | IL-2-IL-15 Chimera 69 | APASSSTKKTQLQLEHLSLDLQMILNGIQSMEIDAT |
| | (T3A, L18S, C125S, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFSESIISTLT |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A fusion protein comprising an interleukin-2 (IL-2) domain and an anti-PD-1 antibody; wherein the IL-2 domain comprises: (i) an amino acid substitution at position L18, Q126, or S130; and (ii) a replacement of the amino acid residues from positions N29 to L40 with an IL-15 hinge fragment-containing peptide; a disulfide bond formed between two amino acid residues from positions N30 to L80; or an amino acid substitution at position E15, H16, D20, K32, R38, M39, L40, T41, F42, K43, F44, Y45, K76, S87, N88, or 192.

2. An interleukin-2 (IL-2) mutein comprising: (i) an amino acid substitution at position L18, Q126, or S130; and (ii) a replacement of the amino acid residues from positions N29 to L40 with an IL-15 hinge fragment-containing peptide; a disulfide bond formed between two amino acid residues from positions N30 to L80; or an amino acid substitution at position E15, H16, D20, K32, R38, M39, L40, T41, F42, K43, F44, Y45, K76, S87, N88, or 192.

3. The fusion protein of claim 1 or the IL-2 mutein of claim 2, wherein the IL-2 domain or IL-2 mutein comprises an amino acid substitution at position L18.

4. The fusion protein or IL-2 mutein of any one of claim 1 to 3, wherein the IL-2 domain or IL-2 mutein comprises an amino acid substitution of L18C.

5. The fusion protein or IL-2 mutein of any one of claim 1 to 3, wherein the IL-2 domain or IL-2 mutein comprises an amino acid substitution of L18S.

6. The fusion protein or IL-2 mutein of any one of claim 1 to 5, wherein the IL-2 domain or IL-2 mutein comprises an amino acid substitution at position Q126.

7. The fusion protein or IL-2 mutein of any one of claim 1 to 6, wherein the IL-2 domain or IL-2 mutein comprises an amino acid substitution of Q126E, Q126K, Q126R, Q126S, or Q126T.

8. The fusion protein or IL-2 mutein of any one of claim 1 to 7, wherein the IL-2 domain or IL-2 mutein comprises an amino acid substitution of Q126E, Q126K, or Q126R.

9. The fusion protein or IL-2 mutein of any one of claim 1 to 8, wherein the IL-2 domain or IL-2 mutein comprises an amino acid substitution of Q126E.

10. The fusion protein or IL-2 mutein of any one of claim 1 to 9, wherein the IL-2 domain or IL-2 mutein comprises an amino acid substitution at position S130.

11. The fusion protein or IL-2 mutein of any one of claim 1 to 10, wherein the IL-2 domain or IL-2 mutein comprises an amino acid substitution of S130A, S130E, S130Q, or S130R.

12. The fusion protein or IL-2 mutein of any one of claim 1 to 10, wherein the IL-2 domain or IL-2 mutein comprises amino acid substitution of (i) Q126E or Q126T; (ii) L18C, and Q126E, Q126K, Q126R, or Q126S; (iii) L18S and Q126E; (iv) Q126T and S130R; or (v) L18C, Q126E, and S130E.

13. The fusion protein or IL-2 mutein of any one of claim 1 to 11, wherein the IL-2 domain or IL-2 mutein comprises amino acid substitutions of L18S and Q126E.

14. The fusion protein or IL-2 mutein of any one of claim 1 to 13, wherein the IL-2 domain or IL-2 mutein comprises a replacement of the amino acid residues from positions N29 to L40 with an IL-15 hinge fragment-containing peptide, and optionally an amino acid substitution at position E15, H16, D20, S87, N88, or 192.

15. The fusion protein or IL-2 mutein of any one of claim 1 to 14, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219, and optionally an amino acid substitution of E15K, H16E, H16I, H16V, D20A, D20E, D20K, D20T, S87D, N88A, I92A, I92D, I92E, or I92G.

16. The fusion protein or IL-2 mutein of any one of claim 1 to 15, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219.

17. The fusion protein or IL-2 mutein of any one of claim 1 to 16, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219, and optionally an amino acid substitution of E15K.

18. The fusion protein or IL-2 mutein of any one of claim 1 to 17, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219, and optionally an amino acid substitution of H16E, H16F, H16I, or H16V.

19. The fusion protein or IL-2 mutein of any one of claim 1 to 18, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219, and optionally an amino acid substitution of D20A, D20E, D20K, or D20T.

20. The fusion protein or IL-2 mutein of any one of claim 1 to 19, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219, and optionally an amino acid substitution of S87D.

21. The fusion protein or IL-2 mutein of any one of claim 1 to 20, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219, and optionally an amino acid substitution of N88A.

22. The fusion protein or IL-2 mutein of any one of claim 1 to 21, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219, and optionally an amino acid substitution of I92A, I92D, I92E, or I92G.

23. The fusion protein or IL-2 mutein of any one of claim 1 to 22, wherein the IL-2 domain or IL-2 mutein comprises a replacement of the amino acid residues from positions N29 to L40 having an amino acid sequence of SEQ ID NO: 217 with an IL-15 hinge fragment-containing peptide having an amino acid sequence of SEQ ID NO: 218 or 219, and one or two amino acid substitutions of E15K, H16I, D20T, N88A, or I92A.

24. The fusion protein or IL-2 mutein of any one of claim 1 to 23, wherein the IL-2 domain or IL-2 mutein comprises an amino acid substitution at position E15, H16, D20, K32, R38, L40, F42, K76, S87, N88, or 192.

25. The fusion protein or IL-2 mutein of any one of claim 1 to 24, wherein the IL-2 domain or IL-2 mutein comprises an amino acid substitution at position E15, D20, H16, N88, or 192.

26. The fusion protein or IL-2 mutein of any one of claim 1 to 25, wherein the IL-2 domain or IL-2 mutein comprises an amino acid substitution at position E15.

27. The fusion protein or IL-2 mutein of any one of claim 1 to 26, wherein the IL-2 domain or IL-2 mutein comprises an amino acid substitution of E15K.

28. The fusion protein or IL-2 mutein of any one of claim 1 to 27, wherein the IL-2 domain or IL-2 mutein comprises an amino acid substitution at position H16.

29. The fusion protein or IL-2 mutein of any one of claim 1 to 28, wherein the IL-2 domain or IL-2 mutein comprises an amino acid substitution of H16E, H16I, or H16V.

30. The fusion protein or IL-2 mutein of any one of claim 1 to 29, wherein the IL-2 domain or IL-2 mutein comprises an amino acid substitution at position D20.

31. The fusion protein or IL-2 mutein of any one of claim 1 to 30, wherein the IL-2 domain or IL-2 mutein comprises an amino acid substitution of D20A, D20E, D20K, or D20T.

32. The fusion protein or IL-2 mutein of any one of claim 1 to 31, wherein the IL-2 domain or IL-2 mutein comprises an amino acid substitution at position K32.

33. The fusion protein or IL-2 mutein of any one of claim 1 to 32, wherein the IL-2 domain or IL-2 mutein comprises an amino acid substitution of K32E.

34. The fusion protein or IL-2 mutein of any one of claim 1 to 33, wherein the IL-2 domain or IL-2 mutein comprises an amino acid substitution at position R38.

35. The fusion protein or IL-2 mutein of any one of claim 1 to 34, wherein the IL-2 domain or IL-2 mutein comprises an amino acid substitution of R38E or R38N.

36. The fusion protein or IL-2 mutein of any one of claim 1 to 35, wherein the IL-2 domain or IL-2 mutein comprises an amino acid substitution at position L40.

37. The fusion protein or IL-2 mutein of any one of claim 1 to 36, wherein the IL-2 domain or IL-2 mutein comprises an amino acid substitution of L40T.

38. The fusion protein or IL-2 mutein of any one of claim 1 to 37, wherein the IL-2 domain or IL-2 mutein comprises an amino acid substitution at position F42.

39. The fusion protein or IL-2 mutein of any one of claim 1 to 38, wherein the IL-2 domain or IL-2 mutein comprises s an amino acid substitution of F42A or F42K.

40. The fusion protein or IL-2 mutein of any one of claim 1 to 39, wherein the IL-2 domain or IL-2 mutein comprises an amino acid substitution at position K76.

41. The fusion protein or IL-2 mutein of any one of claim 1 to 40, wherein the IL-2 domain or IL-2 mutein comprises an amino acid substitution of K76E.

42. The fusion protein or IL-2 mutein of any one of claim 1 to 41, wherein the IL-2 domain or IL-2 mutein comprises an amino acid substitution at position S87.

43. The fusion protein or IL-2 mutein of any one of claim 1 to 42, wherein the IL-2 domain or IL-2 mutein comprises an amino acid substitution of S87D.

44. The fusion protein or IL-2 mutein of any one of claim 1 to 43, wherein the IL-2 domain or IL-2 mutein comprises an amino acid substitution at position N88.

45. The fusion protein or IL-2 mutein of any one of claim 1 to 44, wherein the IL-2 domain or IL-2 mutein comprises an amino acid substitution of N88A.

46. The fusion protein or IL-2 mutein of any one of claim 1 to 45, wherein the IL-2 domain or IL-2 mutein comprises an amino acid substitution at position 192.

47. The fusion protein or IL-2 mutein of any one of claim 1 to 46, wherein the IL-2 domain or IL-2 mutein comprises an amino acid substitution of I92A, I92D, I92E, or I92G.

48. The fusion protein or IL-2 mutein of any one of claim 1 to 47, wherein the IL-2 domain or IL-2 mutein comprises an amino acid substitution of I92A.

49. The fusion protein or IL-2 mutein of any one of claim 1 to 48, wherein the IL-2 domain or IL-2 mutein comprises amino acid substitution of I92A and Q126E.

50. The fusion protein or IL-2 mutein of any one of claim 1 to 49, wherein the IL-2 domain or IL-2 mutein comprises amino acid substitutions at positions R38 and L40.

51. The fusion protein or IL-2 mutein of any one of claim 1 to 50, wherein the IL-2 domain or IL-2 mutein comprises amino acid substitutions of R38N and L40T.

52. The fusion protein or IL-2 mutein of any one of claim 1 to 51, wherein the IL-2 domain or IL-2 mutein comprises amino acid substitutions at positions E15, R38, and L40.

53. The fusion protein or IL-2 mutein of any one of claim 1 to 52, wherein the IL-2 domain or IL-2 mutein comprises amino acid substitutions of E15K, R38N, and L40T.

54. The fusion protein or IL-2 mutein of any one of claim 1 to 53, wherein the IL-2 domain or IL-2 mutein comprises amino acid substitutions at positions E15, K32, R38, and L40.

55. The fusion protein or IL-2 mutein of any one of claim 1 to 54, wherein the IL-2 domain or IL-2 mutein comprises amino acid substitutions of E15K, K32E, R38N, and L40T.

56. The fusion protein or IL-2 mutein of any one of claim 1 to 55, wherein the IL-2 domain or IL-2 mutein comprises amino acid substitutions at positions E15, R38, L40, and K76.

57. The fusion protein or IL-2 mutein of any one of claim 1 to 56, wherein the IL-2 domain or IL-2 mutein comprises amino acid substitutions of E15K, R38N, L40T, and K76E.

58. The fusion protein or IL-2 mutein of any one of claim 1 to 57, wherein the IL-2 domain or IL-2 mutein is N-glycosylated.

59. The fusion protein or IL-2 mutein of any one of claim 1 to 58, wherein the IL-2 domain or IL-2 mutein comprises amino acid substitutions at positions R38 and F42.

60. The fusion protein or IL-2 mutein of any one of claim 1 to 59, wherein the IL-2 domain or IL-2 mutein comprises amino acid substitutions of R38E and F42A or F42K.

61. The fusion protein or IL-2 mutein of any one of claim 1 to 60, wherein the IL-2 domain or IL-2 mutein comprises amino acid substitutions at positions K32, R38, and F42.

62. The fusion protein or IL-2 mutein of any one of claim 1 to 61, wherein the IL-2 domain or IL-2 mutein comprises amino acid substitutions of K32E, R38E, and F42A or F42K.

63. The fusion protein or IL-2 mutein of any one of claim 1 to 61, wherein the IL-2 domain or IL-2 mutein comprises amino acid substitutions at positions E15, K32, R38, and F42.

64. The fusion protein or IL-2 mutein of any one of claim 1 to 63, wherein the IL-2 domain or IL-2 mutein comprises amino acid substitutions of E15K, K32E, R38E, and F42A or F42K.

65. The fusion protein or IL-2 mutein of any one of claim 1 to 59, wherein the IL-2 domain or IL-2 mutein comprises amino acid substitutions at positions R38, F42, and K76.

66. The fusion protein or IL-2 mutein of any one of claim 1 to 59 and 65, wherein the IL-2 domain or IL-2 mutein comprises amino acid substitutions of R38E, F42A or F42K, and K76E.

67. The fusion protein or IL-2 mutein of any one of claim 1 to 59, wherein the IL-2 domain or IL-2 mutein comprises amino acid substitutions at positions E15, R38, F42, and K76.

68. The fusion protein or IL-2 mutein of any one of claim 1 to 59 and 67, wherein the IL-2 domain or IL-2 mutein comprises amino acid substitutions of E15K, R38E, F42A or F42K, and K76E.

69. The fusion protein or IL-2 mutein of any one of claim 1 to 68, wherein the IL-2 domain or IL-2 mutein comprises a disulfide bond formed between two different amino acid residues from positions N30 to L80.

70. The fusion protein or IL-2 mutein of any one of claim 1 to 69, wherein the IL-2 domain or IL-2 mutein comprises a disulfide bond formed between two different amino acid residues, each independently at K35, R38, F42, Y45, E62, V69, or L72.

71. The fusion protein or IL-2 mutein of any one of claim 1 to 70, wherein the IL-2 domain or IL-2 mutein comprises a disulfide bond formed between K35C and L72C, R38C and L72C, F42C and V69C, Y42C and L72C, or Y45C and E62C.

72. The fusion protein or IL-2 mutein of any one of claim 1 to 71, wherein the IL-2 domain or IL-2 mutein comprises a disulfide bond formed between F42C and V69C.

73. The fusion protein or IL-2 mutein of any one of claim 1 to 72, wherein the IL-2 domain or IL-2 mutein comprises a disulfide bond formed between F42C and V69C and an amino acid substitution of E15K, K32E, or K76E.

74. The fusion protein or IL-2 mutein of claim 73, wherein the IL-2 domain or IL-2 mutein comprises a disulfide bond formed between F42C and V69C and an amino acid substitution of E15K.

75. The fusion protein or IL-2 mutein of claim 73, wherein the IL-2 domain or IL-2 mutein comprises a disulfide bond formed between F42C and V69C and an amino acid substitution of K32E.

76. The fusion protein or IL-2 mutein of claim 73, wherein the IL-2 domain or IL-2 mutein comprises a disulfide bond formed between F42C and V69C and an amino acid substitution of K76E.

77. The fusion protein or IL-2 mutein of claim 73, wherein the IL-2 domain or IL-2 mutein comprises a disulfide bond formed between F42C and V69C and amino acid substitutions of E15K and K32E.

78. The fusion protein or IL-2 mutein of claim 73, wherein the IL-2 domain or IL-2 mutein comprises a disulfide bond formed between F42C and V69C and amino acid substitutions of E15K and K76E.

79. The fusion protein of claim 1 or the IL-2 mutein of claim 2, wherein the IL-2 domain or IL-2 mutein comprises an amino acid sequence of any one of SEQ ID NOs: 185 to 261, wherein the IL-2 mutein comprises an amino acid substitution of L18C, L18S, Q126E, Q126K, Q126R, Q126S, Q126T, S130A, S130E, S130Q, or S130R; wherein the amino acid residue at position T3 is T or A, and the amino acid residue at position C125 is C or S; and
wherein, when the IL-2 mutein comprises an amino acid substitution of L18C, the amino acid residue at position C125 is C.

80. The fusion protein of claim 1 or the IL-2 mutein of claim 2, wherein the IL-2 domain or IL-2 mutein comprises an amino acid sequence of any one of SEQ ID NO: 262 to 287 and 301.

81. A fusion protein comprising an IL-2 domain and a half-life-extension domain, wherein the IL-2 domain comprises the amino acid sequence of the IL-2 mutein of any one of claims 2 to 80.

82. The fusion protein of claim 81, wherein the half-life-extension domain comprises an albumin binding domain, a fragment crystallizable (Fc) domain, a serum albumin, a polyethylene glycol group, or a fatty acyl group.

83. The fusion protein of claim 81 or 82, wherein the half-life-extension domain is an albumin binding domain.

84. The fusion protein of claim 82 or 83, wherein the albumin binding domain is an antibody or a fragment thereof that binds to a human serum albumin (HSA).

85. The fusion protein of claim 84, wherein the albumin binding domain is an anti-HSA single domain antibody.

86. The fusion protein of claim 84 or 85, wherein the albumin binding domain is an anti-HSA V_{H}H single domain antibody.

87. The fusion protein of any one of claims 81 to 86, comprising an IL-2 domain, an anti-HSA V_{H}H single domain antibody, and optionally a peptide linker; wherein the C-terminus of the anti-HSA V_{H}H single domain antibody is connected to the N-terminus of the IL-2 domain directly or via the peptide linker.

88. The fusion protein of any one of claims 85 to 87, wherein the single domain antibody comprises (i) a CDR1 of SEQ ID NO: 1, a CDR2 of SEQ ID NO: 2, and a CDR3 of SEQ ID NO: 3; or (ii) a CDR1 of SEQ ID NO: 9, a CDR2 of SEQ ID NO: 10, and a CDR3 of SEQ ID NO: 11.

89. The fusion protein of any one of claims 85 to 88, wherein the single domain antibody has an amino acid sequence of SEQ ID NO: 8 or 15.

90. The fusion protein of claim 81 or 82, wherein the half-life-extension domain is a Fc domain.

91. The fusion protein of claim 90, comprising an IL-2 domain, an Fc domain comprising first and second peptide chains, and optionally a peptide linker; wherein the C-terminus of the first peptide chain of the Fc domain is connected to the N-terminus of the IL-2 domain directly or via the peptide linker.

92. The fusion protein of any one of claims 81 to 91, further comprising a programmed cell death-1 (PD-1) binding domain.

93. The fusion protein of claim 92, wherein the PD-1 binding domain is an anti-PD-1 single domain antibody.

94. The fusion protein of claim 92 or 93, wherein the PD-1 binding domain is an anti-PD-1 V_{H}H single domain antibody.

95. The fusion protein of any one of claims 92 to 94, comprising an IL-2 domain, an anti-PD-1 V_{H}H single domain antibody, and an Fc domain.

96. The fusion protein of any one of claims 92 to 95, comprising an IL-2 domain, first and second PD-1 binding domains, an Fc domain comprising first and second peptide chains, and optionally a peptide linker; wherein a C-terminus of the first PD-1 binding domain is connected to the N-terminus of the first peptide chain of the Fc domain directly or via a peptide linker, and the C-terminus of the first peptide chain of the Fc domain is connected to the N-terminus of the IL-2 domain directly or via the peptide linker; and wherein a C-terminus of the second PD-1 binding domain is connected to the N-terminus of the second peptide chain of the Fc domain directly or via a peptide linker.

97. The fusion protein of any one of claims 94 to 96, wherein each anti-PD-1 V_{H}H single domain antibody independently comprises: (i) a CDR1 of SEQ ID NO: 119, a CDR2 of SEQ ID NO: 120, and a CDR3 of SEQ ID NO: 121; (ii) a CDR1 of SEQ ID NO: 123, a CDR2 of SEQ ID NO: 124, and a CDR3 of SEQ ID NO: 125; (iii) a CDR1 of SEQ ID NO: 127, a CDR2 of SEQ ID NO: 128, and a CDR3 of SEQ ID NO: 129; (iv) a CDR1 of SEQ ID NO: 131, a CDR2 of SEQ ID NO: 132, and a CDR3 of SEQ ID NO: 133; or (v) a CDR1 of SEQ ID NO: 135, a CDR2 of SEQ ID NO: 136, and a CDR3 of SEQ ID NO: 137.

98. The fusion protein of any one of claims 90 to 97, wherein the Fc domain is a hIgG1 Fc domain, a hIgG2 Fc domain, a hIgG4 Fc domain, or a mutein thereof.

99. The fusion protein of any one of claims 90 to 98, wherein the Fc domain is in a knobs-in-holes configuration.

100. The fusion protein of claim 92, wherein the anti-PD-1 binding domain and the FC domain form parts of an intact anti-PD-1 antibody comprising two light chains and two heavy chains.

101. The fusion protein of any one of claims 1 to 80 and 100, comprising an IL-2 domain, and an intact anti-PD-1 antibody comprising two light chains and two heavy chains.

102. The fusion protein of any one of claims 1 to 80, 100, and 101, comprising an IL-2 domain, an intact anti-PD-1 antibody comprising first and second light chains and first and second heavy chains, and optionally a peptide linker; wherein the C-terminus of the first heavy chain of the intact anti-PD-1 antibody is connected to the N-terminus of the IL-2 domain directly or via the peptide linker.

103. The fusion protein of any one of claims 1 to 80 and 100, comprising two IL-2 domains, and an intact anti-PD-1 antibody comprising two light chains and two heavy chains.

104. The fusion protein of any one of claims 1 to 80, 100, and 103, comprising first and second IL-2 domains, an intact anti-PD-1 antibody comprising first and second light chains and first and second heavy chains, and optionally first and second peptide linkers; wherein the C-terminus of the first heavy chain of the intact anti-PD-1 antibody is connected to the N-terminus of the first IL-2 domain directly or via the first peptide linker, and the C-terminus of the second heavy chain of the intact anti-PD-1 antibody is connected to the N-terminus of the second IL-2 domain directly or via the second peptide linker.

105. The fusion protein of any one of claims 1 to 80, and 100 to 104, wherein the PD-1 antibody comprises:
(i) a CDRL1 of SEQ ID NO: 29, a CDRL2 of DAS, a CDRL3 of SEQ ID NO: 30, a CDRH1 of SEQ ID NO: 31, a CDRH2 of SEQ ID NO: 32, and a CDRH3 of SEQ ID NO: 33;
(ii) a CDRL1 of SEQ ID NO: 38, a CDRL2 of LAS, a CDRL3 of SEQ ID NO: 39, a CDRH1 of SEQ ID NO: 40, a CDRH2 of SEQ ID NO: 41, and a CDRH3 of SEQ ID NO: 42;
(iii) a CDRL1 of SEQ ID NO: 47, a CDRL2 of KVS, a CDRL3 of SEQ ID NO: 48, a CDRH1 of SEQ ID NO: 49, a CDRH2 of SEQ ID NO: 50, and a CDRH3 of SEQ ID NO: 51;
(iv) a CDRL1 of SEQ ID NO: 56, a CDRL2 of TAT, a CDRL3 of SEQ ID NO: 57, a CDRH1 of SEQ ID NO: 58, a CDRH2 of SEQ ID NO: 59, and a CDRH3 of SEQ ID NO: 60;
(v) a CDRL1 of SEQ ID NO: 65, a CDRL2 of AAS, a CDRL3 of SEQ ID NO: 66, a CDRH1 of SEQ ID NO: 67, a CDRH2 of SEQ ID NO: 68, and a CDRH3 of SEQ ID NO: 69;
(vi) a CDRL1 of SEQ ID NO: 74, a CDRL2 of WAS, a CDRL3 of SEQ ID NO: 75, a CDRH1 of SEQ ID NO: 76, a CDRH2 of SEQ ID NO: 77, and a CDRH3 of SEQ ID NO: 78;
(vii) a CDRL1 of SEQ ID NO: 83, a CDRL2 of YAF, a CDRL3 of SEQ ID NO: 84, a CDRH1 of SEQ ID NO: 85, a CDRH2 of SEQ ID NO: 86, and a CDRH3 of SEQ ID NO: 87;
(viii) a CDRL1 of SEQ ID NO: 92, a CDRL2 of AAS, a CDRL3 of SEQ ID NO: 93, a CDRH1 of SEQ ID NO: 94, a CDRH2 of SEQ ID NO: 95, and a CDRH3 of SEQ ID NO: 96;
(ix) a CDRL1 of SEQ ID NO: 101, a CDRL2 of WAS, a CDRL3 of SEQ ID NO: 102, a CDRH1 of SEQ ID NO: 103, a CDRH2 of SEQ ID NO: 104, and a CDRH3 of SEQ ID NO: 105; or
(x) a CDRL1 of SEQ ID NO: 110, a CDRL2 of AAS, a CDRL3 of SEQ ID NO: 111, a CDRH1 of SEQ ID NO: 112, a CDRH2 of SEQ ID NO: 113, and a CDRH3 of SEQ ID NO: 114.

106. The fusion protein of any one of claims 1 to 80, and 100 to 105, wherein the PD-1 antibody comprises, comprising:
(i) a light chain variable region of SEQ ID NO: 34 and a heavy chain variable region of SEQ ID NO: 35;
(ii) a light chain variable region of SEQ ID NO: 43 and a heavy chain variable region of SEQ ID NO: 44;
(iii) a light chain variable region of SEQ ID NO: 52 and a heavy chain variable region of SEQ ID NO: 53;
(iv) a light chain variable region of SEQ ID NO: 61 and a heavy chain variable region of SEQ ID NO: 62;
(v) a light chain variable region of SEQ ID NO: 70 and a heavy chain variable region of SEQ ID NO: 71;
(vi) a light chain variable region of SEQ ID NO: 79 and a heavy chain variable region of SEQ ID NO: 80;
(vii) a light chain variable region of SEQ ID NO: 88 and a heavy chain variable region of SEQ ID NO: 89;
(viii) a light chain variable region of SEQ ID NO: 97 and a heavy chain variable region of SEQ ID NO: 98;
(ix) a light chain variable region of SEQ ID NO: 106 and a heavy chain variable region of SEQ ID NO: 107; or
(x) a light chain variable region of SEQ ID NO: 115 and a heavy chain variable region of SEQ ID NO: 116.

107. The fusion protein of any one of claims 1 to 80, and 100 to 106, wherein the PD-1 antibody comprises:
(i) a light chain of SEQ ID NO: 36 and a heavy chain of SEQ ID NO: 37;
(ii) a light chain of SEQ ID NO: 45 and a heavy chain of SEQ ID NO: 46;
(iii) a light chain of SEQ ID NO: 54 and a heavy chain of SEQ ID NO: 55;
(iv) a light chain of SEQ ID NO: 63 and a heavy chain of SEQ ID NO: 64;
(v) a light chain of SEQ ID NO: 72 and a heavy chain of SEQ ID NO: 73;
(vi) a light chain of SEQ ID NO: 81 and a heavy chain of SEQ ID NO: 82;
(vii) a light chain of SEQ ID NO: 90 and a heavy chain of SEQ ID NO: 91;
(viii) a light chain of SEQ ID NO: 99 and a heavy chain of SEQ ID NO: 100;
(ix) a light chain of SEQ ID NO: 108 and a heavy chain of SEQ ID NO: 109; or
(x) a light chain of SEQ ID NO: 117 and a heavy chain of SEQ ID NO: 118.

108. The fusion protein of any one of claims 1 to 80, and 100 to 107, wherein the anti-PD-1 antibody is a human or humanized antibody.

109. The fusion protein of any one of claims 1 to 80, and 100 to 108, wherein the anti-PD-1 antibody, wherein the intact anti-PD-1 antibody is an IgA, IgD, IgE, IgG, or IgM antibody.

110. The fusion protein of any one of claims 1 to 80, and 100 to 109, wherein the anti-PD-1 antibody is an IgG antibody.

111. The fusion protein of any one of claims 1 to 80, and 100 to 110, wherein the anti-PD-1 antibody is an IgG1 or IgG4 antibody.

112. The fusion protein of any one of claims 1 to 80, and 100 to 111, wherein the anti-PD-1 antibody is in a knobs-in-holes configuration.

113. The fusion protein of any one of claims 1 to 80, and 100 to 112, wherein the anti-PD-1 antibody is a non-blocking antibody.

114. The fusion protein of any one of claims 1 to 80, and 100 to 112, wherein the anti-PD-1 antibody is a blocking antibody.

115. The fusion protein of claim 1, where the fusion protein is:
fusion protein A1 comprising amino acid sequences of SEQ ID NOs: 36, 292, and 300;
fusion protein A2 comprising amino acid sequences of SEQ ID NOs: 36, 293, and 300;
fusion protein A3 comprising amino acid sequences of SEQ ID NOs: 36, 294, and 300;
fusion protein A4 comprising amino acid sequences of SEQ ID NOs: 36, 295, and 300;
fusion protein A5 comprising amino acid sequences of SEQ ID NOs: 36, 296, and 300;
fusion protein A6 comprising amino acid sequences of SEQ ID NOs: 36, 297, and 300; or
fusion protein A7 comprising amino acid sequences of SEQ ID NOs: 36, 298, and 300.

116. A pharmaceutical composition comprising the fusion protein of any one of claims 1 to 115 or the IL-2 mutein of any one of claims 2 to 80, and a pharmaceutically acceptable excipient.

117. The pharmaceutical composition of claim 116, wherein the pharmaceutical composition is in single dosage form.

118. The pharmaceutical composition of claim 116 or 117, wherein the pharmaceutical composition is a solid.

119. The pharmaceutical composition of claim 116 or 117, wherein the pharmaceutical composition is in a parenteral dosage form.

120. The pharmaceutical composition of claim 119, the pharmaceutical composition is in an intravenous dosage form.

121. The pharmaceutical composition of any one of claims 116, 117, 119, or 120, wherein the pharmaceutical composition is a solution.

122. A method of treating one or more symptoms of treating, preventing, or ameliorating one or more symptoms of a proliferative disease in a subject, comprising administering to the subject in need thereof a therapeutically effective amount of the fusion protein of any one of claims 1 to 115, the IL-2 mutein of any one of claims 2 to 80, or the pharmaceutical composition of any one of claims 116 to 121.

123. The method of claim 122, wherein the proliferative disease is cancer.

124. The method of claim 122 or 123, wherein the proliferative disease is metastatic cancer.

125. A method of inhibiting the growth of a cell, comprising contacting the cell with an effective amount of the fusion protein of any one of claims 1 to 115, the IL-2 mutein of any one of claims 2 to 80, or the pharmaceutical composition of any one of claims 116 to 121.

126. The method of claim 125, wherein the cell is a cancerous cell.


SEQUENCE LISTING (301 sequences)

SEQ ID NO: 1 - protein, 8 aa, synthetic construct
  REGION 1..8: note = Based on llima sequence
GSTWSINT

SEQ ID NO: 2 - protein, 7 aa, synthetic construct
  REGION 1..7: note = Based on llima sequence
ISSGGST

SEQ ID NO: 3 - protein, 10 aa, synthetic construct
  REGION 1..10: note = Based on llima sequence
YAQSTWYPPS

SEQ ID NO: 4 - protein, 25 aa, Homo sapiens [sequence omitted]

SEQ ID NO: 5 - protein, 17 aa, Homo sapiens
LAWYRQAPGKQRDLVAR

SEQ ID NO: 6 - protein, 38 aa, Homo sapiens
YYADSVKGRFTISRDNSKNTLYLQMNSLRAEDTAVYYC

SEQ ID NO: 7 - protein, 11 aa, Homo sapiens
WGQGTLVTVSS

SEQ ID NO: 8 - protein, 116 aa, synthetic construct
  REGION 1..116: note = Based on llima sequence
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSS

SEQ ID NO: 9 - protein, 8 aa, synthetic construct
  REGION 1..8: note = Based on llima sequence
GFAFRGFG

SEQ ID NO: 10 - protein, 8 aa, synthetic construct
  REGION 1..8: note = Based on llima sequence
INNGGSDT

SEQ ID NO: 11 - protein, 9 aa, synthetic construct
  REGION 1..9: note = Based on llima sequence
AIGGPGASP

SEQ ID NO: 12 - protein, 25 aa, Homo sapiens
QVQLVESGGGVVQPGGSLRLSCAAS

SEQ ID NO: 13 - protein, 17 aa, Homo sapiens
MSWVRQAPGKGLEWVSS

SEQ ID NO: 14 - protein, 11 aa, Homo sapiens
SGQGTQVTVSS

SEQ ID NO: 15 - protein, 116 aa, synthetic construct
  REGION 1..116: note = Based on llima sequence
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSS

SEQ ID NO: 16 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 28 - protein, 107 aa, Homo sapiens [sequence omitted]

SEQ ID NO: 29 - protein, 6 aa, Homo sapiens
QSVSSY

SEQ ID NO: 30 - protein, 9 aa, Homo sapiens
QQSSNWPRT

SEQ ID NO: 31 - protein, 8 aa, Homo sapiens
GITFSNSG

SEQ ID NO: 32 - protein, 8 aa, Homo sapiens
IWYDGSKR

SEQ ID NO: 33 - protein, 6 aa, Homo sapiens
ATNDDY

SEQ ID NO: 34 - protein, 107 aa, Homo sapiens [sequence omitted]

SEQ ID NO: 35 - protein, 113 aa, Homo sapiens [sequence omitted]

SEQ ID NO: 36 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 10 aa, Homo sapiens
KGVSTSGYSY

SEQ ID NO: 39 - protein, 9 aa, Homo sapiens
QHSRDLPLT

SEQ ID NO: 40 - protein, 8 aa, Homo sapiens
GYTFTNYY

SEQ ID NO: 41 - protein, 8 aa, Homo sapiens
INPSNGGT

SEQ ID NO: 42 - protein, 13 aa, Homo sapiens
ARRDYRFDMGFDY

SEQ ID NO: 43 - protein, 111 aa, Homo sapiens [sequence omitted]

SEQ ID NO: 44 - protein, 120 aa, Homo sapiens [sequence omitted]

SEQ ID NO: 45 - protein, 218 aa, Homo sapiens
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 447 aa, Homo sapiens
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 11 aa, Homo sapiens
QSIVHSNGNTY

SEQ ID NO: 48 - protein, 9 aa, Homo sapiens
FQGSHVPLT

SEQ ID NO: 49 - protein, 8 aa, Homo sapiens
GYTFTDYE

SEQ ID NO: 50 - protein, 8 aa, Homo sapiens
IESETGGT

SEQ ID NO: 51 - protein, 18 aa, Homo sapiens
AREGITTVATTYYWYFDV

SEQ ID NO: 52 - protein, 112 aa, Homo sapiens [sequence omitted]

SEQ ID NO: 53 - protein, 125 aa, Homo sapiens [sequence omitted]

SEQ ID NO: 54 - protein, 219 aa, Homo sapiens
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 452 aa, Homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 6 aa, Homo sapiens
QTIGTW

SEQ ID NO: 57 - protein, 9 aa, Homo sapiens
QQVYSIPWT

SEQ ID NO: 58 - protein, 8 aa, Homo sapiens
GFTFSSYM

SEQ ID NO: 59 - protein, 8 aa, Homo sapiens
ISGGGANT

SEQ ID NO: 60 - protein, 9 aa, Homo sapiens
ARQLYYFDY

SEQ ID NO: 61 - protein, 107 aa, Homo sapiens
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 62 - protein, 116 aa, Homo sapiens
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 63 - protein, 214 aa, Homo sapiens
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 443 aa, Homo sapiens
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 6 aa, Homo sapiens
QGISSW

SEQ ID NO: 66 - protein, 9 aa, Homo sapiens
QQANHLPFT

SEQ ID NO: 67 - protein, 8 aa, Homo sapiens
GGTFSSYA

SEQ ID NO: 68 - protein, 8 aa, Homo sapiens
IIPMFDTA

SEQ ID NO: 69 - protein, 13 aa, Homo sapiens
ARAEHSSTGTFDY

SEQ ID NO: 70 - protein, 107 aa, Homo sapiens
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

SEQ ID NO: 71 - protein, 120 aa, Homo sapiens
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 72 - protein, 214 aa, Homo sapiens
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 447 aa, Homo sapiens
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 12 aa, Homo sapiens
QSLLDSGNQKNF

SEQ ID NO: 75 - protein, 9 aa, Homo sapiens
QNDYSYPYT

SEQ ID NO: 76 - protein, 8 aa, Homo sapiens
GYTFTTYW

SEQ ID NO: 77 - protein, 8 aa, Homo sapiens
IYPGTGGS

SEQ ID NO: 78 - protein, 10 aa, Homo sapiens
TRWTTGTGAY

SEQ ID NO: 79 - protein, 113 aa, Homo sapiens
EIVLTQSPATLSLSPGERATLSCKSSQSLLDSGNQKNFLTWYQQKPGQAPRLLIYWASTRESGVPSRFSGSGSGTDFTETISSLEAEDAATYYCQNDYSYPYTFGQGTKVEIK

SEQ ID NO: 80 - protein, 117 aa, Homo sapiens
EVQLVQSGAEVKKPGESLRISCKGSGYTFTTYWMHWVRQATGQGLEWMGNIYPGTGGSNFDEKEKNRVTITADKSTSTAYMELSSLRSEDTAVYYCTRWTTGTGAYWGQGTTVTVSS

SEQ ID NO: 81 - protein, 220 aa, Homo sapiens
EIVLTQSPATLSLSPGERATLSCKSSQSLLDSGNQKNFLTWYQQKPGQAPRLLIYWASTRESGVPSRFSGSGSGTDFTETISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 444 aa, Homo sapiens
EVQLVQSGAEVKKPGESLRISCKGSGYTFTTYWMHWVRQATGQGLEWMGNIYPGTGGSNFDEKEKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLQG

SEQ ID NO: 83 - protein, 6 aa, Homo sapiens
ESVSND

SEQ ID NO: 84 - protein, 9 aa, Homo sapiens
HQAYSSPYT

SEQ ID NO: 85 - protein, 8 aa, Homo sapiens
GFSLTSYG

SEQ ID NO: 86 - protein, 7 aa, Homo sapiens
IYADGST

SEQ ID NO: 87 - protein, 12 aa, Homo sapiens
ARAYGNYWYIDV

SEQ ID NO: 88 - protein, 107 aa, Homo sapiens
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 89 - protein, 118 aa, Homo sapiens
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 90 - protein, 214 aa, Homo sapiens
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 446 aa, Homo sapiens
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQENWYVDGVEVHNAKTKPREEQENSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGIK

SEQ ID NO: 92 - protein, 6 aa, Homo sapiens
LSINTF

SEQ ID NO: 93 - protein, 9 aa, Homo sapiens
QQSSNTPFT

SEQ ID NO: 94 - protein, 8 aa, Homo sapiens
GFTFSNFG

SEQ ID NO: 95 - protein, 8 aa, Homo sapiens
ISGGGRDT

SEQ ID NO: 96 - protein, 10 aa, Homo sapiens
VKWGNIYFDY

SEQ ID NO: 97 - protein, 107 aa, Homo sapiens [sequence omitted]

SEQ ID NO: 98 - protein, 117 aa, Homo sapiens [sequence omitted]

SEQ ID NO: 99 - protein, 214 aa, Homo sapiens
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 444 aa, Homo sapiens
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 6 aa, Homo sapiens
QDVTTA

SEQ ID NO: 102 - protein, 9 aa, Homo sapiens
QQHYTIPWT

SEQ ID NO: 103 - protein, 8 aa, Homo sapiens
GFTFSNYG

SEQ ID NO: 104 - protein, 8 aa, Homo sapiens
ISGGGSNI

SEQ ID NO: 105 - protein, 9 aa, Homo sapiens
VSYYYGIDF

SEQ ID NO: 106 - protein, 107 aa, Homo sapiens
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIK

SEQ ID NO: 107 - protein, 116 aa, Homo sapiens
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSS

SEQ ID NO: 108 - protein, 214 aa, Homo sapiens
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 443 aa, Homo sapiens
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 10 aa, Homo sapiens
ESVDNYGMSF

SEQ ID NO: 111 - protein, 9 aa, Homo sapiens
QQSKEVPYT

SEQ ID NO: 112 - protein, 8 aa, Homo sapiens
GYSFTSYW

SEQ ID NO: 113 - protein, 8 aa, Homo sapiens
IHPSDSET

SEQ ID NO: 114 - protein, 12 aa, Homo sapiens
AREHYGTSPFAY

SEQ ID NO: 115 - protein, 111 aa, Homo sapiens
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 116 - protein, 119 aa, Homo sapiens
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 117 - protein, 218 aa, Homo sapiens
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 445 aa, Homo sapiens
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 119 - protein, 5 aa, Homo sapiens
GSRIA

SEQ ID NO: 120 - protein, 8 aa, Homo sapiens
ISQNGGST

SEQ ID NO: 121 - protein, 22 aa, Homo sapiens
AAALMTWYGTSYCPHLSTEFGF

SEQ ID NO: 122 - protein, 126 aa, Homo sapiens
QVKLEESGGGSVQAGGSLRLSCAASGSRIAMGWFRQAPGKEREFVGAISQNGGSTCYADSVKDRFTISRDNAKNMVYLQMNSLKPEDTAVYYCAAALMTWYGTSYCPHLSTEFGFWGQGTLVTVSS

SEQ ID NO: 123 - protein, 8 aa, Homo sapiens
GFTLDDYA

SEQ ID NO: 124 - protein, 8 aa, Homo sapiens
ISSSDGST

SEQ ID NO: 125 - protein, 15 aa, Homo sapiens
STDVGCYGSTSPEAY

SEQ ID NO: 126 - protein, 122 aa, Homo sapiens
QVKLEESGGGLVQPGGSLRLSCAASGFTLDDYALGWFRQAPGKEREGVSCISSSDGSTYYADSVKGRFTISRDNAKNTVYLQMNSLKPEDTAVYYCSTDVGCYGSTSPEAYWGKGTLVTVSS

SEQ ID NO: 127 - protein, 8 aa, Homo sapiens
GRTFSSYD

SEQ ID NO: 128 - protein, 8 aa, Homo sapiens
VSASGGRS

SEQ ID NO: 129 - protein, 18 aa, Homo sapiens
AARNGAYYITIDPAQYNY

SEQ ID NO: 130 - protein, 125 aa, Homo sapiens
QVQLVDSGGGLVQAGGSLRLSCAASGRTFSSYDFGWFRQAPGKEREFVAAVSASGGRSGYSDFAKDRFTTSRENAKNTVYLQMNSLKPEDTAVYYCAARNGAYYITIDPAQYNYWGQGTLVTVSS

SEQ ID NO: 131 - protein, 8 aa, Homo sapiens
GRTFSSYA

SEQ ID NO: 132 - protein, 8 aa, Homo sapiens
ISWSGGIT

SEQ ID NO: 133 - protein, 13 aa, Homo sapiens
AADLNQESSWYDY

SEQ ID NO: 134 - protein, 120 aa, Homo sapiens
QVQLVESGGGLVQAGDSLTLSCAASGRTFSSYAMGWFRQAPGKEREFVAAISWSGGITYYEDSVKGRFTISRDNAKNTVYLQMNSLKPEDTAVYYCAADLNQESSWYDYWGQGTQVTVSS

SEQ ID NO: 135 - protein, 5 aa, Homo sapiens
GSRYA

SEQ ID NO: 136 - protein, 8 aa, Homo sapiens
ISWNGGST

SEQ ID NO: 137 - protein, 22 aa, Homo sapiens
AAALIPKYGTSYCPHISTEFDF

SEQ ID NO: 138 - protein, 126 aa, Homo sapiens
QVKLEESGGGSVQAGGSLRLSCAGSGSRYAMGWFRQAPGKEREFVAAISWNGGSTCYADSVKGRFTISRDNAKNMVYLQMNSLKPEDTAVYYCAAALIPKYGTSYCPHISTEFDFWGQGTQVTVSS

SEQ ID NO: 139 - protein, 6 aa, synthetic construct
  REGION 1..6: note = Linker
GSGGSG

SEQ ID NO: 140 - protein, 9 aa, synthetic construct
  REGION 1..9: note = Linker
GSGGSGGSG

SEQ ID NO: 141 - protein, 12 aa, synthetic construct
  REGION 1..12: note = Linker
GSGGSGGSGGSG

SEQ ID NO: 142 - protein, 4 aa, synthetic construct
  REGION 1..4: note = Linker
GGGS

SEQ ID NO: 143 - protein, 8 aa, synthetic construct
  REGION 1..8: note = Linker
GGGSGGGS

SEQ ID NO: 144 - protein, 12 aa, synthetic construct
  REGION 1..12: note = Linker
GGGSGGGSGGGS

SEQ ID NO: 145 - protein, 16 aa, synthetic construct
  REGION 1..16: note = Linker
GGGSGGGSGGGSGGGS

SEQ ID NO: 146 - protein, 5 aa, synthetic construct
  REGION 1..5: note = Linker
GGGGS

SEQ ID NO: 147 - protein, 10 aa, synthetic construct
  REGION 1..10: note = Linker
GGGGSGGGGS

SEQ ID NO: 148 - protein, 15 aa, synthetic construct
  REGION 1..15: note = Linker
GGGGSGGGGSGGGGS

SEQ ID NO: 149 - protein, 20 aa, synthetic construct
  REGION 1..20: note = Linker
GGGGSGGGGSGGGGSGGGGS

SEQ ID NO: 150 - protein, 4 aa, synthetic construct
  REGION 1..4: note = Linker
SGSG

SEQ ID NO: 151 - protein, 7 aa, synthetic construct
  REGION 1..7: note = Linker
SGSGGSG

SEQ ID NO: 152 - protein, 10 aa, synthetic construct
  REGION 1..10: note = Linker
SGSGGSGGSG

SEQ ID NO: 153 - protein, 13 aa, synthetic construct
  REGION 1..13: note = Linker
SGSGGSGGSGGSG

SEQ ID NO: 154 - protein, 5 aa, synthetic construct
  REGION 1..5: note = Linker
SGGGS

SEQ ID NO: 155 - protein, 9 aa, synthetic construct
  REGION 1..9: note = Linker
SGGGSGGGS

SEQ ID NO: 156 - protein, 13 aa, synthetic construct
  REGION 1..13: note = Linker
SGGGSGGGSGGGS

SEQ ID NO: 157 - protein, 17 aa, synthetic construct
  REGION 1..17: note = Linker
SGGGSGGGSGGGSGGGS

SEQ ID NO: 158 - protein, 6 aa, synthetic construct
  REGION 1..6: note = Linker
SGGGGS

SEQ ID NO: 159 - protein, 11 aa, synthetic construct
  REGION 1..11: note = Linker
SGGGGSGGGGS

SEQ ID NO: 160 - protein, 16 aa, synthetic construct
  REGION 1..16: note = Linker
SGGGGSGGGGSGGGGS

SEQ ID NO: 161 - protein, 21 aa, synthetic construct
  REGION 1..21: note = Linker
SGGGGSGGGGSGGGGSGGGGS

SEQ ID NO: 162 - protein, 5 aa, synthetic construct
  REGION 1..5: note = Linker
EAAAK

SEQ ID NO: 163 - protein, 10 aa, synthetic construct
  REGION 1..10: note = Linker
EAAAKEAAAK

SEQ ID NO: 164 - protein, 15 aa, synthetic construct
  REGION 1..15: note = Linker
EAAAKEAAAKEAAAK

SEQ ID NO: 165 - protein, 20 aa, synthetic construct
  REGION 1..20: note = Linker
EAAAKEAAAKEAAAKEAAAK

SEQ ID NO: 166 - protein, 5 aa, synthetic construct
  REGION 1..5: note = Linker
PAPAP

SEQ ID NO: 167 - protein, 10 aa, synthetic construct
  REGION 1..10: note = Linker
PAPAPPAPAP

SEQ ID NO: 168 - protein, 15 aa, synthetic construct
  REGION 1..15: note = Linker
PAPAPPAPAPPAPAP

SEQ ID NO: 169 - protein, 20 aa, synthetic construct
  REGION 1..20: note = Linker
PAPAPPAPAPPAPAPPAPAP

SEQ ID NO: 170 - protein, 8 aa, synthetic construct
  REGION 1..8: note = Linker
IKRTVAAP

SEQ ID NO: 171 - protein, 5 aa, synthetic construct
  REGION 1..5: note = Linker
RAKPS

SEQ ID NO: 172 - protein, 10 aa, synthetic construct
  REGION 1..10: note = Linker
RAKPSRAKPS

SEQ ID NO: 173 - protein, 5 aa, synthetic construct
  REGION 1..5: note = Linker
ASTKG

SEQ ID NO: 174 - protein, 10 aa, synthetic construct
  REGION 1..10: note = Linker
ASTKGASTKG

SEQ ID NO: 175 - protein, 5 aa, synthetic construct
  REGION 1..5: note = Linker
AKTHT

SEQ ID NO: 176 - protein, 10 aa, synthetic construct
  REGION 1..10: note = Linker
AKTHTAKTHT

SEQ ID NO: 177 - protein, 15 aa, synthetic construct
  REGION 1..15: note = Linker
ASTKGGGGGSGGGGS

SEQ ID NO: 178 - protein, 15 aa, synthetic construct
  REGION 1..15: note = Linker
RAKPSGGGGSGGGGS

SEQ ID NO: 179 - protein, 133 aa, Homo sapiens [sequence omitted]

SEQ ID NO: 180 - protein, 133 aa, Homo sapiens
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 181 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 182 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 183 - protein, 133 aa, Homo sapiens
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 184 - protein, 133 aa, Homo sapiens
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 185 - protein, 133 aa, Homo sapiens
APTSSSTKKTQLQLEHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 186 - protein, 133 aa, Homo sapiens
APASSSTKKTQLQLEHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 187 - protein, 133 aa, Homo sapiens
APTSSSTKKTQLQLKHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 188 - protein, 133 aa, Homo sapiens
APASSSTKKTQLQLKHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 189 - protein, 133 aa, Homo sapiens
APTSSSTKKTQLQLKHLLLDLQMILNGINNYENPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 190 - protein, 133 aa, Homo sapiens
APASSSTKKTQLQLKHLLLDLQMILNGINNYENPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 191 - protein, 133 aa, Homo sapiens
APTSSSTKKTQLQLKHLLLDLQMILNGINNYKNPKLTNMTTFKFYMPKKATELKHLQCLEEELKPLEEVLNLAQSENFHLRPRDLISNINVIVLELKGSETTFMCEYADETATIVEFLNRWITFSQSIISTLT

SEQ ID NO: 192 - protein, 133 aa, Homo sapiens
APASSSTKKTQLQLKHLLLDLQMILNGINNYKNPKLTNMTTFKFYMPKKATELKHLQCLEEELKPLEEVLNLAQSENFHLRPRDLISNINVIVLELKGSETTFMCEYADETATIVEFLNRWITFSQSIISTLT

SEQ ID NO: 193 - protein, 133 aa, Homo sapiens
APTSSSTKKTQLQLEHLLLDLQMILNGINNYENPKLTEMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 194 - protein, 133 aa, Homo sapiens
APASSSTKKTQLQLEHLLLDLQMILNGINNYENPKLTEMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 195 - protein, 133 aa, Homo sapiens
APTSSSTKKTQLQLEHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 196 - protein, 133 aa, Homo sapiens
APASSSTKKTQLQLEHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 197 - protein, 133 aa, Homo sapiens
APTSSSTKKTQLQLKHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 198 - protein, 133 aa, Homo sapiens
APASSSTKKTQLQLKHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 199 - protein, 133 aa, Homo sapiens
APTSSSTKKTQLQLEHLLLDLQMILNGINNYKNPKLTEMLTAKFYMPKKATELKHLQCLEEELKPLEEVLNLAQSENFHLRPRDLISNINVIVLELKGSETTFMCEYADETATIVEFLNRWITFSQSIISTLT

SEQ ID NO: 200 - protein, 133 aa, Homo sapiens
APASSSTKKTQLQLEHLLLDLQMILNGINNYKNPKLTEMLTAKFYMPKKATELKHLQCLEEELKPLEEVLNLAQSENFHLRPRDLISNINVIVLELKGSETTFMCEYADETATIVEFLNRWITFSQSIISTLT

SEQ ID NO: 201 - protein, 133 aa, Homo sapiens
APTSSSTKKTQLQLEHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 202 - protein, 133 aa, Homo sapiens
APASSSTKKTQLQLEHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 203 - protein, 133 aa, Homo sapiens
APTSSSTKKTQLQLKHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 204 - protein, 133 aa, Homo sapiens
APASSSTKKTQLQLKHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 205 - protein, 133 aa, Homo sapiens
APTSSSTKKTQLQLEHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 206 - protein, 133 aa, Homo sapiens
APASSSTKKTQLQLEHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 207 - protein, 133 aa, Homo sapiens
APTSSSTKKTQLQLEHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 208 - protein, 133 aa, Homo sapiens
APASSSTKKTQLQLEHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 209 - protein, 133 aa, Homo sapiens
APTSSSTKKTQLQLEHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 210 - protein, 133 aa, Homo sapiens
APASSSTKKTQLQLEHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 211 - protein, 133 aa, Homo sapiens
APTSSSTKKTQLQLKHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 212 - protein, 133 aa, Homo sapiens
APASSSTKKTQLQLKHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 213 - protein, 133 aa, Homo sapiens
APTSSSTKKTQLQLKHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 214 - protein, 133 aa, Homo sapiens
APASSSTKKTQLQLKHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 215 - protein, 133 aa, Homo sapiens
APTSSSTKKTQLQLKHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 216 - protein, 133 aa, Homo sapiens
APASSSTKKTQLQLKHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 217 - protein, 12 aa, Homo sapiens
NNYKNPKLTRML

SEQ ID NO: 218 - protein, 8 aa, Homo sapiens
QSMEIDAT

SEQ ID NO: 219 - protein, 8 aa, Homo sapiens
QSMHIDAT

SEQ ID NO: 220 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 221 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 222 - protein, 129 aa, Homo sapiens
APTSSSTKKTQLQLEHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 223 - protein, 129 aa, Homo sapiens
APASSSTKKTQLQLEHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 224 - protein, 129 aa, Homo sapiens
APTSSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 225 - protein, 129 aa, Homo sapiens
APASSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 226 - protein, 129 aa, Homo sapiens
APTSSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 227 - protein, 129 aa, Homo sapiens
APASSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 228 - protein, 129 aa, Homo sapiens
APTSSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 229 - protein, 129 aa, Homo sapiens
APASSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 230 - protein, 129 aa, Homo sapiens
APTSSSTKKTQLQLEELLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 231 - protein, 129 aa, Homo sapiens
APASSSTKKTQLQLEELLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 232 - protein, 129 aa, Homo sapiens
APTSSSTKKTQLQLEFLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 233 - protein, 129 aa, Homo sapiens
APASSSTKKTQLQLEFLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 234 - protein, 129 aa, Homo sapiens
APTSSSTKKTQLQLEILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 235 - protein, 129 aa, Homo sapiens
APASSSTKKTQLQLEILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 236 - protein, 129 aa, Homo sapiens
APTSSSTKKTQLQLEVLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 237 - protein, 129 aa, Homo sapiens
APASSSTKKTQLQLEVLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 238 - protein, 129 aa, Homo sapiens
APTSSSTKKTQLQLEHLLLTLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 239 - protein, 129 aa, Homo sapiens
APASSSTKKTQLQLEHLLLTLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 240 - protein, 129 aa, Homo sapiens
APTSSSTKKTQLQLEHLLLALQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 241 - protein, 129 aa, Homo sapiens
APASSSTKKTQLQLEHLLLALQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 242 - protein, 129 aa, Homo sapiens
APTSSSTKKTQLQLEHLLLELQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 243 - protein, 129 aa, Homo sapiens
APASSSTKKTQLQLEHLLLELQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 244 - protein, 129 aa, Homo sapiens
APTSSSTKKTQLQLEHLLLKLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 245 - protein, 129 aa, Homo sapiens
APASSSTKKTQLQLEHLLLKLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 246 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 247 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 248 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 249 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 250 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVGVLELKGSETTFMCEYADETATIVEFLNRWITFSQSIISTLT

SEQ ID NO: 251 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVGVLELKGSETTFMCEYADETATIVEFLNRWITFSQSIISTLT

SEQ ID NO: 252 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVDVLELKGSETTFMCEYADETATIVEFLNRWITFSQSIISTLT

SEQ ID NO: 253 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVDVLELKGSETTFMCEYADETATIVEFLNRWITFSQSIISTLT

SEQ ID NO: 254 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVEVLELKGSETTFMCEYADETATIVEFLNRWITFSQSIISTLT

SEQ ID NO: 255 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVEVLELKGSETTFMCEYADETATIVEFLNRWITFSQSIISTLT

SEQ ID NO: 256 - protein, 129 aa, Homo sapiens
APTSSSTKKTQLQLEELLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 257 - protein, 129 aa, Homo sapiens
APASSSTKKTQLQLEELLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 258 - protein, 129 aa, Homo sapiens
APTSSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 259 - protein, 129 aa, Homo sapiens
APASSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 260 - protein, 129 aa, Homo sapiens
APTSSSTKKTQLQLKILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 261 - protein, 129 aa, Homo sapiens
APTSSSTKKTQLQLKHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 262 - protein, 129 aa, Homo sapiens
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 263 - protein, 129 aa, Homo sapiens
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 264 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 265 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 266 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSKSIISTLT

SEQ ID NO: 267 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSKSIISTLT

SEQ ID NO: 268 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSRSIISTLT

SEQ ID NO: 269 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSRSIISTLT

SEQ ID NO: 270 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSSSIISTLT

SEQ ID NO: 271 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSSSIISTLT

SEQ ID NO: 272 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIISTLT

SEQ ID NO: 273 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIISTLT

SEQ ID NO: 274 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIIRTLT

SEQ ID NO: 275 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIIRTLT

SEQ ID NO: 276 - protein, 129 aa, Homo sapiens
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 277 - protein, 129 aa, Homo sapiens
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 278 - protein, 129 aa, Homo sapiens
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 279 - protein, 129 aa, Homo sapiens
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 280 - protein, 129 aa, Homo sapiens
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCRSIISTLT

SEQ ID NO: 281 - protein, 129 aa, Homo sapiens
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCRSIISTLT

SEQ ID NO: 282 - protein, 129 aa, Homo sapiens
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 283 - protein, 129 aa, Homo sapiens
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 284 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSESIISTLT

SEQ ID NO: 285 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSESIISTLT

SEQ ID NO: 286 - protein, 129 aa, Homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIIRTLT

SEQ ID NO: 287 - protein, 129 aa, Homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIIRTLT

SEQ ID NO: 288 - protein, 145 aa, Homo sapiens
APTSSSTSSSTAEAQQQQQQQQQQQQHLEQLLMDLQELLSRMQSMEIDATTFKFYLPKQATELKDLQCLEDELGPLRHVLDLTQSKSFQLEDAENFISNIRVTVVKLKGSDNTFECQFDDESATVVDFLRRWIAFSQSIISTSPQ

SEQ ID NO: 289 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 290 - protein, 213 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 291 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 292 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 293 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 294 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIISTLT

SEQ ID NO: 295 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIIRTLT

SEQ ID NO: 296 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 297 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 298 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIIRTLT

SEQ ID NO: 299 - protein, 588 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 300 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 301 - protein, 129 aa, Homo sapiens
APASSSTKKTQLQLEHLSLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSESIISTLT